(19) United States
(12) Patent Application Publication
(10) Pub. No.: US 2025/0376535 A1
(43) Pub. Date: Dec. 11, 2025

(54) MONOSPECIFIC AND MULTI-SPECIFIC ANTIBODIES

(71) Applicant: BEIJING STARMAB BIOMED TECHNOLOGY LTD, Beijing (CN)

(72) Inventor: Yanbin Liang, Irvine, CA (US)

(21) Appl. No.: 18/878,518

(22) PCT Filed: Jun. 28, 2023
(86) PCT No.: PCT/US2023/069262

§ 371 Date: Dec. 23, 2024

Related U.S. Application Data
(60) Provisional application No. 63/356,385, filed on Jun. 28, 2022.
(60) Provisional application No. 63/434,814, filed on Dec. 22, 2022.

(51) Int. Cl. C07K 16/28; A61P 35/00; C07K 16/18; C07K 16/24

(52) U.S. Cl. CPC C07K 16/2878; A61P 35/00; C07K 16/18; C07K 16/244; C07K 16/2863; C07K 2317/31; C07K 2317/569; C07K 2317/622; C07K 2317/92

(57) ABSTRACT

Disclosed herein are monospecific and multi-specific antibodies with specificities for one or more of OX40, CD40, 4-1BB, HSA, IL-22, or EGFR. Specifically, the disclosure provides variable heavy (VHH) domains of the antibodies with specificities for one or more of OX40, CD40, 4-1BB, HSA, IL-22, or EGFR, and associated sequences. Further discloses are methods of using the monospecific or multi-specific antibodies for treating ah autoimmune disease.

### CROSS-REFERENCE TO RELATED APPLICATIONS

The present application claims the benefit of U.S. Provisional patent applications 63/356,385 filed Jun. 28, 2022 and 63/434,814 filed Dec. 22, 2022, the entire contents of both of which are incorporated by reference herein.

### SEQUENCE LISTING

A Sequence Listing is submitted herewith and incorporated by reference herein as an XML file created on Jun. 27, 2023, entitled “1959708-00014_Sequence_Listing.xml” and having a size of 138 KB.

### SUMMARY

Disclosed herein are monospecific VHH antibodies having specificity for OX-40, CD40, 4-1BB, HSA, IL-22 and epidermal growth factor receptor (EFGR), and multivalent single chain antibodies, incorporating two or more VHH domains having specificity for one or more of these antigens.

Some embodiments are single domain antibodies comprising, exclusively or primarily, a VHH domain of a camelid antibody. These embodiments are monospecific and monovalent.

Some embodiments comprise a VHH domain fused to one or more constant domains from a conventional antibody, for example the Fc region of a human IgG antibody. These embodiments are monospecific, but typically bivalent. Other valencies are possible depending, for example, on the choice of constant domains. The Fc regions of IgA and IgM can confer higher valency.

Some embodiments comprise two VHH domains with specificity for the same antigen joined in a single amino acid chain (a multivalent single chain antibody). These embodiments are also monospecific and bivalent. Additional VHH domains can be joined for higher valency.

Some embodiments comprise two (or more) VHH domains, wherein each VHH domain has specificity for a distinct antigen joined in a single amino acid chain (a multivalent, multi-specific single chain antibody). These embodiments are multivalent and multi-specific. In further embodiments comprising three or more VHH domains, two or more VHH domains may have specificity for a same antigen while one or more other VHH domains has specificity for a distinct antigen. Such constructs have a higher order valency than specificity,

Each of the monospecific embodiments have specificity for OX-40, CD40, HSA, IL-22, 4-1BB, or EFGR. Each of the multi-specific embodiments have specificity for one or more of OX-40, CD40, 4-1BB, HSA, IL-22, and EFGR, but may also have specificity for one or more other antigens.

In some embodiments comprising multiple antigen-binding domains an antigen-binding domain derived from a conventional VL-VH pairing can be used in place of one or more (but not all) of the VHH domains in the above embodiments.

The herein disclosed antigen-binding domains with specificity for a particular antigen may be referred to as means for binding the antigen.

### BRIEF DESCRIPTION OF THE DRAWINGS

FIG. 1 depicts a blocking assay of CD40 binding to CD40L by anti-CD40 antibody, pgDD40-HG-24.

FIG. 2 depicts blockage of CD40 binding to CED40L by an anti-CD40 VHH.

FIG. 3A-B depicts an ELISA binding analysis of SM2248. FIG. 3A depicts SM2248 binding to recombinant human CD47-Fc. FIG. 3B depicts SM2248 binding to recombinant human CD40-Fc.

FIG. 4A-D depicts flow cytometry binding analysis of SM2248 binding to cancer cell lines CHO-hCD47 (FIG. 4A), CHO-hCD40 (FIG. 4B), Raji (FIG. 4C), and A431 (FIG. 4D).

FIG. 5A-B depicts a cell-based potency assay. FIG. 5A depicts a HEK-NFKB reporter assay. FIG. 5B depicts SM2248 blocking SIRP binding to Jurkat cells.

FIG. 6 depicts 4-1BB59 blocking 4-1BB binding to 4-1BB-L.

FIG. 7A-B depicts ELISA binding analysis of SM2235-113. FIG. 7A depicts SM2235-113 binding to recombinant human EGFR-Fc. FIG. 7B depicts SM2235-1113 binding to recombinant CD16A.

FIG. 8A-D depicts flow cytometry binding analysis of SM2235 binding to cancer cell lines CHO-K1-EGFR (FIG. 8A), A431 (FIG. 8B), MB231 (FIG. 8C), and HCT116 (FIG. 8D).

FIG. 9A-B depicts flow cytometry binding analysis of SM2235 binding to CD16A on Jurkat-CD16A cells (FIG. 9A) and human NK cells (FIG. 9B).

FIG. 10 depicts blocking of EGF binding to EGFR on the CHO-EGFR overexpression cell line.

FIG. 11 depicts a human NK cell cytotoxicity assay, 10:1 NK: A431.

### DETAILED DESCRIPTION

Disclosed herein are monospecific immunoglobulin variable domains (referred to as VHH single domain antibodies), having specificity for OX-40, CD40, 4-1BB, HSA, IL-22 and epidermal growth factor receptor (EFGR), and multivalent single chain antibodies (MVSCA), incorporating the variable domains of two or more VHH, having specificity for one or more of these antigens.

As used herein, the term VHH refers to the variable domain of heavy-chain antibodies and is the antigen binding fragment of heavy chain only antibodies.

In some embodiments the MVSCA comprise two or more variable domains with specificity for the same antigen. That is, the MVSCA are multivalent, but monospecific with respect to antigen. In some of these embodiments the MVSCA comprises two or more iterations of a same VHH variable domain or multiple VHH variable domains each with specificity for the same epitope. That is, they are multivalent, but monospecific with respect to epitope. Such MVSCA will bind to only a single site on an antigen monomer, but can cross-link multiple copies of the monomer. In other of these embodiments the MVSCA comprises two or more VHH variable domains each with specificity for different epitopes of the same antigen. That is, they are multivalent, but multi-specific with respect to epitope. Such MVSCA may bind to multiple sites on an antigen monomer or cross-link multiple copies of the monomer.

In some embodiments the MVSCA comprise two or more VHH variable domains with specificity for distinct antigens, that is, they are multivalent and multi-specific with respect to antigen. In further embodiments, the MVSCA comprise multiple VHH variable domains wherein an additional variable domain is identical to a first VHH variable domain, wherein an additional VHH variable domain is different that a first VHH variable domain but is specific for a different epitope on a same antigen, or wherein an additional VHH variable domain is different that a first VHH variable domain but is specific for a different antigen, in any combination.

The MVSCA comprising two or more VHH variable domains may further comprise an immunoglobulin constant domain. For example, the C-terminal VHH variable domain can retain attachment to its original VHH constant domain. Alternatively, the C-terminal VHH variable domain can be attached to a constant domain or Fc region of a more conventional antibody, for example a human antibody, such as a human IgG antibody. In some embodiments a constant domain or complete Fc region may confer a particular functionality, as will be familiar to one of skill in the art. In other embodiments, the MVSCA comprising two or more VHH variable domains may further comprise a constant domain, wherein a constant domain is positioned between or N-terminally to the VHH variable domains instead of, or in addition to, being positioned C-terminally to the VHH variable domains.

### Antigens

OX40 (CD134; TNFRSF4) is a T cell costimulatory molecule of the tumor necrosis factor (TNF) receptor superfamily that coordinates with other co-stimulators (CD28, CD40, CD30, CD27, and 4-1BB) to manage the activation of the immune response. OX40 is upregulated on antigen activated CD4⁺ and CD8⁺ T cells with co-stimulation by CD40-CD40 ligand and CD28-B7. OX40 interactions with OX40 ligand on antigen-presenting cells enhances T cell survival, proliferation, and cytokine production. It also inhibits the conversion of effector T cells into regulatory T cells (Tregs) and can promote the maintenance of, and recall, response in memory T cells. OX40 is constitutively expressed on Tregs where it promotes Treg proliferation and immunosuppressive activity. OX40-OX40 ligand signaling is involved in allergic airway inflammation, graft-versus-host disease, and autoimmune disease.

CD40, also known as TNFRSF5, is a 45-50 kDa type I transmembrane glycoprotein member of the TNF receptor superfamily. Mature human CD40 consists of a 173 amino acid (aa) extracellular domain, a transmembrane domain, and a 62 aa cytoplasmic domain. The extracellular domain of human CD40 shares 58% and 56% aa sequence identity with mouse and rat CD40, respectively. An antagonistic soluble human CD40 splice variant contains an alternate sequence within the extracellular and transmembrane domains and lacks a cytoplasmic domain. CD40 is expressed on the surface of B cells, dendritic cells, macrophages, monocytes and platelets, as well as endothelial and epithelial cells. Interaction of CD40 with its ligand, CD40 ligand, leads to the aggregation of CD40 molecules resulting in the initiation of bidirectional intracellular signaling in both CD40 and CD40 ligand expressing cells. CD40 ligation by CD40 ligand promotes B cell activation and T cell-dependent humoral responses. CD40 serves multiple functions in both hematopoietic and epithelial cancers and is a target for tumor immunotherapy.

The epidermal growth factor receptor (EGFR) is a transmembrane protein that is a receptor for members of the epidermal growth factor (EGF) family of extracellular protein ligands. The EGFR is a member of the ErbB family of receptors, a subfamily of four closely related receptor tyrosine kinases: EGFR (ErbB-1), HER2/neu (ErbB-2), Her 3 (ErbB-3) and Her 4 (ErbB-4). In many cancer types, mutations affecting EGFR expression or activity could result in cancer. Epidermal growth factor receptor is a transmembrane protein that is activated by binding of its specific ligands, including epidermal growth factor and transforming growth factor α (TGFα). Deficient signaling of the EGFR and other receptor tyrosine kinases in humans is associated with diseases such as tumors, while over-expression is associated with the development of a wide variety of tumors. Interruption of EGFR signaling, either by blocking EGFR binding sites on the extracellular domain of the receptor or by inhibiting intracellular tyrosine kinase activity, can prevent the growth of EGFR-expressing tumors and improve the patient's condition.

4-1BB, also known as CD137 and TNFRSF9, is an approximately 30 kDa transmembrane glycoprotein in the TNF receptor superfamily. 4-1BB functions in the development and activation of multiple immune cells. Mature human 4-1BB consists of a 163 aa extracellular domain (ECD) with four TNFR cysteine-rich repeats (SEQ ID NO:41), a 27 aa transmembrane segment, and a 42 aa cytoplasmic domain. Within the ECD, human 4-1BB shares 60% aa sequence identity with mouse and rat 4-1BB. 4-1BB is expressed as a disulfide-linked homodimer on various populations of activated T cell including CD4+, CD8+,memory CD8+, NKT, and regulatory T cells as well as on myeloid and mast cell progenitors, dendritic cells, mast cells, and bacterially infected osteoblasts. It binds with high affinity to the transmembrane 4-1BB ligand/TNFSF9 which is expressed on antigen presenting cells and myeloid progenitor cells. This interaction co-stimulates the proliferation, activation, and/or survival of the 4-1BB expressing cell. It can also enhance the activation-induced cell death of repetitively stimulated T cells. Mice lacking 4-1BB show augmented T cell activation, perhaps due to its absence on regulatory T cells. 4-1BB can associate with OX40 on activated T cells, forming a complex that responds to either ligand and inhibits Treg and CD8+ T cell proliferation. Reverse signaling through 4-1BB ligand inhibits the development of dendritic cells, B cells, and osteoclasts but supports mature dendritic cell survival and co-stimulates the proliferation and activation of mast cells. 4-1BB activation enhances CD8+ T cell and NK cell mediated anti-tumor immunity. It also contributes to the development of inflammation in high fat diet-induced metabolic syndrome. Soluble forms of 4-1BB and 4-1BB ligand circulate at elevated levels in the serum of rheumatoid arthritis and hematologic cancer patients, respectively.

Human serum albumin (HSA) is the serum albumin found in human blood. It is the most abundant protein in human blood plasma; it constitutes about half of serum protein. It is produced in the liver. It is soluble in water and monomeric. Albumin transports hormones, fatty acids, and other compounds, buffers pH, and maintains oncotic pressure, among other functions. Albumin is synthesized in the liver as preproalbumin, which has an N-terminal peptide that is removed before the nascent protein is released from the rough endoplasmic reticulum. The product, proalbumin, is in turn cleaved in the Golgi vesicles to produce the secreted albumin. The reference range for albumin concentrations in serum is approximately 35-50 g/L (3.5-5.0 g/dL). It has a serum half-life of approximately 20 days. It has a molecular mass of 66.5 kDa. The long serum half-life of albumin is achieved in part by its size, 66 kDa, which prevents clearance through the kidney, and by its interaction with the neonatal Fc receptor (FcRn). Fusion to the anti-albumin VHH has been used to increase the half-life of the antitumor nanobody from 1-2 h to approximate 10 days.

Interleukin-22 (IL-22), also known as IL-10-related T cell-derived inducible factor (IL-TIF), was initially identified as a gene induced by IL-9 in mouse T cells and mast cells. Human IL-22 cDNA encodes a 179 amino acid (aa) residue protein with a putative 33 aa signal peptide that is cleaved to generate a 147 aa mature protein that shares approximately 79% and 22% aa sequence identity with mouse IL-22 and human IL-10, respectively. The human IL-22 gene is localized to chromosome 12q15. Although it exists as a single copy gene in human and in many mouse strains, the mouse IL-22 gene is duplicated in some mouse strains including C57B1/6, FVB and 129. The two mouse genes designated IL-TIF alpha and IL-TIF beta, share greater than 98% sequence homology in their coding region. IL-22 has been shown to activate STAT-1 and STAT-3 in several hepatoma cell lines and upregulate the production of acute phase proteins. IL-22 is produced by normal T cells upon anti-CD3 stimulation in humans. Mouse IL-22 expression is also induced in various organs upon lipopolysaccharide injection, suggesting that IL-22 may be involved in inflammatory responses. The functional IL-22 receptor complex consists of two receptor subunits, IL-22R (previously an orphan receptor named CRF2-9) and IL-10R beta (previously known as CRF2-4), belonging to the class II cytokine receptor family.

### Antibodies

Antibodies, and their use for treatment of diseases, are well known in the art. As used herein, the term “antibody” refers to a monomeric or multimeric protein comprising one or more polypeptide chains that comprise antigen-binding sites. An antibody binds specifically to an antigen and may be able to modulate the biological activity of the antigen. As used herein, the term “antibody” can include “full length antibody” and “antibody fragments.” The terms “binding site” or “antigen-binding site” as used herein denotes the region(s) of an antibody molecule to which a ligand actually binds. The term “antigen-binding site” comprises an antibody heavy chain variable domain (VH) and an antibody light chain variable domain (VL), or in the case of heavy chain only antibodies, an antibody heavy chain variable region.

Antibody specificity refers to selective recognition of the antibody for a particular epitope of an antigen. Natural antibodies, for example, are monospecific. The term “monospecific” antibody as used herein denotes an antibody that has one or more binding sites each of which bind to the same epitope of the same antigen. The monospecific antibodies disclosed herein are specific for OX-40, CD40, 4-1BB, HSA, IL-22, or EFGR. In some embodiments, monospecific antibodies comprise only the VHH domain heavy chain. In other embodiments, the monospecific antibody comprises a VHH domain fused to one or more protein domains including, for example, a human Fc region. In still other embodiments, the monospecific antibodies comprise a VHH as the only complete protein domain, that is, a single domain antibody. In some embodiments, the single domain antibody may additionally comprise a short peptide, such as a His-tag. A VHH domain may be referred to as means for binding a particular target (such as, OX-40, CD40, 4-1BB, HSA, IL-22, or EFGR). Any of the various antibody structures, formats, or constructs disclosed herein that contains a VHH domain or is constructed to contain a VHH domain can thus be referred to an antibody comprising means for binding the indicated target. Some embodiments may specifically include one or more particular antibody structures, formats, or constructs. Other embodiments may specifically exclude one or more particular antibody structures, formats, or constructs.

As used herein “an antibody having specificity for”, “an antibody recognizing”, “an antibody having affinity for”, “an antibody with a binding site for”, and similar constructions may be used interchangeably.

“Multi-specific antibodies” refers to antibodies that have two or more antigen-binding specificities. Multi-specific antibodies disclosed herein are specific for at least two of OX-40, CD40, 4-1BB, HSA, IL-22, and EFGR, or for at least one of the foregoing specificities and at least a second specificity. In some embodiments, multi-specific antibodies disclosure herein can include two, three, four, or more domains capable of binding an antigen. Furthermore, multi-specific antibodies can include at least two copies of the same antigen-binding sequence, or two antigen-binding sequences which are specific for different epitopes on the same antigen (biparatopic) as long as the multi-specific antibody has specificity for at least one of OX-40, CD40, 4-1BB, and EFGR and at least one second antigen. In some embodiments the multi-specific antibody (a MVSCA) has specificity for at least two of OX-40, CD40, 4-1BB, HSA, IL-22, and EFGR. In some embodiments, the multi-specific antibodies disclosed herein are single chain antibodies. Accordingly, some multi-specific antibodies can be referred to as antibodies comprising means for binding a first target and means for binding a second target, etc.

“Bispecific antibodies” refers to antibodies which have two different antigen-binding specificities. In some embodiments, bispecific antibodies disclosed herein are specific for two of OX-40, CD40, 4-1BB, HSA, IL-22, and EFGR. Amino acid sequences encoding antigen-binding portions of the bispecific antibodies can be linked in various configurations. In some embodiments, the amino acid sequences encoding the antibody-binding portions of the bispecific antibodies are connected by a linker as disclosed herein.

“Tri-specific antibodies” refers to antibodies which have three different antigen-binding specificities. In some embodiments, the tri-specific antibodies disclosed herein are specific for three of OX-40, CD40, 4-1BB, HSA, IL-22, and EFGR. Amino acid sequences encoding antigen-binding portions of the tri-specific antibodies can be linked in various configurations. In some embodiments, the amino acid sequences encoding the antibody-binding portions of the tri-specific antibodies are connected by a linker as disclosed herein. In some embodiments two linkers are used, which can be the same of different.

“Quadbodies” refers to antibodies which have four different antigen-binding specificities. In some embodiments, the quadbodies disclosed herein are specific for four of OX-40, CD40, 4-1BB, HSA, IL-22, and EFGR. Amino acid sequences encoding antigen-binding portions of the quadbodies can be linked in various configurations. In some embodiments, the amino acid sequences encoding the antibody-binding portions of the quadbodies are connected by a linker as disclosed herein. In some embodiments two linkers are used, which can be the same of different.

The term “valent” as used herein denotes the presence of a specified number of binding sites in an antibody molecule. As such, the terms “bivalent”, “trivalent”, “tetravalent”, “pentavalent”, “hexavalent”, “heptavalent”, and “octavalent” denote the presence of two binding sites, three binding sites, four binding sites, five binding sites, six binding sites, seven binding sites, and eight binding sites, respectively, in an antibody molecule. The bispecific antibodies disclosed herein are “bivalent”. The tri-specific antibodies disclosed herein are “trivalent.” The quadbodies disclosed herein are “tetravalent.” However, monospecific multivalent antibodies, for example, bivalent, trivalent, and tetravalent antibodies, are within the scope of the present disclosure in which the multiple antigen-binding sites bind the same antigen. The antigen-binding sites of monospecific bivalent and trivalent (or higher valency) antibodies can bind either the same epitope or different epitopes on the antigen. Similarly, by combining multiple monospecific binding sites with binding sites for one or more other specificities antibodies can be constructed in which the valency is of a higher order than the multi-specificity, for example, a trivalent, bispecific antibody.

By “full length antibody” herein is meant the structure that constitutes the natural biological form of an antibody, including variable and constant regions. For example, in most mammals, including humans and mice, the full length antibody of the IgG class is a tetramer and consists of two identical pairs of two immunoglobulin chains, each pair having one light and one heavy chain, each light chain comprising immunoglobulin domains VL and CL, and each heavy chain comprising immunoglobulin domains VH, CH1, CH2, and CH3. In some mammals, for example in camels and llamas, IgG antibodies can also consist of only two variable heavy chains, each heavy chain comprising a variable domain (VHH) attached to the Fc region (CH2 and CH3 domains).

Tetrameric antibodies are typically composed of two identical pairs of polypeptide chains, each pair having one “light” (typically having a molecular weight of about 25 kDa) and one “heavy” chain (typically having a molecular weight of about 50-70 kDa). Each of the light and heavy chains are made up of two distinct regions, referred to as the variable and constant regions. For the IgG class of immunoglobulins, the heavy chain is composed of four immunoglobulin domains linked from N-to C-terminus in the order VH-CH1-CH2-CH3, referring to the heavy chain variable domain, heavy chain constant domain 1, heavy chain constant domain 2, and heavy chain constant domain 3 respectively (also referred to as VH-Cγ1-Cγ2-Cγ3, referring to the heavy chain variable domain, constant gamma 1 domain, constant gamma 2 domain, and constant gamma 3 domain respectively). The IgG light chain is composed of two immunoglobulin domains linked from N-to C-terminus in the order VL-CL, referring to the light chain variable domain and the light chain constant domain respectively. The constant regions show less sequence diversity, and are responsible for binding a number of natural proteins to elicit important biochemical events.

The variable region of an antibody contains the antigen-binding determinants of the molecule, and thus determines the specificity of an antibody for its target antigen. The variable region is so named because it is the most distinct in sequence from other antibodies within the same class. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. There are six CDRs total, three each per heavy and light chain, designated VH CDR1, VH CDR2, VH CDR3, VL CDR1, VL CDR2, and VL CDR3. The variable region outside of the CDRs is referred to as the framework (FR) region. Although not as diverse as the CDRs, sequence variability does occur in the FR region between different antibodies. Overall, this characteristic architecture of antibodies provides a stable scaffold (the FR region) upon which substantial antigen-binding diversity (the CDRs) can be explored by the immune system to obtain specificity for a broad array of antigens.

The genes encoding the immunoglobulin locus comprise multiple V region sequences along with shorter nucleotide sequences named “D” and “J” and it is the combination of the V, D, and J nucleotide sequence that give rise to the VH diversity.

Antibodies are grouped into classes, also referred to as isotypes, as determined genetically by the constant region. Human constant light chains are classified as kappa (C_{κ}) and lambda (CA) light chains. Heavy chains are classified as mu (μ), delta (δ), gamma (γ), alpha (α), or epsilon (ϵ), and define the antibody's isotype as IgM, IgD, IgG, IgA, and IgE, respectively. The IgG class is the most commonly used for therapeutic purposes. In humans this class comprises subclasses IgG1, IgG2, IgG3, and IgG4. In mice this class comprises subclasses IgG1, IgG2a, IgG2b, IgG3. IgM has subclasses, including, but not limited to, IgM1 and IgM2. IgA has several subclasses, including but not limited to IgA1 and IgA2. Thus, “isotype” as used herein is meant any of the classes or subclasses of immunoglobulins defined by the chemical and antigenic characteristics of their constant regions. The known human immunoglobulin isotypes are IgG1, IgG2, IgG3, IgG4, IgA1, IgA2, IgM1, IgM2, IgD, and IgE. The disclosed VHH antibodies, bispecific, and multi-specific antibodies can have constant regions comprising all, or part, of the above-described isotypes.

Also within the scope of the present disclosure are antibody fragments including, but are not limited to, (i) a Fab fragment comprising VL, CL, VH, and CH1 domains, (ii) a Fd fragment comprising VH and CH1 domains, (iii) a Fv fragment comprising VL and VH domains of a single antibody; (iv) a dAb fragment comprising a single variable region, (v) isolated CDR regions, (vi) F(ab′)₂ fragment, a bivalent fragment comprising two linked Fab fragments, and (vii) a single chain Fv molecule (scFv), wherein a VH domain and a VL domain are linked by a peptide linker which allows the two domains to associate to form an antigen-binding site. Trivalent or tetravalent antibody fragments comprising variable domains of having three different specificities and linked by cleavable or uncleavable linkers are also disclosed. In certain embodiments, antibodies are produced by recombinant DNA techniques. In additional embodiments, antibodies are produced by enzymatic or chemical cleavage of naturally occurring antibodies.

“Single-chain antibody” as used herein, refers to a fusion protein of the antigen-binding portions of antibodies (i.e., variable regions) generally connected by a linker peptide. Disclosed herein are multivalent mono- and multi-specific single chain antibodies. The monospecific multivalent antibodies have specificity for at least one of OX-40, CD40, 4-1BB, HSA, IL-22, and EFGR. The multi-specific single chain antibodies have specificity for at least one of OX-40, CD40, 4-1BB, HSA, IL-22, and EFGR plus at least one further specificity. In some embodiments, the multi-specific single chain antibodies have specificity for at least two of OX-40, CD40, 4-1BB, HSA, IL-22, and EFGR.

By “humanized” antibody as used herein is meant an antibody comprising a human framework region (FR) and one or more complementarity determining regions (CDR's) from a non-human antibody. The non-human antibody providing the CDR's is called the “donor” and the human immunoglobulin providing the framework is called the “acceptor”. In certain embodiments, humanization relies principally on the grafting of donor CDRs onto acceptor (human) VL or VH frameworks. This strategy is referred to as “CDR grafting”. “Backmutation” of selected acceptor framework residues to the corresponding donor residues is often required to regain affinity that is lost in the initial grafted construct. The humanized antibody optimally also will comprise at least a portion of an immunoglobulin constant region, typically that of a human immunoglobulin, and often will typically comprise a human Fc region. Humanization or other methods of reducing the immunogenicity of nonhuman antibody variable regions may include resurfacing methods. In one embodiment, selection based methods may be employed to humanize and/or affinity mature antibody variable regions, that is, to increase the affinity of the variable region for its target antigen. Other humanization methods may involve the grafting of only parts of the CDRs, including but not limited to methods described in U.S. Pat. No. 6,797,492, incorporated by reference herein for all it discloses regarding CDR grafting. Structure-based methods may be employed for humanization and affinity maturation, for example as described in U.S. Pat. No. 7,117,096, incorporated by reference herein for all it discloses regarding humanization and affinity maturation.

In various embodiments herein, the antibodies are VHH. Camelids (camels, dromedary, and llamas) contain, in addition to conventional heavy and light chain antibodies (2 light chains and 2 heavy chains in one antibody), two-chain antibodies (containing only variant heavy chains). The dimeric antibodies are coded for by a distinct set of VH segments referred to as VHH genes. The VH and VHH are interspersed in the genome (i.e., they appear mixed in between each other). The identification of an identical D segment in a VH and VHH cDNA suggests the common use of the D segment for VH and VHH. Natural VHH-containing antibodies are missing the entire CH1 domain of the constant region of the heavy chain. The exon coding for the CH1 domain is present in the genome but is spliced out due to the loss of a functional splice acceptor sequence at the 5′ side of the CH1 exon. As a result the VDJ region is spliced onto the CH2 exon. When a VHH is recombined onto such constant regions (CH2, CH3), an antibody is produced in which the half-antibody is a single chain instead of a light chain/heavy chain pair (i.e., an antibody of two heavy chains without a light chain interaction). Binding of an antigen is different from that seen with a conventional antibody, but high affinity is achieved the same way, i.e., through hypermutation of the variable region and selection of the cells expressing such high affinity antibodies.

In an exemplary embodiment, the disclosed VHH are produced by immunizing a transgenic mouse in which endogenous murine antibody expression has been eliminated and camelid transgenes have been introduced. VHH mice are disclosed in U.S. Pat. Nos. 8,883,150, 8,921,524, 8,921,522, 8,507,748, 8,502,014, US 2014/0356908, US2014/0033335, US2014/0037616, US2014/0356908, US2013/0344057, US2013/0323235, US2011/0118444, and US2009/0307787, all of which are incorporated herein by reference for all they disclose regarding heavy chain only antibodies and their production in transgenic mice. The VHH mice are immunized and the resulting primed spleen cells fused with a murine myeloma cells to form hybridomas.

In other embodiments, VHH are produced by immunizing llamas with a desired antigen, and isolating sequencing encoding the VHH regions of resulting antigen-binding antibodies. In one embodiment, the VHH are isolated using a phage display library. See, for example, WO 91/17271; WO 92/01047; and WO 92/06204 (each of which is incorporated by reference in its entirety for description of making phage libraries).

Also disclosed herein are multi-specific or multivalent antibodies in which two or more antigen-binding domains are joined in a single fusion protein. Multi-specific antibodies can take many forms including (i) multi-specific Fv fragments; (ii) a heavy chain of a first specificity having associated therewith (or fused thereto) a second VH domain having a second specificity; (iii) tetrameric monoclonal antibodies with a first specificity having associated therewith with a second VH domain having a second specificity, wherein the second VH domain is associated with a first VH domain); (iv) Fab fragments (VH-CH1/VL-CL) of a first specificity having associated therewith a second VH domain with a second specificity. Exemplary Fab fragments include those in which the second VH sequence having the second specificity is associated with the C-terminus or the N-terminus of the first VH domain, or the C-terminus or the N-terminus of the first CH1 or first CL domains. In additional embodiments, VH sequences having a second and/or a third specificity (or more) can be associated with (or fused to) the C-terminus or the N-terminus of the first VH domain, or the C-terminus or the N-terminus of the first CH1 or first CL domains. In various embodiments any of these formats can include at least one of the herein disclosed VHH domains. Examples of configurations of multi-specific antibodies can be found in WO2021/062361, which is incorporated by reference herein for all it discloses regarding configuration of multi-specific antibodies.

Multi-specific or multivalent antibodies may include linker sequences linking a particular antigen-binding domain (such as a VH or VHH) to another antigen-binding domain and which allows for proper folding of the amino acid sequences to generate the desired three-dimensional conformation and antigen-binding profiles. Generally a linker sequence will be a short amino acid sequence that provides sufficient space and flexibility between the domains for them to fold properly. The linker may also cause steric hindrance so as to facilitate binding to the target of each domain. Suitable linkers include, but are not limited to, the linkers of Table 26 (SEQ ID Nos: 84-103), EPKSCD (SEQ ID NO:104), and ASTKGP (SEQ ID NO:105). Further linkers will be known to the person of skill in the art.

Also within the scope of the present disclosure are amino acid sequence variants of the monospecific or multi-specific antibodies disclosed herein. Amino acid sequence variants are prepared by introducing appropriate nucleotide changes into the antibody-encoding DNA, or by peptide synthesis. Such variants include, for example, deletions from, and/or insertions into and/or substitutions of, residues within the amino acid sequences of the antibodies of the examples herein. Any combination of deletion, insertion, and substitution is made to arrive at the final construct, provided that the final construct possesses the desired characteristics. The amino acid changes also may alter post-translational processes of the humanized or variant antibodies, such as changing the number or position of glycosylation sites.

A useful method for identification of certain residues or regions of the antibodies that are preferred locations for mutagenesis is called “alanine scanning mutagenesis”. A residue or group of target residues are identified (e.g., charged residues such as Arg, Asp, His, Lys, and Glu) and replaced by a neutral amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis is conducted at the target codon or region and the expressed antibody variants are screened for the desired activity.

Amino acid sequence insertions include amino- and/or carboxyl-terminal fusions ranging in length from one residue to polypeptides containing a hundred or more residues, as well as intra-sequence insertions of single or multiple amino acid residues. Examples of terminal insertions include an antibody disclosed herein with an N-terminal methionyl residue or the antibody fused to an epitope tag. Other insertional variants of the antibody molecules include the fusion to the N- or C-terminus of the antibody of an enzyme or a polypeptide which increases the serum half-life of the antibody.

Another type of variant is an amino acid substitution variant. These variants have at least one amino acid residue in the antibody molecule removed and a different residue inserted in its place. The sites of greatest interest for substitutional mutagenesis include the hypervariable regions, but FR alterations are also contemplated. Conservative substitutions are shown in Table 1 under the heading of “preferred substitutions”. If such substitutions result in a change in biological activity, then more substantial changes, denominated “exemplary substitutions” in Table 1, or as further described below in reference to amino acid classes, may be introduced and the products screened.

TABLE

| Original | | |
|---|---|---|
| Residue | Exemplary Substitutions | Preferred Substitutions |
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Tyr |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) Hydrophobic: Norleucine, Met, Ala, Val, Leu, Ile;
(2) Neutral hydrophilic: Cys, Ser, Thr;
(3) Acidic: Asp, Glu;
(4) Basic: Asn, Gin, His, Lys, Arg;
(5) Residues that influence chain orientation: Gly, Pro; and (
6) Aromatic: Trp, Tyr, Phe.

Non-conservative substitutions will entail exchanging a member of one of these classes for another class.

Any cysteine residue not involved in maintaining the proper conformation of the monospecific or multi-specific antibodies also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

Another type of substitutional variant involves substituting one or more hypervariable region residues of a parent antibody (e.g., a humanized or camelid antibody). Generally, the resulting variant(s) selected for further development will have improved biological properties relative to the parent antibody from which they are generated. A convenient way for generating such substitutional variants is affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identified hypervariable region residues contributing significantly to antigen-binding. Alternatively, or in addition, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the monospecific or multi-specific antibodies are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of an antibody disclosed herein.

Other modifications of the monospecific or multi-specific antibodies are contemplated. For example, it may be desirable to modify the antibodies with respect to effector function, so as to enhance the effectiveness of the antibody in treating disease, for example. For example cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). Homodimeric antibodies with enhanced anti-tumor activity may also be prepared using heterobifunctional cross-linkers. Alternatively, an antibody can be engineered which has dual Fc regions and may thereby have enhanced complement lysis and ADCC capabilities.

In another embodiment, an antibody may be conjugated to a “receptor” (such streptavidin) for utilization in pretargeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionuclide).

Covalent modifications of the monospecific or multi-specific antibodies are also included within the scope of this disclosure. They may be made by chemical synthesis or by enzymatic or chemical cleavage of the antibody, if applicable. Other types of covalent modifications of the antibodies are introduced into the molecule by reacting targeted amino acid residues of the antibody with an organic derivatizing agent that is capable of reacting with selected side chains or the N- or C-terminal residues. Exemplary covalent modifications of polypeptides are described in U.S. Pat. No. 5,534,615, specifically incorporated herein by reference for all it discloses regarding covalent modifications of polypeptides. An exemplary type of covalent modification of the antibody comprises linking the antibody to one of a variety of nonproteinaceous polymers, e.g., polyethylene glycol, polypropylene glycol, or polyoxyalkylenes, in the manner set forth in U.S. Pat. Nos. 4,640,835, 4,496,689, 4,301,144, 4,670,417, 4,791,192, or 4,179,337.

The monospecific or multi-specific antibodies disclosed herein may be produced by recombinant means. Thus, disclosed herein are nucleic acids encoding the antibodies, expression vectors containing nucleic acids encoding the antibodies, and cells comprising the nucleic acid encoding the antibodies. Methods for recombinant production are widely known in the state of the art and comprise protein expression in prokaryotic and eukaryotic cells with subsequent isolation of the antibody and usually purification to a pharmaceutically acceptable purity. For the expression of the antibodies as aforementioned in a host cell, nucleic acids encoding the antibody sequences are inserted into expression vectors by standard methods. Expression is performed in appropriate prokaryotic or eukaryotic host cells like CHO cells, NSO cells, SP2/0 cells, HEK293 cells, COS cells, PER.C6 cells, yeast, or E. coli cells, and the antibody is recovered from the cells (supernatant or cells after lysis). It is to be understood that any recombinantly-expressed protein requires an initiator methionine (or formyl-methionine) or signal sequence at its N-terminus, depending on the expression system used and whether the protein is expressed in the cytoplasm or secreted. Thus in some embodiments, the herein disclosed protein sequences are modified with such additional amino acids at their N-terminus. In some embodiments such N-terminal sequences are cleaved (in whole or in part) from the fully mature sequence, while in other embodiments they are retained.

Accordingly certain embodiments disclosed herein include a method for the preparation of a monospecific or multi-specific antibody, comprising the steps of a) transforming a host cell with at least one expression vector comprising nucleic acid molecules encoding the antibody; b) culturing the host cell under conditions that allow synthesis of the antibody molecule; and c) recovering said antibody molecule from the culture.

The antibodies are suitably separated from the culture medium by conventional immunoglobulin purification procedures such as, for example, protein A-Sepharose, hydroxylapatite chromatography, gel electrophoresis, dialysis, or affinity chromatography.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included. Where distinct designations are intended, it will be clear from the context.

The term “transformation” as used herein refers to process of transfer of a vectors/nucleic acid into a host cell. If cells without formidable cell wall barriers are used as host cells, transfection can be carried out e.g. by the calcium phosphate precipitation method. However, other methods for introducing DNA into cells such as by nuclear injection or by protoplast fusion may also be used. If prokaryotic cells or cells which contain substantial cell wall constructions are used, e.g. one method of transfection is calcium treatment using calcium chloride.

As used herein, “expression” refers to the process by which a nucleic acid is transcribed into mRNA and/or to the process by which the transcribed mRNA (also referred to as transcript) is subsequently being translated into peptides, polypeptides, or proteins. The transcripts and the encoded polypeptides are collectively referred to as gene product. If the polynucleotide is derived from genomic DNA, expression in a eukaryotic cell may include splicing of the mRNA.

A “vector” is a nucleic acid molecule, in particular self-replicating, which transfers an inserted nucleic acid molecule into and/or between host cells. The term includes vectors that function primarily for insertion of DNA or RNA into a cell (e.g., chromosomal integration), replication of vectors that function primarily for the replication of DNA or RNA, and expression vectors that function for transcription and/or translation of the DNA or RNA. Also included are vectors that provide more than one of the functions as described.

An “expression vector” is a polynucleotide which, when introduced into an appropriate host cell, can be transcribed and translated into a polypeptide. An “expression system” usually refers to a suitable host cell comprised of an expression vector that can function to yield a desired expression product.

The term “host cell” as used herein denotes any kind of cellular system which can be engineered to generate the antibodies disclosed herein. In one embodiment HEK293 cells and CHO cells are used as host cells.

The control sequences that are suitable for prokaryotes, for example, include a promoter, optionally an operator sequence, and a ribosome binding site. Eukaryotic cells are known to utilize promoters, enhancers and polyadenylation signals.

A nucleic acid is “operably linked” when it is placed in a functional relationship with another nucleic acid sequence. For example, DNA for a pre-sequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

Also disclosed herein are isolated nucleic acid encoding the monospecific or multi-specific antibodies, vectors and host cells comprising the nucleic acids, and recombinant techniques for the production of the antibodies.

For recombinant production of the antibodies, the nucleic acid encoding it may be isolated and inserted into a replicable vector for further cloning (amplification of the DNA) or for expression. In some embodiments, the antibody may be produced by homologous recombination, e.g. as described in U.S. Pat. No. 5,204,244, specifically incorporated herein by reference for all it discloses regarding antibody production. DNA encoding the antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence, e.g., as described in U.S. Pat. No. 5,534,615, specifically incorporated herein by reference for all it discloses regarding protein expression.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces. One exemplary E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for monospecific or multi-specific antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated monospecific or multi-specific antibodies are derived from multicellular organisms, including invertebrate cells such as plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture); baby hamster kidney cells (BHK, ATCC CCL 10); Chinese hamster ovary cells/-DHFR (CHO); mouse sertoli cells (TM4); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2).

Host cells are transformed with the above-described expression vectors for monospecific or multi-specific antibody production and cultured in conventional nutrient media modified as appropriate for inducing promoters, selecting transformants, or amplifying the genes encoding the desired sequences.

The host cells used to produce the monospecific or multi-specific antibodies may be cultured in a variety of media. Commercially available media such as Ham's F10, Minimal Essential Medium (MEM), RPMI-1640, and Dulbecco's Modified Eagle's Medium (DMEM) are suitable for culturing the host cells. In addition, U.S. Pat. Nos. 4,767,704; 4,657,866; 4,927,762; 4,560,655; or 5, 122,469; WO 90/03430; WO 87/00195; or U.S. Re. Pat. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains, although Protein A can be used to purify antibody that do not have Fc regions. Protein G is useful for all mouse isotypes and for human γ3. The matrix to which the affinity ligand is attached is most often agarose, but other matrices are available. Mechanically stable matrices such as controlled pore glass or poly (styrenedivinyl) benzene allow for faster flow rates and shorter processing times than can be achieved with agarose. Where the antibody comprises a CH3 domain, the Bakerbond ABX™ resin is useful for purification. Antibodies and antibody fragments disclosed herein can also be synthesized with histidine tags and affinity purified by metal affinity chromatography.

Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

Also disclosed herein are multi-specific single chain antibodies that are cleavable in a tumor microenvironment. In some embodiments, a tumor targeting domain (such as a tumor antigen-binding domain) or other functional domain is cleaved at the linker once the multi-specific single chain antibody reaches the tumor, in order to release the other domain(s) which bring about the therapeutic effect. The tumor microenvironment contains a multitude of proteases capable of cleaving the linkers disclosed herein. Non-limiting examples of tumor proteases include, but are not limited to, matrix metalloproteinases (e.g., MMP1, MMP2, MMP3, MMP7, MMP8, MMP9, MMP12, and MMP14), ADAM (a disintegrin and metalloproteinase; e.g., ADAM10 and ADAM17), a kallikrein-related peptidase (e.g., KLK1, KLK2, KLK3, and KLK6), a cathepsin (e.g., CTS-B, CTS-L, and CTS-S), a urokinase plasminogen activator (uPA), a hepsin (HPN), a matriptase, a legumain, or a dipeptidyl peptidase (e.g., DDP4).

### Antibody Compositions

Also disclosed herein are pharmaceutical compositions comprising a monospecific or multi-specific antibody in which the specificities include OX-40, CD40, 4-1BB, HSA, IL-22, or EFGR. Also disclosed is the use of the antibodies described herein for the manufacture of a pharmaceutical composition. Also disclosed are methods of using the disclosed antibodies and pharmaceutical compositions comprising the antibodies for the treatment of various diseases and disorders.

A pharmaceutical composition is one intended and suitable for the treatment of disease in humans. That is, it provides overall beneficial effect and does not contain amounts of ingredients or contaminants that cause toxic or other undesirable effects unrelated to the provision of the beneficial effect. A pharmaceutical composition will contain one or more active agents and may further contain solvents, buffers, diluents, carriers, and other excipients to aid the administration, solubility, absorption or bioavailability, and or stability, etc. of the active agent(s) or overall composition.

The monospecific or multi-specific antibodies disclosed herein may also be formulated in liposomes. Liposomes containing the antibody are prepared by methods known in the art, such as described in U.S. Pat. Nos. 4,485,045, 4,544,545, and 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibodies can be conjugated to the liposomes via a disulfide interchange reaction.

As used herein, “pharmaceutical carrier” includes any and all solvents, dispersion media, coatings, antibacterial and antifungal agents, isotonic and absorption delaying agents, and the like that are physiologically compatible. Preferably, the carrier is suitable for intravenous, intramuscular, intraocular, intravitreal, subcutaneous, parenteral, spinal or epidermal administration (e.g. by injection or infusion). In some embodiments, the carrier is aqueous.

A composition disclosed herein can be administered by a variety of methods known in the art. As will be appreciated by the skilled artisan, the route and/or mode of administration will vary depending upon the desired results. To administer the disclosed antibodies by certain routes of administration, it may be necessary to associate the antibodies with, or co-administer the antibodies with, a material to prevent its inactivation. For example, the antibodies may be administered to a subject in an appropriate carrier, for example, liposomes, or a diluent. Pharmaceutically acceptable diluents include saline and aqueous buffer solutions. Pharmaceutical carriers include sterile aqueous solutions or dispersions and sterile powders for the extemporaneous preparation of sterile injectable solutions or dispersion. The use of such media and agents for pharmaceutically active substances is known in the art.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intraocular, intravitreal, intradermal, intraperitoneal, transtracheal, subcutaneous, subcuticular, intra-articular, subcapsular, subarachnoid, intraspinal, epidural, and intrasternal injection and infusion.

These compositions may also contain excipients such as preservatives, wetting agents, emulsifying agents, and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some embodiments, the pharmaceutical composition comprising the antibody is a lyophilization cake. The lyophilization cake may further comprise bulking agents, buffers and/or salts, or other excipients, such as described herein. The lyophilized composition can be reconstituted by addition of sterile water or aqueous buffer, for administration to the patient.

Regardless of the route of administration selected, the disclosed antibodies, which may be used in a suitable hydrated form, and/or the pharmaceutical compositions containing the antibodies, are formulated into pharmaceutically acceptable dosage forms by conventional methods known to those of skill in the art.

Actual dosage levels of the active ingredients in the pharmaceutical compositions may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

### Linkers

In many embodiments, the individual binding domains are not joined directly to each other, but have a short amino acid sequence interposed between them, a linker. Examples of linkers are shown in Table 10. The length and sequence of the linker can have substantial effects on the expression level, and structure of the MVSCA, and the binding affinity of the linked domains. The adjustable length linkers L2 and L4 (see Table 10) can be used to optimize the MVSCA in terms of these parameter. Linkers L1, L2, and L4 may be termed non-cleavable linker means, flexible linker means, or flexible, non-cleavable linker means.

When two copies of the same VHH domain are placed adjacent to each other in a MVSCA the frequently interact detrimentally with each other. This can be avoided by interposing a relatively short and rigid linker between the two copies. In some embodiments, the short, rigid linker has the sequence AAA (L3 in Table 10). Such linkers may be termed short, rigid linker means or non-cleavable short, rigid linker means.

When an anti-HSA domain-HSA complex is being used to generate a prodrug with respect to the binding activity of an adjacent binding domain, a cleavable linker should be interposed between the two domains. L11*3 through L11*18 (see Table 10) are examples of cleavable linkers of various lengths and susceptibility to cleavage by different proteases that can be used to optimize the MVSCA in terms of expression level, and structure of the MVSCA, the binding affinity of the linked domains, and cleavage. Linkers L11*3 through L11*18 may be termed cleavable linker means, flexible linker means, or flexible, cleavable linker means.

### MVSCAs

The binding domains and linkers described herein can be combined to create multifunctional MVSCA, adapted for the treatment of particular diseases. They can also be further combined with other binding domains. The MVSCA can also be referred to as comprising means for accomplishing the various functions associated with each component type of binding domain and/or comprising linker means for accomplishing their associated functions.

### Use of the Disclosed Antibodies

The disclosed antibodies are useful in medicine. The terms “treatment” “treating”, etc., refer to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. Various embodiments may specifically include or exclude one or more of these modes of treatment.

Use of the herein disclosed antibodies in diagnostics and imaging is also contemplated.

Further, the term “treating” or “treatment” broadly includes any kind of treatment activity, including the diagnosis, mitigation, or prevention of disease, or aspect thereof, in man or other animals, or any activity that otherwise affects the structure or any function of the body of man or other animals. Treatment activity includes the administration of the medicaments, dosage forms, and pharmaceutical compositions described herein to a patient, especially according to the various methods of treatment disclosed herein, whether by a healthcare professional, the patient his/herself, or any other person. Treatment activities include the orders, instructions, and advice of healthcare professionals such as physicians, physician's assistants, nurse practitioners, and the like, that are then acted upon by any other person including other healthcare professionals or the patient him/herself. This includes, for example, direction to the patient to undergo, or to a clinical laboratory to perform, a diagnostic procedure, such as for cancer diagnosis and staging, so that ultimately the patient may receive the benefit appropriate treatment. In some embodiments, the orders, instructions, and advice aspect of treatment activity can also include encouraging, inducing, or mandating that a particular medicament, or combination thereof, be chosen for treatment of a condition- and the medicament is actually used—by approving insurance coverage for the medicament, denying coverage for an alternative medicament, including the medicament on, or excluding an alternative medicament, from a drug formulary, or offering a financial incentive to use the medicament, as might be done by an insurance company or a pharmacy benefits management company, and the like. In some embodiments, treatment activity can also include encouraging, inducing, or mandating that a particular medicament be chosen for treatment of a condition- and the medicament is actually used—by a policy or practice standard as might be established by a hospital, clinic, health maintenance organization, medical practice or physicians group, and the like. All such orders, instructions, and advice are to be seen as conditioning receipt of the benefit of the treatment on compliance with the instruction. In some instances, a financial benefit is also received by the patient for compliance with such orders, instructions, and advice. In some instances, a financial benefit is also received by the healthcare professional for compliance with such orders, instructions, and advice.

### Cancer

The disclosed monospecific VHH and multivalent single chain antibodies having specificity for OX-40, CD40, 4-1BB, HSA, IL-22, and EFGR are useful for treating cancer. Each antibody is designed for treatment for a specific class of cancers based on the antigen-binding specificities included in the antibody.

The present disclosure provides a method of treating cancer comprising administering to a patient in need of such treatment an effective amount of an antibody disclosed herein or a pharmaceutical composition comprising the antibody.

Examples of cancers which can be treated by the disclosed methods include acute lymphoblastic leukemia; acute myeloid leukemia; adrenocortical carcinoma; AIDS-related lymphoma; AIDS-related malignancies; anal cancer; astrocytoma; bile duct cancer, bladder cancer; bone cancer; brain stem glioma; brain tumor; breast cancer; bronchial adenomas/carcinoids; carcinoid tumor; islet cell carcinoma; carcinoma of unknown primary; central nervous system lymphoma; cerebellar astrocytoma; cerebral astrocytoma/malignant glioma; cervical cancer; chronic lymphocytic leukemia; chronic myelogenous leukemia; chronic myeloproliferative disorders; colon cancer; colorectal cancer; cutaneous T-cell lymphoma; endometrial cancer, ependymoma; ovarian epithelial cancer; esophageal cancer; Ewing's family of tumors; extracranial germ cell tumor; intraocular melanoma; retinoblastoma; gallbladder cancer; gastric cancer; germ cell tumor; gestational trophoblastic tumor; hairy cell leukemia; head and neck cancer; hepatocellular cancer; Hodgkin's lymphoma; hypopharyngeal cancer; Kaposi's sarcoma; kidney cancer; laryngeal cancer; non-small cell lung cancer; small cell lung cancer; non-Hodgkin's lymphoma; Waldenstrom's macroglobulinemia; malignant mesothelioma; malignant thymoma; medulloblastoma; melanoma; Merkel cell carcinoma; squamous neck cancer; multiple endocrine neoplasia syndrome; multiple myeloma/plasma cell neoplasm; mycosis fungoides; myelodysplastic syndromes; nasopharyngeal cancer; neuroblastoma; oral cancer; oropharyngeal cancer; osteosarcoma; pancreatic cancer; parathyroid cancer; penile cancer; pheochromocytoma; pituitary tumor; pleuropulmonary blastoma; prostate cancer; rectal cancer; rhabdomyosarcoma; salivary gland cancer; soft tissue sarcoma; Sezary syndrome; skin cancer; squamous neck cancer; testicular cancer; thymoma; thyroid cancer; trophoblastic tumor; urethral cancer; uterine cancer; vaginal cancer; vulvar cancer; and Wilms' tumor.

The effectiveness of cancer therapy is typically measured in terms of “response.” The techniques to monitor responses can be similar to the tests used to diagnose cancer such as, but not limited to:
A lump or tumor involving some lymph nodes can be felt and measured externally by physical examination.
Some internal cancer tumors will show up on an x-ray or CT scan and can be measured with a ruler.
Blood tests, including those that measure organ function can be performed.
A tumor marker test can be done for certain cancers.

Regardless of the test used, whether blood test, cell count, or tumor marker test, it is repeated at specific intervals so that the results can be compared to earlier tests of the same type.

Response to cancer treatment is defined several ways:
Complete response—all of the cancer or tumor disappears; there is no evidence of disease. Expression level of tumor marker (if applicable) may fall within the normal range.
Partial response—the cancer has shrunk by a percentage but disease remains. Levels of a tumor marker (if applicable) may have fallen (or increased, based on the tumor marker, as an indication of decreased tumor burden) but evidence of disease remains.
Stable disease—the cancer has neither grown nor shrunk; the amount of disease has not changed. A tumor marker (if applicable) has not changed significantly.
Disease progression—the cancer has grown; there is more disease now than before treatment. A tumor marker test (if applicable) shows that a tumor marker has risen.

Other measures of the efficacy of cancer treatment include intervals of overall survival (that is time to death from any cause, measured from diagnosis or from initiation of the treatment being evaluated)), cancer-free survival (that is, the length of time after a complete response cancer remains undetectable), and progression-free survival (that is, the length of time after disease stabilization or partial response that resumed tumor growth is not detectable).

There are two standard methods for the evaluation of solid cancer treatment response with regard to tumor size (tumor burden), the WHO and RECIST standards. These methods measure a solid tumor to compare a current tumor with past measurements or to compare changes with future measurements and to make changes in a treatment regimen. In the WHO method, the solid tumor's long and short axes are measured with the product of these two measurements is then calculated; if there are multiple solid tumors, the sum of all the products is calculated. In the RECIST method, only the long axis is measured. If there are multiple solid tumors, the sum of all the long axes measurements is calculated. However, with lymph nodes, the short axis is measured instead of the long axis.

### Autoimmune Diseases

The disclosed monospecific VHH and multivalent single chain antibodies having specificity for OX-40, CD40, 4-1BB, HSA, IL-22, and EFGR are useful for treating autoimmune diseases. Each antibody is designed for treatment for a specific class of autoimmune diseases based on the antigen-binding specificities included in the antibody.

The present disclosure provides a method of treating autoimmune diseases comprising administering to a patient in need of such treatment an effective amount of an antibody disclosed herein or a pharmaceutical composition comprising the antibody.

The autoimmune disorder can be a systemic autoimmune disorder or an organ-specific autoimmune disorder. Non-limiting examples of an autoimmune disorder that can be treated using a compound, composition, or combination disclosed herein include acute disseminated encephalomyelitis (ADEM), Addison's disease, an allergy, allergic rhinitis, anti-phospholipid antibody syndrome (APS), an arthritis such as, e.g., monoarthritis, oligoarthritis, or a polyarthritis like osteoarthritis, rheumatoid arthritis, juvenile idiopathic arthritis, septic arthritis, spondyloarthropathy, gout, pseudogout, or Still's disease, asthma, acquired immunodeficiency syndrome, acquired immunodeficiency syndrome (AIDS), autoimmune hemolytic anemia, autoimmune hepatitis, autoimmune inner ear disease, bullous pemphigoid, celiac disease, Chagas disease, chronic obstructive pulmonary disease (COPD), diabetes mellitus type 1 (IDDM), endometriosis, a gastrointestinal disorder such as, e.g., an irritable bowel disease or an inflammatory bowel disease like Crohn's disease or ulcerative colitis, a glomerulonephritis, Goodpasture's syndrome, Graves' disease, Guillain-Barre syndrome (GBS), Hashimoto's thyroiditis, hidradenitis suppurativa, idiopathic thrombocytopeniaurpura, interstitial nephritis, interstitial cystitis, a lupus, such as, e.g., discoid lupus erythematosus, drug-induced lupus erythematosus. lupus nephritis, neonatal lupus, subacute cutaneous lupus erythematosus, or systemic lupus erythematosus, morphea, multiple sclerosis (MS), myasthenia gravis, a myopathy such as, e.g., dermatomyositis, inclusion body myositis, or polymyositis, myositis, narcolepsy, neuromyotonia, pemphigus vulgaris, pernicious anaemia, primary biliary cirrhosis, psoriasis, psoriatic arthritis, a pulmonary fibrosis, recurrent disseminated encephalomyelitis, rheumatic fever, schizophrenia, scleroderma, Sjogren's syndrome, a skin disorder such as, e.g., dermatitis, eczema, statis dermatitis, hidradenitis suppurativa, psoriasis, rosacea or scleroderma, tenosynovitis, uveitis, a vasculitis such as, e.g., Buerger's disease, cerebral vasculitis, Churg-Strauss arteritis, cryoglobulinemia, essential cryoglobulinemic vasculitis, giant cell arteritis, Golfer's vasculitis, Henoch-Schonlein purpura, hypersensitivity vasculitis, Kawasaki disease, microscopic polyarteritis/polyangiitis, polyarteritis nodosa, polymyalgia rheumatica (PMR), rheumatoid vasculitis, Takayasu arteritis, Wegener's granulomatosis, or vitiligo.

Aspects of the present disclosure includes, in part, reducing at least one symptom associated with an autoimmune disorder. The actual symptoms associated with an autoimmune disorder disclosed herein are well known and can be determined by a person of ordinary skill in the art by taking into account factors, including, without limitation, the location of the autoimmune disorder, the cause of the autoimmune disorder, the severity of the autoimmune disorder, the tissue or organ affected by the autoimmune, and the inflammation associated with the autoimmune disorder. Non-limiting examples of a symptom reduced by a method of treating an autoimmune disorder disclosed herein include inflammation, fatigue, pain, cognitive deficits, neurologic deficits, dizziness, malaise, elevated fever and high body temperature, extreme sensitivity to cold in the hands and feet, weakness, soreness, and/or stiffness in muscles and joints, weight changes, digestive or gastrointestinal problems, breathing problems, low or high blood pressure, irritability, anxiety, or depression, infertility or reduced sex drive (low libido), blood sugar changes, and depending on the type of autoimmune disease, an increase in the size of an organ or tissue, or the destruction of an organ or tissue. Non-limiting examples of an inflammation symptom reduced by a method of treating an autoimmune disorder disclosed herein include pain, loss of neurologic function, loss of cognitive function, edema, hyperemia, erythema, bruising, tenderness, stiffness, swollenness, fever, a chill, congestion of the respiratory tract including nose, and bronchi, congestion of a sinus, a breathing problem, fluid retention, a blood clot, a loss of appetite, an increased heart rate, a formation of granulomas, fibrinous, pus, or non-viscous serous fluid, a formation of an ulcer, or pain.

In certain embodiments, treatment with an antibody disclosed herein reduces at least one symptom, at least two symptoms, at least three symptoms, at least four symptoms, or at least five symptoms of an autoimmune disorder.

In other embodiments, the method may help to treat or alleviate conditions, symptoms, or disorders related to autoimmune diseases. In some embodiments, these conditions or symptoms may include, but are not limited to, anemia, asthenia, cachexia, Cushing's Syndrome, fatigue, gout, gum disease, hematuria, hypercalcemia, hypothyroidism, internal bleeding, hair loss, mesothelioma, nausea, night sweats, neutropenia, paraneoplastic syndromes, pleuritis, polymyalgia rheumatica, rhabdomyolysis, stress, swollen lymph nodes, thrombocytopenia, Vitamin D deficiency, or weight loss. In other embodiments, the administration of an antibody disclosed herein prolongs the survival of the individual being treated.

In some embodiments of the method, the mammal may experience improvements from the autoimmune disease as a result of treatment with an antibody disclosed herein.

The following examples, sequence listing, and figures are provided to aid the understanding of the present invention, the true scope of which is set forth in the appended claims. It is understood that modifications can be made in the procedures set forth without departing from the spirit of the invention.

### EXAMPLES

### Example 1

### Anti-OX40 VHH Antibodies

### Isolation of Anti-OX40 VHH Antibody From Immunized Llamas

Immunizations. Two llamas were immunized at Abcore Inc (Ramona, CA) following their standard protocols. Recombinant human OX40-llama Fc (extracellular domain (Leu29-Ala216) accession #P43489, SEQ ID NO:1) were mixed with Complete Freund's Adjuvant (day 0) or Incomplete Freund's Adjuvant (following immunizations) (Difco, BD Biosciences). Six subcutaneous injections per llama was performed at 50 μg/dose at biweekly intervals At day 45, serum was collected from llamas immunized with recombinant OX40-llama Fc protein to define antibody titers against recombinant OX40-His by ELISA. In ELISA, 96-well Maxisorp plates (Nunc) were coated with 100 ng/well OX40-His. After blocking and adding diluted sera samples, the presence of anti-OX40 VHH antibodies was demonstrated using antibody titers of anti-sera were determined by ELISA. 96-well Maxisorp plates (Nunc) were coated with 100 ng/well recombinant OX40-His. After blocking and adding diluted sera samples, the presence of anti-OX40 VHH antibodies was demonstrated using Horseradish Peroxidase (HRP)-conjugated goat anti-llama IgG antibody (Invitrogen)

Extracellular domain of human OX40
extracellular domain (Leu29-Ala216)
(Accession #P43489)
SEQ ID NO: 1
LHCVGDTYPSNDRCCHECRPGNGMVSRCSRSQNTVCRPCGPGFYNDVVSS
KPCKPCTWCNLRSGSERKQLCTATQDTVCRCRAGTQPLDSYKPGVDCAPC
PPGHFSPGDNQACKPWTNCTLAGKHTLQPASNSSDAICEDRDPPATQPQE
TQGPPARPITVQPTEAWPRTSQGPSTRPVEVPGGRAVA

Phage library construction and selection. Peripheral blood mononuclear cells were prepared from serum samples of days 45 for llamas immunized with recombinant OX40 llama FC protein using Ficoll-Paque Plus (GE Healthcare) according to the manufacturer's instructions. Total RNA was extracted from the peripheral blood mononuclear cells using RNeasy Midi Kit (Qiagen) following manufacturer instructions and used as starting material for RT-PCR to amplify VHH encoding gene fragments. These fragments were cloned into a house made phagemid vector, allowing production of recombinant phage particles, after infection with helper phage, which display the VHH as gene-Ill fusion proteins on the surface of the phage particles. Phage was prepared according to standard methods and stored after filter sterilization at 4° C. for further use. Phage libraries obtained from llamas were used for selection. In the selection, biotinylated OX40-HIs was incubated with the phage libraries and subsequently captured on streptavidin Dynabeads (Invitrogen). Following extensive washing, bound phages were eluted with either 1 mg/ml trypsin. The output from the selections was rescued in E. coli TG1 cells. Colonies were picked and sequenced.

cDNAs encoding the positive VHH were synthesized with C-terminal His-tag at Atum (DNA2.0, Inc), and transiently transfected in HEK293 cells, and positive VHH were purified by IMAC chromatography for in vitro functional assays.

Bio-Layer Interferometry (BLI) kinetic binding analysis. Bio-Layer Interferometry (BLI), a label-free technology was used for measuring the binding kinetics of human OX40 hFC (R & D systems) with anti-OX40 VHH. Affinity measurements were performed with Gator equipped with Anti-Penta-His capture (HIS1K) biosensor tips. The assay was performed at 30° C. in 1×PBS buffer (Gibco®, PBS pH7.2). Samples were agitated at 1000 rpm. Prior to analysis, sensors were humidified for 15 min. Purified anti-OX40 VHH was tested for its binding capacity with HIS1K sensor tips. Tips were loaded using 20 μg/ml of anti-OX40 VHH. Loading proceeded for 300 s resulting in capture levels of between 1.8 and 2 nm. Human OX40 antigen were prepared for binding analysis by dilution to concentrations of 100, 150, 250, 350 nM in 1×PBS. Association was initiated and monitored for 200 sec, after which tips were transferred to 1×PBS buffer without Factor protein (Gibco, PBS pH 7.2), in order to monitor dissociation. Sensor data was collected throughout the experiments, processed, and analyzed using the Gator data analysis software.

Blocking Assay. The 96-well plate was coated with 100 μL of 1 μg/mL OX40L-mFc prepared in antigen coating buffer overnight at 4° C., then blocked with 2% BSA for 1 hour at 25° C. A series of dilutions of each OX40 VHH were premixed with OX40-hFc 0.025 μg/at 25° C. for 30 min, then transferred into OX40L-mFc coated plate to incubate for 1 hour. The plate was washed with PBST four times, then incubated with Mouse anti-human IgG Fc-HRP at 25° C. for 1 hour. After four times of wash with PBST, the plate was developed with 100 μL of TMB per well for 10 to 20 min in the dark and then stopped by adding 50 μL of Stop Solution. The plate was read by Molecular Devices Microplate reader at 450 nM. ELISA data was analyzed using GraphPad Prisma 9.1.

ELISA assay. The 96 well plate was coated with 100 μl per well of OX40 Fc prepared at 1 μg/ml in coating buffer overnight at 4° C., then blocked with 200 μl per well of 2% BSA for 1 hour at 25° C. followed by two times washing with PBST. OX40 VHH supernatants were made into series of 4-fold dilution from the top concentration of 500 nM and added to OX40 Fc coated plate 100 μl per well for 1 hour incubation at 25° C. After four times washing with PBST, the plate was incubated with detecting antibody anti-his-HRP (1:4000 dilution) 100 μl per well for one hour with shaking at 60 rpm. The plate was developed with 100 μL of TMB per well in the dark and then stopped by 50 μL of stop solution per well. The plate was read at 450 nM by Molecular Devices Microplate Reader. Data was analyzed using GraphPad Prisma 9.1.

Expression and purification of anti-OX40 VHH antibodies. Positive phage colonies from immunized llama phage libraries were sequenced. Amino acid sequences are listed below in Table 2. cDNA sequences based on amino acid sequences below were fused with human Fc and synthesized at Atum (DNA2.0) in pJ607 expression vector. The expression plasmids was transfected into a HEK293 cell line to produce fully recombinant anti-OX40 VHH antibodies. The expressed anti-OX40 VHH were purified by HiTrap protein A column.

TABLE 2
Llama anti-OX40 VHH Sequences

| | |
|---|---|
| pgXX88-1 | EVQLVESGGGLIQVGGSLTLSCVSSGSPFSSNAMGWYRQAPGKQREWLATISSVG |
| (SEQ ID NO: 2) | DYTEYAPSVKGRFTISRDDARNTLYLQMNSLRSEDTAVYYCKRCQTWGFGMCPNT |
| | PEDLWGQGTQVTVSS |
| pgXX88-2 | EVQLVESGGGLVQPGGSLRLSCAASGFAFGDIPMMWVRQAPGKGREWVSSISADS |
| (SEQ ID NO: 3) | ATQSYADSVQDRFTISRDNAKNTLYLEMHILKPEDTAVYYCATTDRPPPGQGTQV |
| | TVSS |
| pgXX88-5 | QVQLVESGGGLVQAGGSLRLSCAASGGTFSTYAMGWFRQAPGKEPQFVARITWSG |
| (SEQ ID NO: 4) | YTLYTEPVQGRFAISRDNAKNTVYLQMNSLKPEDTAEYFCAADSSEFRDIPNRDS |
| | RANYRYWGQGTQVTVSS |
| pgXX88-6 | EVQLVESGGGLVQPGGSLKLSCAASGFRWNYYSIFWFREAPGKEREGISRISGID |
| (SEQ ID NO: 5) | GRAYYADSVKGRFTIFRDSSKSTVYLQMNSLKPEDTARYYCATRPPQYGSTCPIT |
| | PRLYAYRGQGTQVTVSS |
| pgXX88-7 | EVQLVESGGRLVQAGDSLKLSCTASGRTFSDYVMGWFRQVPGEGRLMVAVITPAG |
| (SEQ ID NO: 6) | DTTEYADAVKGRFSISKDSAKSTVNLQMNNLKVDDTAVYYCAAGPSWIGMQFGTA |
| | EYWGQGTQVTVSS |
| pgXX88-8 | QVQLVESGGGLVQAGGSLTLSCVSSGSPFSSNAMGWYRQAPGKQRKWLATVSSVG |
| (SEQ ID NO: 7) | DYTEYAPSVKGRFTISRDDARNTLYLQMNNLRSEDTAVYYCKRCQTWGFGMCPNT |
| | PEDLWGQGTQVTVSS |
| pgXX88-9 | EVQLVESGGGLVQPGGSLRLSCAASGIPFGSTAMGWYRQAPGLERELVACISSVG |
| (SEQ ID NO: 8) | DHTNYADSVKGRFTVSRDNSKNTVYLQMNSLKPEDTAVYYCARGQSWMFGMCPVT |
| | AQDSWGQGTQVTVSS |
| pgXX88-10 | EVQLVESGGGLVQAGESLRLSCSASGSIRLNAVGWFRQAPVNQRLMVATITHNNS |
| (SEQ ID NO: 9) | TNYADSVQGRFTIARDDAKNTVTLQMNSLKPEDTAVYMCKVYQLPVPISSHDDYW |
| | GQGTQVTVSS |
| pgXX88-11 | QVQLVESGGGLVQPGGSLRLSCAASGSTLSFHAMGWFRQVPGKQRELVAAISTVG |
| (SEQ ID NO: 10) | DYTNYASSVEGRFTISRDNAVKVVYLQMNSLKPDDTAVYYCQHCRSWLFGTCPYT |
| | PQDYWGQGTQVTVSS |
| pgXX88-15 | EVQLVESGGDLVQPGGSLTLSCVGRGSPFGTHAMGWYRQIGAKVRELVAAISSGG |
| (SEQ ID NO: 11) | DYTNYADSVKGRFTISRGVTNDTLYLQMNRLKPEDTAVYYCVRCQFWAFGTCPNT |
| | PQDSWSQGTQVTVSS |
| pgXX88-16 | QVQLVESGGGLVQAGESLNLSCSASGSIRNNAVAWFRQGPGNQRLRVAAITNTGT |
| (SEQ ID NO: 12) | TDYADSVQGRFSIARDNAENTVTLQMNSLKPEDTAIYYCKIYTLPVPISSHDDTW |
| | GQGTQVTVSS |
| pgXX88-19 | EVQLVESGGGLVQAGGSLRLSCVATGRTRSSTYIMAWFRQAPGKERDFVAAISFI |
| (SEQ ID NO: 13) | GGSPVYTDSVKGRFTISRVIAENTVHLQMNDLTPEDTALYYCAATDSGPLGPSGP |
| | SKYEYWGQGTQVTVSS |
| pgXX88-20 | QVQLVESGGGLVQPGGSLLLSCAASGFRWDYYSIAWFREAPGKEREGISCISGTD |
| (SEQ ID NO: 14) | DRTYYADSVKDRFTISRDDARKTVYLEMKNLKPEDTAVYYCATGQVVFRPSADDS |
| | DDLFTGDCEYTKWGQGTQVTVSS |
| pgXX88-21 | EVQLVESGGGLVQAGGSLRLSCVATGRTRSSTYIMAWFRQAPGKERDFVAAISFI |
| (SEQ ID NO: 15) | GGSPVYTDSVKGRFTISRVIAENTVHLQMNDLTPEDTALYYCAATDSGPLGPSGP |
| | SKYEYWGQGTQVTVSS |
| pgXX88-25 | QVQLVESGGGLVQAGGSLRLSCVSSGSPFSSNAMGWYRQAPGKQREWLATISSVG |
| (SEQ ID NO: 16) | DYTEYAPSVKGRFTISRDDARNTLTLRMNSLRSEDTAVYYCKRCQTWGFGMCPNT |
| | PEDLWGQGTQVTVSS |
| pgXX88-27 | EVQLVESGGGLVQPGGSVTLSCATSKDTFSRYHMGWFRQAAGKEREFVAAIANTG |
| (SEQ ID NO: 17) | TTTDYADFVKGRFTISRDSAKNTVYLQMDSLKVEDTAMYYCVVGSQFMSYDYWGQ |
| | GTQVTVSS |
| pgXX88-SZ-15 | QVQLVESGGGLVQPGGSLRLSCSPSEGAFKMLTTGWFRQVLGKEREFVSAISWGG |
| (SEQ ID NO: 18) | GATYYADSVKGRFTVSRDNAKNTVYLQMNSLKPEDTAVYYCAVDTGNGVATNIRS |
| | QSRYDYWGQGTQVTVSS |
| pgXX88-SZ-4 | QVQLVESGGGLAQPGSSLRLSCTASGTVFSILSATWFRQAPGKAHEARELVAFIS |
| (SEQ ID NO: 19) | SDGVSMHADSVKGRFTISRDNAASTVYLQMDNLQPEDTAVYFCKLSNSMMSLEYW |
| | GRGTQVTVSS |
| pgXX88-60 | EVQLVESGGGLAQPGGSLRLSCAASGFTFSNYAMGWYRQAPGKEREFVAAIDWST |
| (SEQ ID NO: 20) | NALLYGDSVKGRFTISRDNAKNTVFLQMSGLKPEDTALYYCAADRAYHTVVLGPP |
| | EGMDYWGKGTQVTVSS |
| pgXX88-81 | EVQLVESGGGLAQPGSSLRLSCTASGTVFSILSATWFRQAPGKAHEARELVAFIS |
| (SEQ ID NO: 21) | SDGVSMHADSVKGRFTISRDNAKSTVYLQMDNLQPEDTAAYYCKLSNSMMGMEYW |
| | GRGTQVTVSS |
| pgXX88-83 | EVQLVESGGGLVQTGDSLRLSCAALGGTPSTYAIGWFRQAAGKELQFVARITWNG |
| (SEQ ID NO: 22) | DTWYADALPGRVAISRDNAKKTVYLQMNNLKPEDTAEYICAADVSEFRDIPNRDS |
| | RANYRYWGQGTQVTVSS |
| pgXX88-84 | EVQLVESGGRLVQAGDSLKLSCTASGRTFSDYVMGWFRQVPGEGRLMVAVITPAG |
| (SEQ ID NO: 23) | DTTEYADAVKGRFSISKDSAKRTVNLQMNNLKVDDTAVYYCAAGPSWIGMQFGTA |
| | EYWGQGTQVTVSS |
| pgXX88-88 | EVQLVESGGGSVQSGGSLTLSCVASGSTLSTVAMAWFRQPPGKELQFVARIATNG |
| (SEQ ID NO: 24) | YTYYAGGYTYYGIGDASQGRFTISRDDAKNTVYLQMNNLELEDTAVYYCAGDISE |
| | FRDIPNRDSRANYYYWGQGTQVTVSS |
| pgXX88-101 | QVQLVESGGGLMQPGGSLRLSCAASGGPLSSYAMAWFRQAPGKEREFVTFISYNG |
| (SEQ ID NO: 125) | GRIFYADSVKGRFTISRDNAKNTVSLQMNSLKPEDTAVYYCAGGLRSSNYVKPSQ |
| | YNYWGRGTQVTVSS |
| pgXX88-167 | EVQLVESGGSLVQPGGSLRLSCAASGFNFNIYWMDWVRQAPGKGLEWISGISSSG |
| (SEQ ID NO: 126) | GTTNYADSVKGRFTISRDNAKNTLYLQMNSLKSEDTAVYYCARSSGLWYGMDSWG |
| | KGTQVTVSS |
| pgXX88-37 | EVQLVESGGGLVQPGGSLRLSCVASGFTFSNYWMNWVRQAPGKGLEWVSTINTGG |
| (SEQ ID NO: 127 | GSTTYTDSVKGRFTISRDNAKNTVYLQMNSLKPEDTALYYCNAPRWGTSREDDFW |
| | GQGTQVTVSS |
| pgXX88-38 | QVQLVESGGGLVQTGGSLRLSCEASGFDFSNSPMIWVRQAPGKGREWVSAISGDS |
| (SEQ ID NO: 128) | RTQSYSDSVQDRFTISRDNTKSTLYLHMHSLKPEDTAVYYCSKTDDDPPGQGTQV |
| | TVSS |
| pgXX88-41 | EVQLVESGGGLVQPGGSVTLSCATSRDTFSRYHMGWFRQAVGKEREFVAAIARTG |
| (SEQ ID NO: 129) | TTRDYADFVKGRFTISRDSAKNTVYLQMDSLKVEDTAMYYCVVGSQFMSYDYWGQ |
| | GTQVTVSS |
| pgXX88-42 | EVQLVESGGGLVQPGGSLRLSCAASGIPFGSTAMGWYRQAPGLERELVACISSVG |
| (SEQ ID NO: 130) | DHTNYASSVKGRFTISRDNSKDTVYLQMNSLKPEDTAVYYCARGQGWVFGMCPVA |
| | AQDSWGQGTQVTVSS |
| pgXX88-50 | QVQLVESGGGLVQAGESLRLSCSASGSIRLNAVGWWRQGPGDQRLMVATITHNGD |
| (SEQ ID NO: 131) | TNYADSVQGRFTIARDDDKNTVTLQMNSLRPDDTAVYWCKVYQLQVPISSHDDSW |
| | GQGTQVTVSS |
| pgXX88-235 | QVQLVESGGGSVQAGGSLRLSCVASGSTAGVNVMGWYRQAPGNQRDMVASFSFGG |
| (SEQ ID NO: 132) | IPNYAEPVKGRFTISRDHSKKNLDLQMNSLLPEDTGVYVCRARRPSGMFGGTWYD |
| | SPTDYWGQGIQVTVSS |
| pgXX88-243 | QVQLVESGGGLVQPGGSLRLSCAASGIPFGRTAMGWYRQAPGLERELVACISSVG |
| (SEQ ID NO: 133) | DHTNYADSVKGRFTISRDNSKNTVSLQMNNLKPEDTAVYYCARGQSWMFGMCPVT |
| | AQDSWGQGTQVTVSS |
| pgXX-245 | QVQLVESGGGSVQAGGSLRLSCEASGSSFVYSYMYWYRQAPGKERDLVASINSGG |
| (SEQ ID NO: 134) | RTNYGDSVKGRFTISRDGAENIMYLQMNSLTPEDTAVYYCKVRRPSGQFGGVWYS |
| | DADEFWGQGTQVTVSS |
| pgXX-257 | QVQLVESGGGLVQPGGSLRLSCSASGFAFSNLPMMWVREAPGKGREWVASISADS |
| (SEQ ID NO: 135) | RTQSYADSVENRFWISRDNAKNALYLQMNRLRPEDTAVYYCTRTDGDPPGQGTQV |
| | TVSS |
| pgXX88-310 | QVQLVESGGGLVQVGGSLTVSCVSSGSPFSSNAMGWYRQAPGKQREWVATISSVG |
| (SEQ ID NO: 136) | DYTEYAPSVKGRFTISRDDARNTLYLQMNSLRSEDTAVYYCKRCQTWGFGMCNTP |
| | EDLWGQGTQVTVSS |
| pgXX88-269 | pgQVQLVESGGGLVQPGGSLRLSCAASGIPFGSIAMGWYRQAPGLERALVACISS |
| (SEQ ID NO: 137) | AGDHTNYASSVKGRFTISRDNSKDTVYLQMNSLKPEDTAVYYCARGQGWVFGMCP |
| | VTAQDSWGQGTQVTVSS |
| pgXX88-270 | QVQLVESGGGLVQAGGSLRLSCATSGVPFSSNAMGWYRQAPGKERELVAVISSGG |
| (SEQ ID NO: 138) | DYTNYADSVKGRFTISRVMNNNTLYLQMNSLKPEDTAVYYCARCQSWRFGTCPVT |
| | AQDSWGQGTQVTVSS |
| pgXX88-272 | QVQLVESGGGLVQPGGSLRLSCAASGSTLSFHAMGWFRQAPGKERELVAAISTVG |
| (SEQ ID NO: 139) | DYTNYASSVEGRFTISRDNAMKVVYLQMDSLKPDDTAVYYCQHCRSWLFGSCPYT |
| | PQDYWGQGTQVTVSS |
| pgXX88-274 | QVQLVESGGGLVQPGGSLRLSCAASGSTLSFHAMGWFRQVPGKQRELVAAISTVG |
| (SEQ ID NO: 140) | DYTNYASSVEGRFTISRDNAVKVVYLQMNSLKPDDTAVYYCQHCRSWLFGTCPYT |
| | PQDYWGQGTQVTVSS |
| pgXX88-282 | QVQLVESGGGVVEPGQSLRLSCSASENIRLNAVGWWRQGPGNQRLMVATITHNNS |
| (SEQ ID NO: 141) | TNYGDSVQGRFTIARDDAKSTVTLQMNSLKPEDTAVYYCKIYQLPVPISSHDDYW |
| | GQGTQVTVSS |
| pgXX88-283 | QVQLVESGGGSVQAGGSLRLSCVASGSTAGVNVMGWYRQAPGNQRDMVASFSFGG |
| (SEQ ID NO: 142) | IPNYAEPVKGRFAISRDNSRKNLDLQMNSLLPEDTGVYVCRARRPSGMFGGTWYD |
| | SPTDYWGQGIQVTVSS |
| pgXX88-297 | QVQLVESGGGLVQAGESLRLSCSASGNIRLNAVGWFRQGPGNQRLMVATITHNNS |
| (SEQ ID NO: 106) | TNYADSVQGRFTIARDDAKNTVTLQMNSLKPEDTAVYYCKVYELPVPISSHDDDW |
| | GQGTQVTVSS |

The VHH of Table 2 constitute means for binding OX-40.

The results of an ELISA assay of OX40 VHH antibodies against recombinant human OX40-Fc is presented in Table 3.

Bio-Layer Interferometry (BLI) binding analysis of anti-OX-40 VHH molecules is presented in Table 4.

TABLE 3
ELISA Assay of OX40 VHH antibodies against rh-OX40-Fc antigen

| VHH | pgxx88- | pgxx88- | pgxx88- | pgxx88- | pgxx88- | pgxx88- | pgXX88- |
|---|---|---|---|---|---|---|---|
| (nM) | 2 | 5 | 10 | 16 | 19 | 27 | SZ-4 |
| 1000 | 1.667 | 1.092 | 1.623 | 1.611 | 1.685 | 1.387 | 1.776 |
| 200 | 1.658 | 0.543 | 1.606 | 1.61 | 1.624 | 1.298 | 1.583 |
| 40 | 1.632 | 0.242 | 1.538 | 1.479 | 1.615 | 1.354 | 1.493 |
| 8 | 1.634 | 0.103 | 1.477 | 1.523 | 1.54 | 1.336 | 1.12 |
| 1.6 | 1.461 | 0.065 | 1.463 | 1.404 | 1.381 | 1.019 | 0.463 |
| 0.32 | 0.719 | 0.058 | 0.993 | 0.992 | 0.717 | 0.334 | 0.126 |
| 0.064 | 0.183 | 0.057 | 0.369 | 0.447 | 0.282 | 0.115 | 0.076 |
| 0.0128 | 0.077 | 0.057 | 0.119 | 0.169 | 0.126 | 0.155 | 0.062 |

| VHH | pgXX88- | pgXX88- | pgXX88- | pgXX88- | pgXX88- | pgXX88- |
|---|---|---|---|---|---|---|
| (nM) | SZ-15 | 60 | 81 | 83 | 84 | 88 |
| 1000 | 2.121 | 0.713 | 0.772 | 0.504 | 0.34 | 0.567 |
| 200 | 1.938 | 0.61 | 0.666 | 0.243 | 0.296 | 0.525 |
| 40 | 1.947 | 0.718 | 0.75 | 0.252 | 0.324 | 0.594 |
| 8 | 1.894 | 0.668 | 0.772 | 0.183 | 0.31 | 0.591 |
| 1.6 | 1.663 | 0.641 | 0.743 | 0.147 | 0.29 | 0.603 |
| 0.32 | 0.736 | 0.596 | 0.617 | 0.072 | 0.175 | 0.581 |
| 0.064 | 0.166 | 0.284 | 0.262 | 0.063 | 0.085 | 0.348 |
| 0.0128 | 0.068 | 0.057 | 0.06 | 0.05 | 0.057 | 0.064 |

TABLE 4
Determination of OX40 VHH antibodies binding
affinity (KD) using BLI technology

| VHH | Koff(1/s) | Kon(1/Ms) | KD(M) |
|---|---|---|---|
| pgXX88-SZ-4 | 8.73E−05 | 4.72E+05 | 1.85E−10 |
| pgXX88-SZ-15 | 2.59E−04 | 5.39E+05 | 4.80E−10 |
| pgXX88-2 | 2.30E−04 | 4.74E+05 | 4.85E−10 |
| pgXX88-10 | 1.00E−06 | 6.62E+05 | <E−12 |
| pgXX88-60 | 1.00E−06 | 4.64E+05 | <E−12 |
| pgXX88-81 | 1.00E−06 | 5.49E+05 | <E−12 |
| pgXX88-83 | 2.42E−05 | 4.60E+05 | 5.26E−11 |
| pgXX88-84 | 1.00E−06 | 4.94E+05 | <E−12 |
| pgXX88-88 | 1.00E−06 | 4.83E+05 | <E−12 |

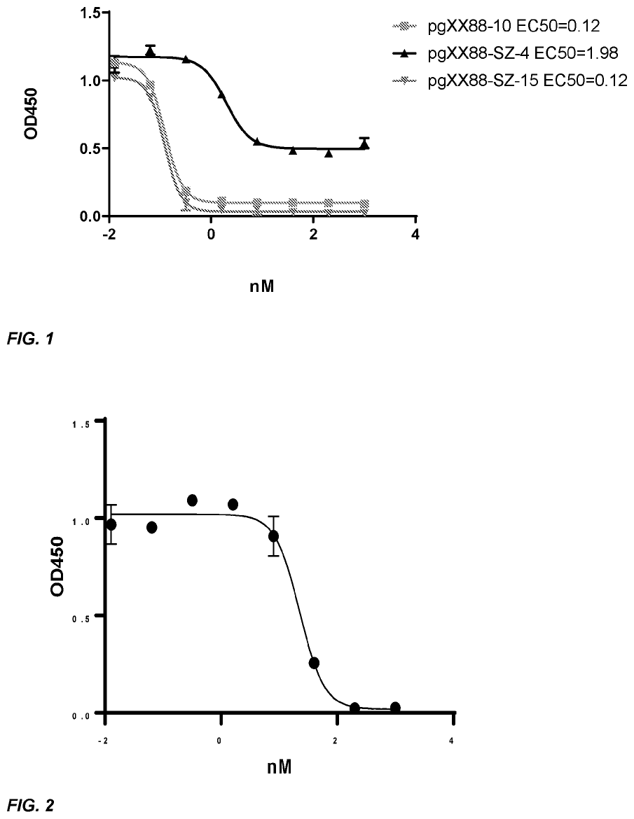

Results of an ELISA blocking assay of the anti-OX40 VHH blocking OX40 binding to OX40 ligand is presented in FIG. 1. pgXX88-10 and pgXX88-SZ-15 are full blockers with EC₅₀ 0.12 nM and 0.12 nM respectively, ppXX88-SZ-4 is a partial blocker with EC₅₀ 1.98 nM.

### Example 2

### Anti-CD40 VHH Antibodies

### Isolation of Anti-CD40 VHH Antibodies From Immunized Llamas

Two llamas were immunized at Abcore Inc. following their standard protocols. Recombinant CD40-llama Fc (extracellular domain (Glu21-Arg193) Accession #P25942-1 in house produced, SEQ ID NO:25) were mixed with Complete Freund's Adjuvant (day 0) or Incomplete Freund's Adjuvant (following immunizations) as in Example 1.

At day 45, serum was collected from llamas immunized with recombinant CD40-llama Fc protein to define antibody titers against recombinant CD40-His by ELISA. In ELISA, 96-well Maxisorp plates were coated with 100 ng/well CD40-His. After blocking and adding diluted sera samples, the presence of anti-CD40 VHH antibodies was demonstrated using antibody titers of anti-sera were determined by ELISA. 96-well Maxisorp plates were coated with 100 ng/well recombinant CD40-His. After blocking and adding diluted sera samples, the presence of anti-CD40 antibodies was demonstrated using HRP-conjugated goat anti-llama IgG antibody.

Extracellular domain of human CD40 extracellular
domain (Glu21-Arg193) (Accession # P25942-1)
SEQ ID NO: 25
EPPTACREKQYLINSQCCSLCQPGQKLVSDCTEFTETECLPCGESEFL
DTWNRETHCHQHKYCDPNLGLRVQQKGTSETDTICTCEEGWHCTSEAC
ESCVLHRSCSPGFGVKQIATGVSDTICEPCPVGFFSNVSSAFEKCHPW
TSCETKDLVVQQAGTNKTDVVCGPQDRLR

Peripheral blood mononuclear cells were prepared from serum samples of days 45 for llamas immunized with recombinant CD40 llama Fc protein using Ficoll-Paque Plus according to the manufacturer's instructions. Total RNA was extracted from the peripheral blood mononuclear cells using RNeasy Midi Kit following manufacturer instructions and used as starting material for RT-PCR to amplify VHH encoding gene fragments. These fragments were cloned into a house made phagemid vector, allowing production of recombinant phage particles, after infection with helper phage, which display the VHH as gene-Ill fusion proteins on the surface of the phage particles. Phage was prepared according to standard methods and stored after filter sterilization at 4° C. for further use. Phage libraries obtained from llamas were used for selection. In the selection, biotinylated CD40-His was incubated with the phage libraries and subsequently captured on streptavidin Dynabeads. Following extensive washing, bound phages were eluted with 1 mg/ml trypsin. The output from the selections was rescued in E. coli TG1 cells. Colonies were picked and sequenced.

cDNAs encoded the positive VHH were synthesized with C-terminal His-tag at Atum, and transiently transfected in HEK293 cells, and positive VHH were purified by IMAC chromatography for in vitro functional assay.

Positive phage colonies from immunized llama phage libraries were sequenced. Amino acid sequences are listed below in Table 5. cDNA sequences based on amino acid sequences below were fused with human Fc and synthesized in pJ607 expression vector. The expression plasmids were transfected into a HEK293 cell line to produce fully recombinant anti-CD40 VHH antibodies. The expressed anti-CD40 VHH were purified by HiTrap protein A column.

TABLE 5
Llama anti-CD40 VHH Sequences

| | |
|---|---|
| pgDD40-SZ-1 | QVQLVESGGALAQPGGSLRLSCEASTNVRSVNAMAWYRQGPGKPRELVANITSDD |
| (SEQ ID NO: 26) | ATYYADSVKGRFTLSREGARNTVYLQMNSLKPEDTAVYQCNVHYTSWGGIRRDYW |
| | GRGTQVTVSS |
| pgDD40-SZ-2 | EVQLVESGGALAQPGGSLRLSCEASATIRSVNAMAWYRQGPGKPREMVANITSGD |
| (SEQ ID NO: 27) | TTYYADSVKGRFTLSREGARNTVYLQMNSLKPEDTAVYQCNIHYTSTGGIRRDYW |
| | GRGTQVTVSS |
| pgDD40-SZ-3 | QVQLVESGGGLVQAGDSLRLSCTASGRAFNTYTMAWFRQAPGKERDFVAAISRDG |
| (SEQ ID NO: 28) | TITHYADSVKGRFTTTRDNAKNTMYLQMSSLKYDDTAVYYCAARRVGAVPERESA |
| | YEHWGQGTQVTVSS |
| pgDD40-SZ-5 | QVQLVESGGALAQPGGSLRLSCEASENVRSVNAMAWYRQGPGKPRELVANITSGD |
| (SEQ ID NO: 29) | ATYYADSVKGRFTLSREGARNTVYLQMNSLKPEDTAVYQCNVHYTSWGGIRRDYW |
| | GRGTQVTVSS |
| pgDD40-SZ-7 | QVQLVESGGALAQPGGSLRLSCEASATVRSVNAMAWYRQGPGKPRELVANITSGD |
| (SEQ ID NO: 30) | TTYYADSVKGRFTLSREGARNTVYLQMNSLKPEDTAVYQCNIHYTSTGGIRRDYW |
| | GRGTQVTVSS |
| pgDD40-SZ-12 | EVQLVESGGGLVQPGGSLKLSCAASGFAFSRYTMSWVRQAPGKGLEWVSGIDSGG |
| (SEQ ID NO: 31) | GRTSYINSVKGRFTISRDNAKNTLYLQMNSLKPEDTAVYHCAQGGDSGSYYRTSW |
| | GQGTQVTVSS |
| pgDD40-HZ-1 | QVQLVESGGGLVQAGGSLRLSCAVSGFMVSFNAMGWYRQAPGKQRELVGGISSGG |
| (SEQ ID NO: 32) | ATNYADSVRRRFTIFRDSAKNTVYLQMNSLKPEDTAVYYCNGGGSLSSVYNPHQY |
| | TYWGQGTQVTVSS |
| pgDD40-HZ-15 | EVQLVESGGGLVQAGGSLRLSCAASGFTFDDYAIGWFRQAPGKEREGVSCLSKNV |
| (SEQ ID NO: 33) | GNTYYADSVKGRFTISSDNAKNTVYLQMNSLKPEDTAVYYCAKGSGYGCYDFPRY |
| | AVWGQGTQVTVSS |
| pgDD40-HZ-24 | EVQLVESGGGSVQAGGSLRLSCAASLSTFSNYRMGWFRRAPGKERELVARIEPHG |
| (SEQ ID NO: 34) | VVDYGDSVKGRFTISRDNAKNTVTVYLHMNSLKPEDTAVYYCNALRLISGRYDPL |
| | FSSWGQGTQVTVSS |
| pgDD40-HZ-28 | EVQLVESGGGLVQAGGSLRLSCVASERTFTPVRMGWFRQAPGKEREIVAAITWSD |
| (SEQ ID NO: 35) | GSTYYADAVKGRFTISRDNALNAVYLQIDSLKPEDTAVYVCAASTSPYSTHLQFR |
| | YWGQGTQVTVSS |
| pgDD40-HZ-47 | QVQLVESGGGLVQPGGSMRLSCSASGFSISVYWMSWVRRAPGKGLEWVSNLGADG |
| (SEQ ID NO: 36) | VTAQYSDSVKGRFTIARDTAKNTLYLQMNSLKPEDTAVYYCNAMTGMNPAEEEAY |
| | WGQGTQVTVSS |
| pgDD40-HZ-58 | QVQLVESGGGLVQPGESLTLSCVTSGFVFSNHWMYWDRQAPGKGLEWVADINLGG |
| (SEQ ID NO: 37) | EMTYYSNSVKGRFTISRDNAKNTVSLRMDNLKVEDTAVYFCAANQQTPPYGLGSR |
| | EYDYDYWGQGTQVTVSS |
| hDD403-4F1 | QVQLVESGGGLVQPGGSLRLSCSASGRAFNTYTMAWFRQAPGKERDFVAAISRDG |
| (SEQ ID NO: 38) | TITYYADSVKGRFTISRDNSKNTLYLQMSSLRAEDTAVYYCAARRVGAVPERESA |
| | YEHWGQGTQVTVSS |
| hDD403-4F1-F | QVQLVESGGGLVQPGGSLRLSCSASGRAFNTYTMAWVRQAPGKGLEYVSAISRDG |
| (SEQ ID NO: 39) | TITYYADSVKGRFTISRDNSKNTLYLQMSSLRAEDTAVYYCAARRVGAVPERESA |
| | YEHWGQGTQVTVSS |
| hDD40-24-F2 | EVQLVESGGGLVQPGGSLRLSCAASLSTFSNYRMGWFRQAPGKERELVARIEPHG |
| (SEQ ID NO: 40) | VVYYADSVKGRFTISRDNAKNTVTVYLQMNSLRAEDTAVYYCNALRLISGRYDPL |
| | FSSWGQGTQVTVSS |

The VHH of Table 5 constitute means for binding CD40.

### ELISA Assay

The 96 well plate was coated with 100 μl per well of CD40 Fc prepared at 1 μg/ml in coating buffer overnight at 4° C., then blocked with 200 μl per well of 2% BSA for 1 hour at 25° C. followed by two times washing with PBST. CD40 VHH supernatants were made into series of 4-fold dilution from the top concentration of 500 nM and added to CD40 Fc coated plate 100 μl per well for 1 hour incubation at 25° C. After four times washing with PBST, the plate was incubated with detecting antibody anti-his-HRP (1:4000 dilution) 100 μl per well for one hour with shaking at 60 rpm. The plate was developed with 100 μL of TMB per well in the dark and then stopped by 50 μL of stop solution per well. The plate was read at 450 nM by Molecular Devices Microplate Reader. Data was analyzed using GraphPad Prisma 9.1 and the results are presented in Table 6.

TABLE 6
ELISA Assay of CD40 VHH antibodies against rh-CD40-Fc antigen

| (nM) | 1000 | 200 | 40 | 8 | 1.6 | 0.32 | 0.064 | 0.013 | 0.0026 |
|---|---|---|---|---|---|---|---|---|---|
| pgDD40-HZ-15 | 0.479 | 0.166 | 0.076 | 0.061 | 0.062 | 0.060 | 0.062 | 0.063 | 0.064 |
| pgDD40-HZ-24 | 1.809 | 1.845 | 1.710 | 1.702 | 0.903 | 0.251 | 0.084 | 0.055 | 0.078 |
| pgDD40-HZ-28 | 0.419 | 0.123 | 0.067 | 0.059 | 0.062 | 0.066 | 0.070 | 0.075 | 0.061 |
| pgDD40-HZ-47 | 0.569 | 0.126 | 0.066 | 0.063 | 0.056 | 0.063 | 0.053 | 0.055 | 0.060 |
| pgDD40-SZ-1 | 1.675 | 1.688 | 1.723 | 1.657 | 0.866 | 0.248 | 0.074 | 0.056 | 0.055 |
| pgDD40-SZ-2 | 1.638 | 1.524 | 1.718 | 1.586 | 0.940 | 0.293 | 0.091 | 0.062 | 0.058 |
| pgDD40-SZ-3 | 1.567 | 1.643 | 1.639 | 1.595 | 1.024 | 0.287 | 0.098 | 0.055 | 0.055 |
| pgDD40-SZ-5 | 1.668 | 1.733 | 1.681 | 1.408 | 0.850 | 0.250 | 0.085 | 0.061 | 0.059 |
| pgDD40-SZ-7 | 1.673 | 1.625 | 1.665 | 1.602 | 1.006 | 0.332 | 0.088 | 0.092 | 0.056 |
| pgDD40-SZ-12 | 1.542 | 0.895 | 0.256 | 0.090 | 0.061 | 0.059 | 0.065 | 0.058 | 0.060 |

### Octet® Kinetic Binding Analysis

Octet® kinetic binding analysis was conducted as in Example 1. Briefly, purified anti-CD40 VHH was tested for its binding capacity with HIS1K sensor tips. Tips were loaded using 20 μg/ml of anti-CD40 VHH. Loading proceeded for 300 sec resulting in capture levels of between 1.8 and 2 nm. Human CD40 antigen were prepared for binding analysis by dilution to concentrations of 100, 150, 250, 350 nM in 1×PBS. Association was initiated and monitored for 200 sec, after which tips were transferred to 1×PBS buffer without CD40 protein in order to monitor dissociation. The results are presented in Table 7.

TABLE 7
Binding Affinity (KD) Analysis of CD40 VHH

| | Koff(1/s) | Kon(1/Ms) | KD(M) |
|---|---|---|---|
| DD40-SZ-1 | 1.00E−06 | 7.79E+05 | <E−12 |
| DD40-SZ-2 | 3.59E−06 | 7.65E+05 | 4.70E−12 |
| DD40-SZ-3 | 7.65E−05 | 7.22E+05 | 1.06E−10 |
| DD40-SZ-5 | 1.00E−06 | 7.84E+05 | <E−12 |
| DD40-SZ-7 | 1.00E−06 | 7.21E+05 | <E−12 |
| DD40-SZ-12 | 8.06E−04 | 5.64E+05 | 1.43E−09 |
| pgDD40-HZ-15 | 1.10E−03 | 3.51E+05 | 3.12E−09 |
| pgDD40-HZ-24 | 1.00E−05 | 6.07E+05 | <E−12 |
| pgDD40-HZ-28 | 3.72E−03 | 9.17E+05 | 4.06E−09 |
| pgDD40-HZ-47 | 6.25E−04 | 1.33E+06 | 4.69E−10 |
| pgDD40-HZ-58 | 3.32E−05 | 8.18E+05 | 4.06E−11 |

### Blocking Assay

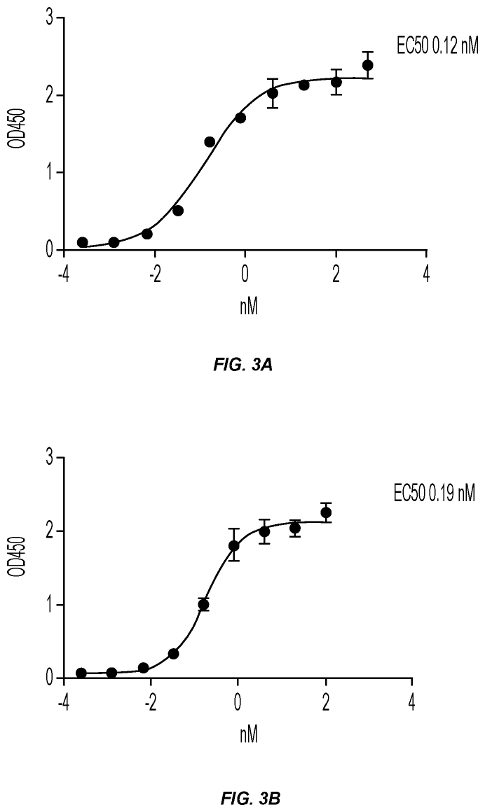

The 96-well plate was coated with 100 μL of 1 μg/mL CD40L-mFc prepared in antigen coating buffer overnight at 4° C., then blocked with 2% BSA for 1 hour at 25° C. A series of dilutions of each CD40 VHH were premixed with CD40-hFc 0.025 μg/at 25° C. for 30 min, then transferred into CD40L-mFc coated plate to incubate for 1 hour. The plate was washed with PBST four times, then incubated with Mouse anti-human IgG Fc-HRP at 25° C. for 1 hour. After four times of wash with PBST, the plate was developed with 100 μL of TMB per well for 10 to 20 min in the dark and then stopped by adding 50 μL of Stop Solution. The plate was read by Molecular Devices Microplate reader at 450 nM. ELISA data was analyzed using GraphPad Prisma 9.1. Results are presented in FIG. 2 and demonstrate that pgDD40-HG-24 is a CD40 receptor blocker.

### Example 3

### Anti-4-1BB VHH Antibodies

### Isolation of Anti-4-1BB VHH Antibodies

Llamas were immunized at Abcore, Inc. with recombinant human 41BB (extracellular domain (Leu24-His183) Accession #Q07011, SEQ ID NO:41) mixed with Complete Freund's Adjuvant (day 0) or Incomplete Freund's Adjuvant (following immunizations) as in Example 1.

Extracellular domain of human 41BB (extracellular
domain (Leu24-His183) (Accession # Q07011)
SEQ ID NO:41
LQDPCSNCPAGTFCDNNRNQICSPCPPNSFSSAGGQRTCDICRQCKGVF
RTRKECSSTSNAECDCTPGFHCLGAGCSMCEQDCKQGQELTKKGCKDCC
FGTFNDQKRGICRPWTNCSLDGKSVLVNGTKERDVVCGPSPADLSPGAS
SVTPPAPAREPGH

4-1BB-binding phage colonies from llama phage libraries were sequenced and the amino acid sequences were listed below (Table 8) for each VHH. cDNA sequences based on amino acid sequences below were synthesized in a pJ607 expression vector. The expression plasmids was transfected into a HEK293 cell line to produce recombinant single domain antibodies (sdAb) with C-terminal his-tag. The expressed sdAb were purified by His Trap HP column.

cDNAs encoding the HSA-specific VHH were synthesized with C-terminal His-tag and transiently transfected in HEK293 cells, and positive VHH were purified by IMAC chromatography.

TABLE 8
Llama anti-4-1BB VHH Sequences

| | |
|---|---|
| 4-1BB-1 | QVQLVESGGGLVQAGGSLRLSCAASENI |
| (SEQ ID NO: 42) | FSFNAMAWYRQAPGKQRELVALITGDGT |
| | TKYADSVKGRFTIARLNAKNTVNLEMNS |
| | LKPEDTAVYTCAALRNSDRTSWGQGTQV |
| | TVSS |
| 4-1BB-2 | QVQLVESGGGLVQAGGSLRLSCAASGSI |
| (SEQ ID NO: 43) | FSYNAMAWYRQAPGKQRELVALITSDRT |
| | TKYADSVKGRFTISRDDTKNTVDLEMNS |
| | LKPEDTAVYTCAALLNSARTSWGQGTQV |
| | TVSS |
| 4-1BB-3 | EVQLVESGGGLVQAGGSLRLSCAASGNI |
| (SEQ ID NO: 44) | FSYNGMAWYRQAPGNQRELVAVVAIDGT |
| | AKYADTVKGRFTISRDNIKNTVNLEMNS |
| | LKPEDTAVYTCAALRNSGSTSWGQGTQV |
| | TVSS |
| 4-1BB-9 | QVQLVESGGGLVQAGGSLRLSCAASGSI |
| (SEQ ID NO: 45) | FSYNAVAWYRQAPGKQRELVAVITSGGE |
| | TTKYADSVKGRFTISRDITKNAVDLQMV |
| | SLKPEDTAVYTCAALRNSGSTSWGQGTQ |
| | VTVSS |
| 4-1BB-10 | EVQLVESGGGLVQAGGSLRLSCAASGAI |
| (SEQ ID NO: 46) | FSYNGMAWYRQAPGKQREFVAVITPDGT |
| | TKYGDSVKGRFTISRDITKNTVDLEMNS |
| | LKPEDTAVYTCAALRNSGSTSWGQGTQV |
| | TVSS |
| 4-1BB-36 | EVQLVESGGGLVQPGGSLRLSCAASRRT |
| (SEQ ID NO: 47) | FSSYTAGWFRQAPGKEREFVASIRWISG |
| | TTTYADSVNGRFTISRDIAKNTVYLQMN |
| | SLKPDDTAVYYCAVDLSSDNDYDYWGQG |
| | TQVTVSS |
| 4-1BB-39 | QVQLGSLGGGSAQPGGSLRLSCTGSGNV |
| (SEQ ID NO: 48) | YDFVVMGWYRQFPGRQRELVANITGRAS |
| | PNYVDSVKGRFTISRDNAQNTVHLQMNN |
| | LKVEDSAIYYCYAAQQLTTIAGVMTLDY |
| | WGSGTQVTVSS |
| 4-1BB-56 | QVQLVESGGGLVQTGGSLRLSCAASGRI |
| (SEQ ID NO: 49) | STIDTMFWYRQTPGKERDWVAFIPKDGL |
| | PTYVDSVKGRFTVSRDNARNTVYLQMNG |
| | LKPEDTAVYYCAVDVQEDRMRTYWGQGT |
| | QVTVSS |
| 4-1BB-59 | EVQLVESGGGLVQPGESLRLSCAASGDT |
| (SEQ ID NO: 50) | VSGYVMLWWRQAPGKERELVAAISSVGY |
| | TYYTDSVKGRFTISRDNAKNTVYLQMNS |
| | LKPEDTAVYYCAADPASISTRSAYWGQG |
| | TQVTVSS |
| h4-1BB56-2 | QVQLVESGGGLVQPGGSLRLSCAASGRI |
| (SEQ ID NO: 51) | STIDTMFWYRQTPGKERDWVAFIPKDGL |
| | PYYADSVKGRFTVSRDNSKNTLYLQMNS |
| | LRAEDTAVYYCAVDVQEDRMRTYWGQGT |
| | QVTVSS |
| h4-1BB56-2F | QVQLVESGGGLVQPGGSLRLSCAASGRI |
| (SEQ ID NO: 52) | STIDTMFWYRQAPGKGLEWVAFIPKDGL |
| | PYYADSVKGRFTISRDNSKNTLYLQMNS |
| | LRAEDTAVYYCAVDVQEDRMRTYWGQGT |
| | QVTVSS |
| h4-1BB59-3 | EVQLVESGGGLVQPGGSLRLSCAASGDT |
| (SEQ ID NO: 53) | VSGYVMLWWRQAPGKERELVSAISSVGY |
| | TYYADSVKGRFTISRDNAKNTLYLQMNS |
| | LRAEDTAVYYCAADPASISTRSAYWGQG |
| | TQVTVSS |

The VHH of Table 4 constitute means for binding 4-1BB.

### BLI Kinetic Binding Analysis

Octet® kinetic binding analysis was conducted as in Example 1. Briefly, purified anti-CD40 VHH was tested for its binding capacity with HIS1K sensor tips. Tips were loaded using 20 μg/ml of anti-41BB VHH. Loading proceeded for 300 sec resulting in capture levels of between 1.8 and 2 nm. Human 41BB antigen were prepared for binding analysis by dilution to concentrations of 100, 150, 250, 350 nM in 1×PBS. Association was initiated and monitored for 200 sec, after which tips were transferred to 1×PBS buffer without 41BB protein, in order to monitor dissociation.

BLI kinetic binding analysis was conducted as in Example 1 and the results are presented in Table 9.

TABLE 9
BLI binding affinity assay of 41BB VHH

| VHH | KD(M) | Koff(1/s) | Kon(1/Ms) |
|---|---|---|---|
| 4-1BB-1 | 3.17E−09 | 2.64E−03 | 8.34E+05 |
| 4-1BB-2 | 2.57E−09 | 2.52E−03 | 9.79E+05 |
| 4-1BB-3 | 1.82E−09 | 1.29E−03 | 7.08E+05 |
| 4-1BB-9 | 4.55E−09 | 2.76E−03 | 6.06E+05 |
| 4-1BB-10 | 6.57E−10 | 5.39E−04 | 8.20E+05 |
| 4-1BB-36 | 2.92E−08 | 9.19E−03 | 3.14E+05 |
| 4-1BB-49 | 2.63E−10 | 3.10E−04 | 1.18E+06 |
| 4-1BB-56 | 7.81E−10 | 5.52E−04 | 7.07E+05 |
| 4-1BB-59 | 2.35E−09 | 1.21E−03 | 5.12E+05 |

### ELISA Assay

The 96 well plate was coated with 100 μl per well of 4-1BB Fc prepared at 1 μg/ml in coating buffer overnight at 4° C., then blocked with 200 μl per well of 2% BSA for 1 hour at 25° C. followed by two rounds of washing with PBST. 4-1BB VHH supernatants were made into a series of 4-fold dilutions from the top concentration of 500 nM and added to 4-1BB Fc-coated plate 100 μl per well for 1 hour incubation at 25° C. After four rounds of washing with PBST, the plate was incubated with detecting antibody anti-his-HRP (1:4000 dilution) 100 μl per well for one hour with shaking at 60 rpm. The plate was developed with 100 μL of TMB per well in the dark and then stopped by 50 μL of stop solution per well. The plate was read at 450 nM by Molecular Devices Microplate Reader. Data was analyzed using GraphPad Prisma 9.1. The results are presented in Table 10.

TABLE 10
ELISA Assay of anti-4-1BB VHH antibodies against rh-41BB

| | 4-1BB- | 4-1BB- | 4-1BB- | 4-1BB- | 4-1BB- | 4-1BB- | 4-1BB- | 4-1BB- |
|---|---|---|---|---|---|---|---|---|
| nM | 1 | 2 | 3 | 9 | 10 | 36 | 39 | 56 |
| 500 nM | 0.547 | 0.112 | 0.165 | 0.264 | 1.578 | 4.248 | 0.098 | 2.699 |
| 50 | 0.308 | 0.129 | 0.183 | 0.292 | 1.759 | 3.185 | 0.065 | 3.079 |
| 5 | 0.108 | 0.070 | 0.089 | 0.084 | 0.828 | 1.349 | 0.070 | 2.894 |
| 0.5 | 0.106 | 0.059 | 0.065 | 0.061 | 0.110 | 0.202 | 0.059 | 1.694 |
| 0.05 | 0.061 | 0.067 | 0.060 | 0.065 | 0.065 | 0.078 | 0.068 | 0.397 |
| 0.005 | 0.064 | 0.063 | 0.067 | 0.064 | 0.066 | 0.068 | 0.064 | 0.203 |
| 0.0005 | 0.065 | 0.058 | 0.062 | 0.067 | 0.068 | 0.071 | 0.072 | 0.181 |
| 0.00005 | 0.067 | 0.065 | 0.066 | 0.070 | 0.068 | 0.073 | 0.063 | 0.129 |

### Blocking Assay

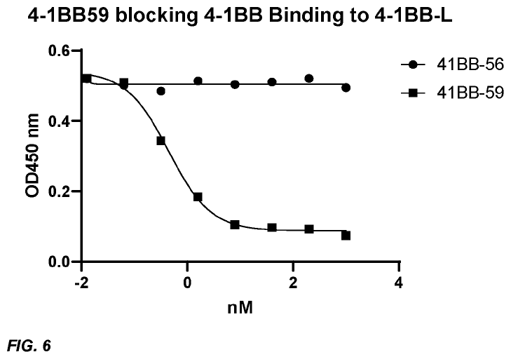

The 96-well plate was coated with 100 μL of 1 μg/mL 41BBL-mFc prepared in antigen coating buffer overnight at 4° C., then blocked with 2% BSA for 1 hour at 25° C. A series of dilutions of each 41BB VHH were premixed with 41BB-hFc 0.025 μg/at 25° C. for 30 min, then transferred into 41BBL-mFc coated plate to incubate for 1 hour. The plate was washed with PBST four times, then incubated with Mouse anti-human IgG Fc-HRP at 25° C. for 1 hour. After four times of wash with PBST, the plate was developed with 100 μL of TMB per well for 10 to 20 min in the dark and then stopped by adding 50 μL of Stop Solution. The plate was read by Molecular Devices Microplate reader at 450 nM. ELISA data was analyzed using GraphPad Prisma 9.1. The results are presented in FIG. 6. 4-1BB59, but not 4-1BB56, blocked 41BB binding to 41BBL in a dose dependent manner.

### Example 4

### Anti-EGFR VHH Antibodies

### Isolation of Anti-EGFR VHH Antibodies

Llamas were immunized at Abcore, Inc. with recombinant human epidermal growth factor receptor (EFGR) (extracellular domain (Met1-Ser645); Accession #CAA25240; SEQ ID NO: 54) mixed with Complete Freund's Adjuvant (day 0) or Incomplete Freund's Adjuvant (following immunizations) and phage libraries prepared as in Example 1.

Human EGFR extracellular domain (Met1-Ser645)
(Accession # CAA25240)
SEQ ID NO: 54
MRPSGTAGAALLALLAALCPASRALEEKKVCQGTSNKLTQLGTFEDHF
LSLQRMFNNCEVVLGNLEITYVQRNYDLSFLKTIQEVAGYVLIALNTV
ERIPLENLQIIRGNMYYENSYALAVLSNYDANKTGLKELPMRNLQEIL
HGAVRFSNNPALCNVESIQWRDIVSSDFLSNMSMDFQNHLGSCQKCDP
SCPNGSCWGAGEENCQKLTKIICAQQCSGRCRGKSPSDCCHNQCAAGC
TGPRESDCLVCRKFRDEATCKDTCPPLMLYNPTTYQMDVNPEGKYSFG
ATCVKKCPRNYVVTDHGSCVRACGADSYEMEEDGVRKCKKCEGPCRKV
CNGIGIGEFKDSLSINATNIKHFKNCTSISGDLHILPVAFRGDSFTHT
PPLDPQELDILKTVKEITGFLLIQAWPENRTDLHAFENLEIIRGRTKQ
HGQFSLAVVSLNITSLGLRSLKEISDGDVIISGNKNLCYANTINWKKL
FGTSGQKTKIISNRGENSCKATGQVCHALCSPEGCWGPEPRDCVSCRN
VSRGRECVDKCKLLEGEPREFVENSECIQCHPECLPQAMNITCTGRGP
DNCIQCAHYIDGPHCVKTCPAGVMGENNTLVWKYADAGHVCHLCHPNC
TYGCTGPGLEGCPTNGPKIPS

For selection of anti-EFGR VHH, biotinylated EFGR-His was incubated with the phage libraries and subsequently captured on streptavidin Dynabeads. Following extensive washing, bound phages were eluted with 1 mg/ml trypsin. The output from the selections was rescued in E. coli TG1 cells. Colonies were picked and sequenced.

cDNAs encoding the EGFR-binding VHH were synthesized with C-terminal His-tag at and transiently transfected in HEK293 cells, and positive VHH were purified by IMAC chromatography.

EGFR-binding phage colonies from immunized llama phage libraries were sequenced and amino acid sequences were listed below (Table 11) for each VHH. cDNA sequences based on amino acid sequences below were fused with human Fc and synthesized in pJ607 expression vector. The expression plasmids was transfected into a HEK293 cell line to produce recombinant anti-EGFR VHH antibodies. The expressed anti-EGFR VHHs were purified by HiTrap protein A column.

Several of the antibodies, pgEG5, pgEG12, pgEG-SX40, and pgEG-SX57, were humanized based on human germline sequences.

TABLE 11
Llama anti-EGFR VHH Sequences

| | |
|---|---|
| pgEG1 | QVQLVESGGGLVQPGGSLRLSCAASGIIFEAEAMGWVRQAPGKQRESVAFIGSGGN |
| (SEQ ID NO: 55) | TNVGWSVKGRFTISRDNAKNTLYLQMNSLRPEDTAVYYCNTHPLRSWGQGTQVTVS |
| pgEG5 | QVQLVESGGGLVQPGGSLRLSCTASHNIFSDNAMAWSRQAPGAQRELVARIATGGN |
| (SEQ ID NO: 56) | TYYPDSVKGRFTISRDNAKNTVYLQMNNLTPDDTAVYYCYAERWTGLEYSRTYWGQ |
| | GTQVTVSS |
| pgEG6 | QVQLVESGGGLVQPGGSLRLSCVASGIIFEAEAMGWYRQAPGKQRESVAFIGSGGN |
| (SEQ ID NO: 57) | TNVGWSVKGRFTISRDNAKNTVYLQMNSLKPEDTAVYYCNTHPLRSWGQGTQVTVS |
| | S |
| pgEG12 | QVQLVESGGGLVQAGGSLSLSCTASRNIFSINVMSWYRQAPGKQREFVASITREDF |
| (SEQ ID NO: 58) | PYYTDSVKGRFTVTRDIANNKVHLQMNSLKLEDTAVYYCNAYYRSDNYWGQGTQVT |
| | VSS |
| pgEG-SX3 | QVQLVESGGGLVQAGGSLRLSCVVYGSIFASTTMGWYRQAPGKQRELVATVYQSGT |
| (SEQ ID NO: 59) | SVYADSVKGRFTASRDNAKKTVYLQMNSLKPEDTAVYYCHPRATDYWGRGTQVTVS |
| | S |
| pgEG-SX6 | QVQLVESGGGTVQAGGSLRLSCVASGMREDIFDMGWYRQAPGLQRELVATITSGGS |
| (SEQ ID NO: 60) | TDYADSVKGRFTISRDNTENTLNLQMNMLKPEDTAVYYCNALYFPPSGGRSSEFWG |
| | QGTLVTVSS |
| pgEG-SX9 | QVQLVESGGGLVQAGGSLRLSCAASVSGSALSNMAWYRQRPGNQRELVAHITFDNF |
| (SEQ ID NO: 61) | TNYADSVKGRFTISRDNVKNTVDLQMSSLKPEDTAVYYCNARHFFGDNYWGKGTLV |
| | TVSS |
| pgEG-SX20 | QVQLVESGGGLVQAGGSLRLSCAGARSAFSIKPMTWYRQAPGKERELVAYFNSGGS |
| (SEQ ID NO: 62) | TNYADSVKGRFTISRDNAKNTMYLQMNNLKPEDTAVYYCNAIPPLGSWGQGTQVTV |
| | SS |
| pgEG-SX22 | QVQLVESGGGLVQAGGSLRVSCAVSGGALSAYAMAWFRQAPGKEREFVAGLNWGGD |
| (SEQ ID NO: 63) | ETYYADSAKGRFTISKDNAKNTVSLQMNSLEPEDTAVYYCGGRPGPLLSRATGYKY |
| | WGQGTQVTVSS |
| pgEG-SX39 | QVQVAESGGGLVQAGESLRLSCTASGSISTINAVRWYRQTPGKQREFVAYISTDGG |
| (SEQ ID NO: 64) | TDYADPVKGRFTISRDNAKNTWFLQMNSLKPEDTGVYMCNVVVTPYAYWGQGTQVT |
| | VSS |
| pgEG-SX40 | QVQLVESGGGLVQAGGSLRLSCTGSGIRFSFYTLGWYRQAPGKQRELVAEIYSSDN |
| (SEQ ID NO: 65) | AKYEDSVKGRFAISRDNAKNSVSLQMNSLKPEDTAVYYCNVRATDYWGPGTQVTVS |
| | S |
| pgEG-SX52 | QVQLVESGGGLVKAGKSLRLSCAASVSSFDTYTMGWYRQAPGKQREQVAIITPGAG |
| (SEQ ID NO: 66) | THYADSVKGRFTISRDNAKNTVYLQMSSLKLEDTAVYYCKARHRITGYDYWGQGTQ |
| | VTVSS |
| pgEG-SX55 | QVQLVESGGGLVQPGGSLRLSCAGTGLIFTTYVMGWYRQAPGKQRELVAIVTNGGG |
| (SEQ ID NO: 67) | THYADFVKGRFTISRDNAKNTVSLQMNLLKPEDTAVYYCNARHLVRPGTNDYWGQG |
| | TLVTVSS |
| pgEG-SX56 | QVQLVESGGGFVQAGGSLRLSCAASGRFLSIADMDWYRRVPGKQRELVASITRAGD |
| (SEQ ID NO: 68) | TALEDSVKGRFTISRDNAKNTVHLQMNSVKPEDTAVYYCRADVTRSGTPYFEVWGQ |
| | GTLVTVSS |
| pgEG-SX57 | QVQLVESGGGLVQAGGSLRLSCAGARSIFSIKPMTWYRQAPGKERELVAWFTSGDS |
| (SEQ ID NO: 69) | PNYADSVKGRFTISRDSAKNTVYLQMNNLKPEDTAVYYCNAVPPLGRWGQGTLVTV |
| | SS |
| hpgEG5 | QAQVQLVESGGGLVQPGGSLRLSCAASHNIFSDNAMAWVRQAPGAQRELVARIATG |
| (SEQ ID NO: 70) | GNTYYADSVKGRFTISRDNAKNTLYLQMNNLRPEDTAVYYCYAERWTGLEYSRTYW |
| | GQGTQVTVSS |
| hpgEG12 | QVQLVESGGGLVQPGGSLRLSCAASHNIFSDNAMAWVRQAPGAQRELVARIATGGN |
| (SEQ ID NO: 71) | TYYADSVKGRFTISRDNAKNTLYLQMNNLRPEDTAVYYCYAERWTGLEYSRTYWGQ |
| | GTQVTVSS |
| hEG40-1 | QVQLVESGGGLVKPGGSLRLSCAASGIRFSFYTMNWVRQAPGKGLEWVSSIYSSDN |
| (SEQ ID NO: 72) | AKYADSVKGRFTISRDNAKNSLYLQMNSLRAEDTAVYYCNVRATDYWGPGTQVTVS |
| | S |
| hEG40-2 | QVQLVESGGGLVQPGGSLRLSCTASGIRFSFYTLGWVRQAPGKQRELVAEIYSSDN |
| (SEQ ID NO: 73) | AKYADSVKGRFTISRDNAKNSVSLQMNSLRAEDTAVYYCNVRATDYWGPGTQVTVS |
| | S |
| hEG40-3 | QVQLVESGGGLVQPGGSLRLSCTASGIRFSFYTLGWVRQAPGKQRELVAEIYSSDN |
| (SEQ ID NO: 74) | AKYADSVKGRFTISRDNAKNSLYLQMNSLRAEDTAVYYCNVRATDYWGPGTQVTVS |
| | S |
| hEG40-4 | QVQLVESGGGLVQPGGSLRLSCTASGIRFSFYTMNWVRQAPGKERELVAEIYSSDN |
| (SEQ ID NO: 75) | AKYADSVKGRFTISRDNAKNSLYLQMNSLRAEDTAVYYCNVRATDYWGPGTQVTVS |
| | S |
| hEG40-5 | QVQLVESGGGLVQPGGSLRLSCTASGIRFSFYTLGWVRQAPGKQRELVSSIYSSDN |
| (SEQ ID NO: 76) | AKYADSVKGRFTISRDNAKNSLYLQMNSLRAEDTAVYYCNVRATDYWGPGTQVTVS |
| | S |
| hEG57-1 | QVQLVESGGGLVQPGGSLRLSCAASRSIFSIKPMSWIRQAPGKERELVAWFTSGDS |
| (SEQ ID NO: 77) | PNYADSVKGRFTISRDNAKNTLYLQMNSLRAEDTAVYYCNAVPPLGRWGQGTLVTV |
| | SS |
| hEG57-2 | QVQLVESGGGLVQPGGSLRLSCAGSRSIFSIKPMSWVRQAPGKERELVAWFTSGDS |
| (SEQ ID NO: 78) | PNYADSVKGRFTISRDNAKNTLYLQMNSLRAEDTAVYYCNAVPPLGRWGQGTLVTV |
| | SS |
| hEG57-3 | QVQLVESGGGLVQpGGSLRLSCAGARSIFSIKPMTWYRQAPGKERELVAWFTSGDS |
| (SEQ ID NO: 79) | PNYADSVKGRFTISRDSAKNTLYLQMNSLRAEDTAVYYCNAVPPLGRWGQGTLVTV |
| | SS |
| hEG57-4 | QVQLVESGGGLVQpGGSLRLSCAGARSIFSIKPMSWVRQAPGKERELVAWFTSGDS |
| (SEQ ID NO: 80) | PNYADSVKGRFTISRDSAKNTLYLQMNSLRAEDTAVYYCNAVPPLGRWGQGTLVTV |
| | SS |
| hEG57-5 | QVQLVESGGGLVQpGGSLRLSCAGARSIFSIKPMTWIRQAPGKERELVAWFTSGDS |
| (SEQ ID NO: 81) | PNYADSVKGRFTISRDSAKNTLYLQMNSLRAEDTAVYYCNAVPPLGRWGQGTLVTV |
| | SS |
| hEG403-F3Y | QVQLVESGGGLVQPGGSLRLSCTASGIRFSFYTLGWYRQAPGKGRELVAEIYSSDN |
| (SEQ ID NO: 82) | AKYADSVKGRFTISRDNAKNSLYLQMNSLRAEDTAVYYCNVRATDYWGPGTQVTVS |
| | S |
| hEG403-F3Y-F | QVQLVESGGGLVQPGGSLRLSCAASGIRFSFYTLGWYRQAPGKGRELVSEIYSSDN |
| (SEQ ID NO: 83) | AKYADSVKGRFTISRDNAKNSLYLQMNSLRAEDTAVYYCNVRATDYWGPGTQVTVS |
| | S |

The VHH of Table 11 constitute means for binding EGFR.

Octet® kinetic binding analysis was conducted as in Example 1 and the K_{D} results are presented in Table 12.

TABLE 12
Binding affinity of anti-EGFR VHH antibodies

| VHH | KD (M) | Kon(1/Ms) | Kdis(1/s) |
|---|---|---|---|
| pgEG1 | 1.56E−08 | 1.56E+05 | 2.43E−03 |
| pgEG5 | 1.08E−08 | 1.24E+05 | 1.34E−03 |
| pgEG6 | 2.79E−08 | 4.89E+05 | 1.36E−02 |
| pgEG12 | 1.25E−08 | 2.01E+05 | 2.51E−03 |
| pgEG-SX-3 | 6.02E−10 | 2.75E−04 | 4.57E+05 |
| pgEG-SX-6 | 7.98E−10 | 3.99E−04 | 5.00E+05 |
| pgEG-SX-22 | 4.64E−10 | 2.80E−04 | 6.02E+05 |
| pgEG-SX-40 | 1.13E−09 | 4.06E−04 | 3.58E+05 |
| pgEG-SX-57 | 1.87E−09 | 7.15E−04 | 3.81E+05 |

### ELISA Assay

The 96 well plate was coated with 100 μl per well of EGFR Fc prepared at 1 μg/ml in coating buffer overnight at 4° C., then blocked with 200 μl per well of 2% BSA for 1 hour at 25° C. followed by two rounds of washing with PBST. pgEG-SX VHH supernatants were made into series of 4-fold dilutions from the top concentration of 500 nM and added to EGFR Fc-coated plate 100 μl per well for 1 hour incubation at 25° C. After four rounds of washing with PBST, the plate was incubated with detecting antibody anti-his-HRP (1:4000 dilution) 100 μl per well for one hour with shaking at 60 rpm. The plate was developed with 100 μL of TMB per well in the dark and then stopped by 50 μL of stop solution per well. The plate was read at 450 nM by Molecular Devices Microplate Reader. Data was analyzed using GraphPad Prisma 9.1. Results of the ELISA assay are in Table 13.

### Blocking Assay

The 96-well plates were coated with 100 μl per well of EGF-mFc prepared at 1 μg/ml (28nM) in coating buffer. After incubation at 4° C. overnight, the plate was washed four times of PBST and blocked with 200 μl per well of 2% BSA for one hour at room temperature. The purified pgEG-SX40 or 57 VHH were made into a series of dilutions from 1000 nM and pre-incubated with 0.05 nM EGFR-hFc at room temperature for 30 min. The plate was washed twice with PBST, and the pre-mixed samples were added to the 96-well plate shaking at 60 rpm for one hour at room temperature. After four washes of PBST, the plate was incubated with detecting antibody anti-his-HRP (1:4000 dilution) 100 μl per well for one hour with shaking at 60 rpm. The plate was washed with four times of PBST and added 100 μl per well of substrate TMB followed by 50 μl of stop solution. The plate was immediately read by ELISA microplate reader at OD₄₅₀ (Table 14).

The purified VHH antibody, SX-40 and SX-57 blocked EGFR binding to EGF, while SX-3 and SX-6 only partially blocked the binding (Table 14).

TABLE 13
ELISA assay of anti-EGFR VHH antibodies

| | pgEG- | pgEG- | pgEG- | pgEG- | pgEG- | pgEG- | pgEG- | pgEG- | pgEG- | pgEG- | pgEG- | |
|---|---|---|---|---|---|---|---|---|---|---|---|---|
| nM | SX3 | SX6 | SX9 | SX20 | SX22 | SX39 | SX40 | SX52 | SX55 | SX56 | SX57 | pgEG5 |
| 500 | 1.467 | 1.368 | 2.203 | 2.529 | 2.164 | 0.503 | 0.963 | 2.225 | 2.238 | 1.495 | 2.611 | 2.515 |
| 50 | 1.205 | 1.147 | 2.277 | 2.674 | 1.777 | 0.147 | 0.786 | 2.325 | 2.203 | 1.473 | 2.463 | 2.33 |
| 10 | 0.994 | 1.004 | 2.396 | 2.602 | 1.942 | 0.093 | 0.715 | 2.438 | 2.418 | 1.541 | 2.543 | 2.349 |
| 2 | 0.843 | 1.83 | 2.639 | 2.607 | 1.88 | 0.073 | 0.626 | 2.339 | 2.218 | 1.46 | 2.786 | 2.228 |
| 0.4 | 0.762 | 1.155 | 2.403 | 2.666 | 1.721 | 0.067 | 0.461 | 1.996 | 1.609 | 1.317 | 2.67 | 1.204 |
| 0.08 | 0.373 | 0.496 | 1.839 | 1.947 | 1.433 | 0.068 | 0.25 | 1.072 | 0.5 | 1.154 | 2.374 | 0.34 |
| 0.016 | 0.134 | 0.139 | 0.903 | 1.131 | 0.871 | 0.072 | 0.144 | 0.32 | 0.193 | 0.569 | 1.332 | 0.147 |
| 0.0032 | 0.086 | 0.082 | 0.237 | 0.358 | 0.304 | 0.075 | 0.121 | 0.196 | 0.125 | 0.409 | 0.648 | 0.119 |
| EC₅₀ | 0.3818 | 0.1205 | 0.0278 | 0.0250 | 0.0072 | 2756 | 0.7686 | 0.1051 | 0.2366 | 0.044 | 0.0220 | 0.426 |

TABLE 14
Blockade of EGFR binding to EGF by pgEG-SX VHH antibodies

| | pgEG- | pgEG- | pgEG- | pgEG- | pgEG- | |
|---|---|---|---|---|---|---|
| nM | SX-3 | SX-6 | SX-22 | SX-40 | SX-57 | pgEG5 |
| 1000 | 0.336 | 0.646 | 0.960 | 0.151 | 0.150 | 1.219 |
| 200 | 0.382 | 0.668 | 0.903 | 0.147 | 0.158 | 1.204 |
| 40 | 0.482 | 0.606 | 0.801 | 0.414 | 0.433 | 1.109 |
| 8 | 0.562 | 0.757 | 0.736 | 0.633 | 0.649 | 1.066 |
| 1.6 | 0.723 | 0.928 | 0.864 | 0.922 | 0.919 | 1.181 |
| 0.32 | 0.806 | 1.075 | 0.955 | 1.028 | 1.084 | 1.155 |
| 0.064 | 0.979 | 0.956 | 0.983 | 1.065 | 1.135 | 1.151 |
| 0 | 1.057 | 1.039 | 1.071 | 1.143 | 1.107 | 1.195 |

### Example 5

### Anti-HSA VHH Antibodies

### Immunizations

Llamas were immunized at Abcore Inc following their standard protocols. Recombinant human HSA (SEQ ID NO:113) were mixed with Complete Freund's Adjuvant (day 0) or Incomplete Freund's Adjuvant (following immunizations). Six subcutaneous injections per llama was performed at 50 ug/dose at biweekly intervals At day 45, serum was collected from immunized llamas to define antibody titers by ELISA. In ELISA, 96-well Maxisorp plates were coated with 100 ng/well antigen. After blocking and adding diluted sera samples, the presence of specific antibodies was demonstrated using Horseradish Peroxidase (HRP) Conjugated Goat Anti-Llama IgG (H+L) antibody.

human serum albumin
SEQ ID NO: 113
MKWVTFISLLFLFSSAYSRGVFRRDAHKSEVAHRFKDLGEENFKALV
LIAFAQYLQQCPFEDHVKLVNEVTEFAKTCVADESAENCDKSLHTLF
GDKLCTVATLRETYGEMADCCAKQEPERNECFLQHKDDNPNLPRLVR
PEVDVMCTAFHDNEETFLKKYLYEIARRHPYFYAPELLFFAKRYKAA
FTECCQAADKAACLLPKLDELRDEGKASSAKQRLKCASLQKFGERAF
KAWAVARLSQRFPKAEFAEVSKLVTDLTKVHTECCHGDLLECADDRA
DLAKYICENQDSISSKLKECCEKPLLEKSHCIAEVENDEMPADLPSL
AADFVESKDVCKNYAEAKDVFLGMFLYEYARRHPDYSVVLLLRLAKT
YETTLEKCCAAADPHECYAKVFDEFKPLVEEPQNLIKQNCELFEQLG
EYKFQNALLVRYTKKVPQVSTPTLVEVSRNLGKVGSKCCKHPEAKRM
PCAEDYLSVVLNQLCVLHEKTPVSDRVTKCCTESLVNRRPCFSALEV
DETYVPKEFNAETFTFHADICTLSEKERQIKKQTALVELVKHKPKAT
KEQLKAVMDDFAAFVEKCCKADDKETCFAEEGKKLVAASQAALGL

### Phage Library Construction and Selections

Peripheral blood mononuclear cells were prepared from serum samples of days 45 for immunized llamas using Ficoll-Paque Plus (GE Healthcare) according to the manufacturer's instructions. Total RNA was extracted from the peripheral blood mononuclear cells using RNeasy Midi Kit (Qiagen) following manufacturer instructions and used as starting material for RT-PCR to amplify VHH encoding gene fragments. These fragments were cloned into a house made phagemid vector, allowing production of recombinant phage particles, after infection with helper phage, which display the VHH as gene-III fusion proteins on the surface of the phage particles. Phage was prepared according to standard methods and stored after filter sterilization at 4° C. for further use.

Phage libraries obtained from immunized llamas were used for selection. In the selection, biotinylated HSA was incubated with the phage libraries and subsequently captured on Streptavidin Dynabeads (Invitrogen). Following extensive washing, bound phages were eluted with either 1 mg/ml trypsin. The output from the selections was rescued in E. coli TG1 cells. Colonies were picked and sequenced at BATJ, Inc.

cDNAs encoded the positive VHH were synthesized with C-terminal His-tag at Atum (DNA2.0, Inc), and transiently transfected in HEK293 cells, and positive VHH were purified by IMAC chromatography for in vitro functional assay.

### Octet Kinetic Binding Analysis

Bio-Layer Interferometry (BLI), a label-free technology was used for measuring the binding kinetics of positive llama single domain antibodies. Affinity measurements were performed with Octet QKe equipped with Anti-Penta-His capture (HIS1K) biosensor tips (ForteBio®, Menlo Park, CA, USA). The assay was performed at 30° C. in 1×PBS buffer (Gibco®, PBS pH7.2). Samples were agitated at 1000 rpm. Prior to analysis, sensors were humidified for 15 minutes. Purified single domain antibodies was tested for its binding capacity with HIS1K sensor tips. Tips were loaded using 20 μg/ml of single domain antibodies. Loading proceeded for 300 s resulting in capture levels of between 1.8 and 2 nm. The test antigen were prepared for binding analysis by dilution to concentrations of 100, 150, 250, 350 nM in 1×PBS. Association was initiated and monitored for 200 s, after which tips were transferred to 1xPBS buffer without Factor protein (Gibco, PBS pH 7.2), in order to monitor dissociation. Sensor data was collected throughout the experiments, processed, and analyzed using the Octet data analysis software 7 (Forte Bio).

### Results

### Isolation of Anti-HSA Single Domain Antibodies

Positive phage colonies from immunized llama phage libraries were sequenced. Amino acid sequences are presented in Table 15. cDNA sequences based on amino acid sequences below were synthesized at Atum (DNA2.0) in pJ607 expression vector. Th expression plasmids was transfected into a HEK293 cell line to produce fully recombinant single domain antibodies with C-terminal his-tag. The expressed VHH were purified by HisTrap HP column.

TABLE 15
Anti-HSA Sequences

| | |
|---|---|
| MSA1 | EVQLVESGGGLAQAGGSLRLSCADSGHSFSLY |
| (SEQ ID | AMAWFRQTPEKEREFVAAISSSGGDTYYADSV |
| NO: 114) | KGRFTISRENAKNTAYLQMDSLKPGDTAVYFC |
| | AGNYYTSQLMRGMYEHWGQGTQVTVSS |
| MSA2 | EVQLVESGGGLAQAGGFLRLSCADSGHSFSLY |
| (SEQ ID | AMAWFRQTPEKEREFVAAISSSGGDTYYADSV |
| NO: 115) | KGRFTISRENAKNTVYLQMDSLKPGDTAVYFC |
| | AGNYYTSQLMRGMYEHWGQGTQVTVSS |
| MSA4 | EVQLVEFGGGLVQAGGSLRLSCAASGRTDSAY |
| (SEQ ID | RMGWFRQAPGKEREFVSAINWSDGRTVYLDSV |
| NO: 116) | KGRFTISRDNAKNMVYLQMNSLKPEDTAVYYC |
| | AADPDSRLYYTVPQNYDYWGQGTQVTVSS |
| MSA6 | QVQLVESGGGLVQPGGSLRLSCAASGLPSRVM |
| (SEQ ID | GWFRQAAGKEREFVASISVSGIDTRYGDSVKG |
| NO: 117) | RFTISRDNTKSSLYLQMNSLKPEDTAVYYCAA |
| | TDGQGNYRHWGQGTQVTVSS |
| MSA B1 | EVQLVESGGGLVQPGGSLHLSCAASRLTTGVT |
| (SEQ ID | FSDYGMAWFRQAPGAERELLANINWSGQYTNY |
| NO: 118) | RDSVKGRAAIFRDNAKNTLYLNINSLKAEDTA |
| | VYYCAASSRKYGPGIKSQYEWWGQGTQVTVSS |

Humanization of MSA4 Based on Human Germline IGHV3-23*04

Humanized MSA4-1
(SEQ ID NO: 119)
EVQLVESGGGLVQPGGSLRLSCAASGRTDSAYRMGWFRQAPGKERE
FVSAINWSDGRTVYLDSVKGRFTISRDNAKNTLYLQMNSLRAEDTA
VYYCAADPDSRLYYTVPQNYDYWGQGTQVTVSS

### ELISA Binding Assay of MSA VHHS

The 96-well plate was coated with 100 μL per well of HSA or MSA (murine serum albumin) prepared at 1 μg/ml in coating buffer at 4° C. overnight, then blocked with 200 μL per well of casein for one hour at room temperature. MSA VHHs were made into a series of dilutions and added to HSA or MSA coated plate for one hour incubation. The plate was washed with PBST four times, then incubated with Streptavidin conjugated with HRP at 25° C. for 1 hour. After three times of wash with 250 μL PBST per well, the plate was developed with 100 μL of TMB per well for 10 to 20 min in the dark and then stopped by adding 100 μL of Stop Solution. The plate was read at 450 nM Molecular Devices Microplate reader. Results are presented in Tables 16-19.

TABLE 16
ELISA binding results of MSA VHHs screening with HSA

| nM | MSA1 | | MSA2 | | MSA4 | | MSA6 | | MSAB1 | |
|---|---|---|---|---|---|---|---|---|---|---|
| 200 | 0.095 | 0.094 | 0.084 | 0.083 | 0.241 | 0.195 | 0.326 | 0.361 | 0.546 | 0.624 |
| 400 | 0.070 | 0.064 | 0.059 | 0.063 | 0.156 | 0.128 | 0.236 | 0.263 | 0.135 | 0.171 |
| 80 | 0.068 | 0.066 | 0.064 | 0.061 | 0.128 | 0.106 | 0.184 | 0.194 | 0.088 | 0.100 |
| 16 | 0.064 | 0.063 | 0.057 | 0.065 | 0.095 | 0.090 | 0.125 | 0.127 | 0.068 | 0.076 |
| 3.2 | 0.069 | 0.061 | 0.064 | 0.084 | 0.083 | 0.092 | 0.113 | 0.095 | 0.066 | 0.077 |
| 0.64 | 0.065 | 0.061 | 0.066 | 0.064 | 0.068 | 0.090 | 0.090 | 0.077 | 0.059 | 0.069 |
| 0.128 | 0.074 | 0.071 | 0.066 | 0.067 | 0.068 | 0.064 | 0.081 | 0.076 | 0.069 | 0.077 |
| 0.0256 | 0.075 | 0.083 | 0.069 | 0.070 | 0.071 | 0.086 | 0.064 | 0.067 | 0.063 | 0.080 |

TABLE 17
ELISA binding results of MSA VHHs screening with MSA

| nM | MSA1 | | MSA2 | | MSA4 | | MSA6 | | MSAB1 | |
|---|---|---|---|---|---|---|---|---|---|---|
| 200 | 0.131 | 0.184 | 0.125 | 0.160 | 0.620 | 0.534 | 0.100 | 0.108 | 0.486 | 0.585 |
| 400 | 0.064 | 0.062 | 0.068 | 0.067 | 0.471 | 0.388 | 0.073 | 0.078 | 0.162 | 0.221 |
| 80 | 0.062 | 0.060 | 0.062 | 0.061 | 0.323 | 0.283 | 0.067 | 0.067 | 0.096 | 0.102 |
| 16 | 0.059 | 0.061 | 0.059 | 0.057 | 0.217 | 0.215 | 0.057 | 0.061 | 0.062 | 0.066 |
| 3.2 | 0.063 | 0.062 | 0.06 | 0.061 | 0.133 | 0.139 | 0.080 | 0.067 | 0.071 | 0.078 |
| 0.64 | 0.064 | 0.061 | 0.059 | 0.058 | 0.082 | 0.080 | 0.055 | 0.056 | 0.061 | 0.063 |
| 0.128 | 0.063 | 0.066 | 0.066 | 0.067 | 0.070 | 0.070 | 0.087 | 0.065 | 0.066 | 0.081 |
| 0.0256 | 0.066 | 0.071 | 0.061 | 0.059 | 0.065 | 0.06 | 0.070 | 0.060 | 0.059 | 0.069 |

TABLE 18
ELISA result of humanized MSA4-1 binding to HSA

| nM | MSA4 | MSA4-1 |
|---|---|---|
| 1000 | 0.655 | 0.818 |
| 200 | 0.417 | 0.363 |
| 40 | 0.250 | 0.267 |
| 8 | 0.135 | 0.156 |
| 1.6 | 0.024 | 0.053 |
| 0.32 | 0.009 | 0.003 |
| 0.064 | 0.028 | 0.009 |
| 0.0128 | 0.073 | 0.064 |

TABLE 19
ELISA result of humanized MSA4-1 binding to MSA

| nM | MSA4 | MSA4-1 |
|---|---|---|
| 1000 | 1.934 | 1.798 |
| 200 | 1.668 | 1.274 |
| 40 | 1.257 | 0.774 |
| 8 | 0.673 | 0.347 |
| 1.6 | 0.149 | 0.100 |
| 0.32 | 0.083 | 0.059 |
| 0.064 | 0.051 | 0.029 |
| 0.0128 | 0.001 | 0.002 |

### BLI Binding Kinetic Analysis of MSA VHHs

Kinetic binding analysis of MSA VHHs were performed with Gator Prime system equipped with analyze software. The Gator assay protocol was employed for all the experiments in this report described below. The experiments were done at 30° C. Prior to analysis, sensors were socked in Q buffer for 600 sec with shaking at 1000 rpm, and then 2 μM of HIS tagged MSA VHHs were loaded to Anti-His sensors with shaking at 400 rpm for 120 sec. Loaded sensors were transferred into Q buffer for 60 sec. Association of HSA or MSA at 100 nM was initiated and monitored for 120-180 sec with shaking at 1000 rpm, and finally dissociation in Q buffer for 120-180 sec with 1000 rpm shaking. Software V2.0 (Gator Bio) was used for data analysis. The on-rate (Kₒₙ) and off-rate (K_{d}) were determined by fitting of the association and dissociation phases of each sample. The mathematical model used assumes a 1:1 stoichiometry, fitting only one analyte in solution binding to one binding site on the surface. The equilibrium dissociation constant (K_{D}), a measure for affinity, was then calculated as the ratio of K_{d} and Kₒₙ. Results are presented in Tables 20-23.

TABLE 20
Binding Affinity of MSA VHHs with HSA

| | k_{off} (1/s) | kₒₙ (1/Ms) | K_{D} (M) |
|---|---|---|---|
| MSA-1 | 2.17E−02 | NA | NA |
| MSA-2 | 2.25E−02 | NA | NA |
| MSA-4 | 1.01E−03 | 8.96E+04 | 1.13E−08 |
| MSA-6 | 1.25E−03 | 5.98E+04 | 2.09E−08 |
| MSA-B1 | 5.11E−03 | 4.54E+05 | 1.13E−08 |

TABLE 21
Binding Affinity of MSA VHHs with MSA

| | K_{off} (1/s) | Kₒₙ (1/Ms) | K_{D} (M) |
|---|---|---|---|
| MSA-1 | 1.17E−03 | 1.76E+05 | 6.64E−09 |
| MSA-2 | 1.26E−03 | 1.75E+05 | 7.18E−09 |
| MSA-4 | 6.44E−04 | 1.43E+05 | 4.49E−09 |
| MSA-6 | 2.44E−03 | 4.44E+04 | 5.49E−08 |
| MSA-B1 | 6.05E−04 | 1.14E+05 | 5.32E−09 |

TABLE 22
Binding Affinity of humanized MSA-4 with HSA

| | Antigen | koff(1/s) | kon(1/Ms) | KD(M) |
|---|---|---|---|---|
| MSA-4 | HSA | 7.52E−04 | 1.05E+05 | 7.16E−09 |
| hMSA4-1 | HSA | 5.45E−04 | 1.01E+05 | 5.38E−09 |

TABLE 23
Binding Affinity of humanized MSA-4 with MSA

| | Antigen | koff(1/s) | kon(1/Ms) | KD(M) |
|---|---|---|---|---|
| MSA-4 | MSA | 1.10E−03 | 1.25E+05 | 8.83E−09 |
| hMSA4-1 | MSA | 6.78E−04 | 1.21E+05 | 5.58E−09 |

### Example 6

### Anti-IL22 VHH Antibodies

### Immunizations

Llamas were immunized at Abcore Inc following their standard protocols. Recombinant human IL-22 (SEQ ID NO:120) were mixed with Complete Freund's Adjuvant (day 0) or Incomplete Freund's Adjuvant (following immunizations). Six subcutaneous injections per llama was performed at 50 μg/dose at biweekly intervals At day 45, serum was collected from immunized llamas to define antibody titers by ELISA. In ELISA, 96-well Maxisorp plates were coated with 100 ng/well antigen. After blocking and adding diluted sera samples, the presence of specific antibodies was demonstrated using Horseradish Peroxidase (HRP) Conjugated Goat Anti-Llama IgG (H+L) antibody.

human IL-22
SEQ ID NO: 120
MAALQKSVSSFLMGTLATSCLLLLALLVQGGAAAPISSHCRLDKSN
FQQPYITNRTFMLAKEASLADNNTDVRLIGEKLFHGVSMSERCYLM
KQVLNFTLEEVLFPQSDRFQPYMQEVVPFLARLSNRLSTCHIEGDD
LHIQRNVQKLKDTVKKLGESGEIKAIGELDLLFMSLRNACI

### Phage Library Construction and Selections

Peripheral blood mononuclear cells were prepared from serum samples of days 45 for immunized llamas using Ficoll-Paque Plus according to the manufacturer's instructions. Total RNA was extracted from the peripheral blood mononuclear cells using RNeasy Midi Kit following manufacturer instructions and used as starting material for RT-PCR to amplify VHH encoding gene fragments. These fragments were cloned into a house made phagemid vector, allowing production of recombinant phage particles, after infection with helper phage, which display the VHH as gene-Ill fusion proteins on the surface of the phage particles. Phage was prepared according to standard methods and stored after filter sterilization at 4° C. for further use.

Phage libraries obtained from immunized llamas were used for selection. In the selection, biotinylated HSA was incubated with the phage libraries and subsequently captured on Streptavidin Dynabeads. Following extensive washing, bound phages were eluted with either 1 mg/ml trypsin. The output from the selections was rescued in E. coli TG1 cells. Colonies were picked and sequenced at BATJ, Inc.

CDNAs encoded the positive VHH were synthesized with C-terminal His-tag at Atum (DNA2.0, Inc), and transiently transfected in HEK293 cells, and positive VHH were purified by IMAC chromatography for in vitro functional assay.

### Octet Kinetic Binding Analysis

Bio-Layer Interferometry (BLI), a label-free technology was used for measuring the binding kinetics of positive llama single domain antibodies. Affinity measurements were performed with Octet QKe equipped with Anti-Penta-His capture (HIS1K) biosensor tips (ForteBio®). The assay was performed at 30 C in 1×PBS buffer (Gibco®, PBS pH7.2). Samples were agitated at 1000 rpm. Prior to analysis, sensors were humidified for 15 minutes. Purified single domain antibodies was tested for its binding capacity with HIS1K sensor tips. Tips were loaded using 20 μg/ml of single domain antibodies. Loading proceeded for 300 s resulting in capture levels of between 1.8 and 2 nm. The IL-22 antigen were prepared for binding analysis by dilution to concentrations of 100, 150, 250, 350 nM in 1×PBS. Association was initiated and monitored for 200 s, after which tips were transferred to 1×PBS buffer without Factor protein (Gibco, PBS pH 7.2), in order to monitor dissociation. Sensor data was collected throughout the experiments, processed, and analyzed using the Octet data analysis software 7 (Forte Bio).

### Results

### Isolation of Anti-IL22 Single Domain Antibodies

Positive phage colonies from immunized llama phage libraries were sequenced. Amino acid sequences are presented in Table 24. cDNA sequences based on amino acid sequences below were synthesized at Atum (DNA2.0) in pJ607 expression vector. Th expression plasmids was transfected into a HEK293 cell line to produce fully recombinant single domain antibodies with C-terminal his-tag. The expressed VHH were purified by HisTrap HP column.

TABLE 24
Llama anti-IL22 VHH Sequences

| | |
|---|---|
| IL22-A3 | QVQLVESGGGLVQPGGSLRLSCEASGRTFSSVSMGWFRQAPGKERVIV |
| (SEQ ID NO: 121) | AAADWSGTTYYTGSLKGRFTISRDNAKNMVYLQMNSLKPEDTAVYYCA |
| | ASDPRRSAYKYWGQGTQVTVSS |
| IL22-223 | QVQLVESGGGLAQPGGSLRLSCAVSGFTLNYYVIAWFRQAPGKEREGV |
| (SEQ ID NO: 122) | SCISSSDGSTYYTDSVKGRFTISRDNAKNTVYLQMDSLKPEDTAVYYC |
| | AADLRAYYTVPLWASEYDYWGQGTQVTVSS |
| IL22-354 | EVQLVESGGGLVQPGGSLRLSCAASGFTLDYYAIAWFRQAPGKEREGV |
| (SEQ ID NO: 123) | SCISTSDGSTYYADSVKGRFTISRDNAKNTVYLQMNSLKPEDTAVYYC |
| | AADPSPFFVAPLVDYEYDYWGQGTQVTVSS |
| Humanized IL22-223, | EVQLVESGGGLVQPGGSLRLSCAASGFTLNYYVMSWFRQAPGKEREGV |
| Humanization of IL22- | SAISSSDGSTYYADSVKGRFTISRDNSKNTLYLQMNSLRAEDTAVYYC |
| 223 based on human | AADLRAYYTVPLWASEYDYWGQGTVSS |
| Germline IGHV3-23*04 | |
| (SEQ ID NO: 124) | |

Results of an Octet binding affinity assay of IL22 VHHs are presented in Table 25.

TABLE 25
Binding Affinity (KD) of IL22 VHHs with rh-IL22

| | koff(1/s) | kon(1/Ms) | KD(M) |
|---|---|---|---|
| Il22-A3 | 4.05E−04 | 4.96E+05 | 8.17E−10 |
| IL22-223 | 3.60E−04 | 2.78E+05 | 1.29E−09 |
| IL22-354 | 1.84E−04 | 5.03E+05 | 3.66E−10 |
| Humanized | 5.47E−04 | 5.09E+05 | 1.07E−09 |
| IL22-223 | | | |

### Example 7

### Multispecific Single Chain Antibodies

To construct a multispecific single chain antibodies, one or more of, anti-OX40, anti-CD40, anti-4-1BB, anti-EGFR, anti-IL22, anti-HSA, anti-CD47, anti-CD16, anti-PD-L1, anti-CD33, and anti-LAG3 VHH sequences are fused together via linkers in different configurations by recombinant DNA technology. WO2021/062361A2 is incorporated by reference herein for all it discloses regarding VHH sequences specific for HSA, PD-L1, CD33, CD16, and LAG3.

Exemplary non-cleavable and cleavable linker sequences are presented in Table 26. These constitute linker means or means for linking protein domains. These mean can be further characterized as cleavable or non-cleavable.

TABLE 26
Non-cleavable and cleavable linker sequences

| Linker Reference | Sequence | Cleavable? |
|---|---|---|
| L1 | GGGGSGGGS | No |
| (SEQ ID NO: 84) | | |
| L2 | (G₄S)n(G₃S)m | No |
| (SEQ ID NO: 85) | (n = 1-35; | |
| | m = 0-35) | |
| L3 | AAA | No |
| (SEQ ID NO: 86) | | |
| L4 | (G₄S)n | No |
| (SEQ ID NO: 87) | | |
| L11*3 | GGRGPLGLAGS | Yes |
| (SEQ ID NO: 88) | RSAFGGS | |
| L11*4 | GSPLGLAGS | Yes |
| (SEQ ID NO: 89) | | |
| L11*5 | GGSGPLGLAGS | Yes |
| (SEQ ID NO: 90) | RSAFG | |
| L11*6 | GPLGLAGSRSA | Yes |
| (SEQ ID NO: 91) | GGSQVQL | |
| L11*7 | GSGPLGLAARS | Yes |
| (SEQ ID NO: 92) | AGGS | |
| L11*8 | GSGPLGLAARS | Yes |
| (SEQ ID NO: 93) | AFGGS | |
| L11*9 | GGSGRSAPLGL | Yes |
| (SEQ ID NO: 94) | ARQARQVGGS | |
| L11*10 | GGSGRSAPLGL | Yes |
| (SEQ ID NO: 95) | GRQARGGS | |
| L11*11 | GGSPLGLARQA | Yes |
| (SEQ ID NO: 96) | RGSGRSAGGS | |
| L11*12 | GGSGRSAPLGL | Yes |
| (SEQ ID NO: 97) | ARQARVVGGS | |
| L11*13 | GSRQARVVGS | Yes |
| (SEQ ID NO: 98) | | |
| L11*14 | GSRQRRVVGS | Yes |
| (SEQ ID NO: 99) | | |
| L11*15 | GSRQARGS | Yes |
| (SEQ ID NO: 100) | | |
| L11*16 | GSRQRRGS | Yes |
| (SEQ ID NO: 101) | | |
| L11*17 | GSRQARGGS | Yes |
| (SEQ ID NO: 102) | | |
| L11*18 | GSRQRRGGS | Yes |
| (SEQ ID NO: 103) | | |

Sequences of multi-specific antibodies are depicted in Tables 27 and 28.

TABLE 27
Bi-specific antibodies

| | |
|---|---|
| SM2230-103 | EVQLVESGGGLVQPGGSLRLSCAASGGGRTFSNYALGWFRQAPGKER |
| pgEG-SX-40-403- | EFVAAISRSGGNINYADSVKGRFTISRDNFKNTLYLQMSSLRPEDTA |
| F3Y-Fc(S)- | VYYCAAHYLLLPSYISTSTNMYNYWGQGTQVTVKPGGGGDKTHTCPP |
| pgDD40-SZ-3-4F1 | CPAPELLGGPSVFLFPPKPKDTLMISRTPEVTCVVVDVSHEDPEVKF |
| Specific for | NWYVDGVEVHNAKTKPREEQYASTYRVVSVLTVLHQDWLNGKEYKCK |
| EGFR and CD47 | VSNKALPAPIEKTISKAKGQPREPQVYTLPPSREEMTKNQVSLTCLV |
| (SEQ ID NO: 107) | KGFYSSDIAVEWESNGQPENNYKTTPPVLDSDGSFFLYSKLTVDKSR |
| | WQQGNVFSCSVMHEALHNHYTQKSLSLSPGSGGGGSGGGGSQVQLVE |
| | SGGGLVQPGGSLRLSCSASGRAFNTYTMAWFRQAPGKERDFVAAISR |
| | DGTITYYADSVKGRFTISRDNSKNTLYLQMSSLRAEDTAVYYCAARR |
| | VGAVPERESAYEHWGQGTQVTVSS* |
| SM2230-106 | QVQLVESGGGLVQPGGSLRLSCSASGRAFNTYTMAWFRQAPGKERDF |
| pgEG-SX-40-403- | VAAISRDGTITYYADSVKGRFTISRDNSKNTLYLQMSSLRAEDTAVY |
| F3Y-Fc(S)- | YCAARRVGAVPERESAYEHWGQGTQVTVKPGGGGDKTHTCPPCPAPE |
| pgDD40-SZ-3-4F1 | LLGGPSVFLFPPKPKDTLMISRTPEVTCVVVDVSHEDPEVKFNWYVD |
| CD47-Fc-CD40 | GVEVHNAKTKPREEQYASTYRVVSVLTVLHQDWLNGKEYKCKVSNKA |
| (SEQ ID NO: 108) | LPAPIEKTISKAKGQPREPQVYTLPPSREEMTKNQVSLTCLVKGFYS |
| | SDIAVEWESNGQPENNYKTTPPVLDSDGSFFLYSKLTVDKSRWQQGN |
| | VFSCSVMHEALHNHYTQKSLSLSPGSGGGGSGGGGSEVQLVESGGGL |
| | VQPGGSLRLSCAASGGGRTFSNYALGWFRQAPGKEREFVAAISRSGG |
| | NINYADSVKGRFTISRDNFKNTLYLQMSSLRPEDTAVYYCAAHYLLL |
| | PSYISTSTNMYNYWGQGTQVTVSS* |
| SM2248-177 | EVQLVESGGGLVQPGGSLRLSCAASGGGRTFSNYALGWFRQAPGKER |
| hA09-10*-Fc(S)- | EFVAAISRSGGNINYADSVKGRFTISRDNFKNTLYLQMSSLRPEDTA |
| pgDD40- | VYYCAAHYLLLPSYISTSTNMYNYWGQGTQVTVKPGGGGDKTHTCPP |
| SZ-3-4F1 | CPAPELLGGPSVFLFPPKPKDTLMISRTPEVTCVVVDVSHEDPEVKF |
| Specific for | NWYVDGVEVHNAKTKPREEQYASTYRVVSVLTVLHQDWLNGKEYKCK |
| CD40 and CD47 | VSNKALPAPIEKTISKAKGQPREPQVYTLPPSREEMTKNQVSLTCLV |
| (SEQ ID NO: 109) | KGFYSSDIAVEWESNGQPENNYKTTPPVLDSDGSFFLYSKLTVDKSR |
| | WQQGNVFSCSVMHEALHNHYTQKSLSLSPGSGGGGSGGGGSQVQLVE |
| | SGGGLVQPGGSLRLSCSASGRAFNTYTMAWFRQAPGKERDFVAAISR |
| | DGTITYYADSVKGRFTISRDNSKNTLYLQMSSLRAEDTAVYYCAARR |
| | VGAVPERESAYEHWGQGTQVTVSS* |
| SMSM2235-113 | QVQLVESGGGLVQPGGSLRLSCTASGIRFSFYTLGWYRQAPGKGREL |
| specific for | VAEIYSSDNAKYADSVKGRFTISRDNAKNSLYLQMNSLRAEDTAVYY |
| EGFR and CD16A | CNVRATDYWGPGTQVTVKPGGGGDKTHTCPPCPAPELLGGPSVFLFP |
| (SEQ ID NO: 112) | PKPKDTLMISRTPEVTCVVVDVSHEDPEVKFNWYVDGVEVHNAKTKP |
| | REEQYASTYRVVSVLTVLHQDWLNGKEYKCKVSNKALPAPIEKTISK |
| | AKGQPREPQVYTLPPSREEMTKNQVSLTCLVKGFYSSDIAVEWESNG |
| | QPENNYKTTPPVLDSDGSFFLYSKLTVDKSRWQQGNVFSCSVMHEAL |
| | HNHYTQKSLSLSPGGGGSGGGGSGGGGSQVQLVESGGGLVQPGGSLR |
| | LSCRASGFTFSNHAMSWVRQAPGKGLEWVSEISFNGHATRYADSVKG |
| | RFTISRDNSKNTLYLQMNSLRAEDTAVYYCRKGWNATPQIGERGRGT |
| | QVTVSS* |

*Stop codon

TABLE 28
Multi-specific antibodies

| | |
|---|---|
| 2270, pgEG-SX-40-403-F3Y- | QVQLVESGGGLVQPGGSLRLSCTASGIRFSFYTLGWYRQAPGKGREL |
| hPL14*--pgDD40-SZ-3- | VAEIYSSDNAKYADSVKGRFTISRDNAKNSLYLQMNSLRAEDTAVYY |
| 4F1-GS15-MSA4-1 | CNVRATDYWGPGTQVTVSSGGGGSGGGSEVQLVESGGGLVQPGGSLR |
| EGFR-PD-L1-CD40-HSA | LSCAASGSTSGIYDMGWYRQAPGKLREVVSVITSGGTTYYADSVKGR |
| (SEQ ID NO: 110) | FTISRDNSKNTLYLQMNSLRAEDTAVYYCNIRTRLIIWGQGTQVTVS |
| | SGGGGSGGGSQVQLVESGGGLVQPGGSLRLSCSASGRAFNTYTMAWF |
| | RQAPGKERDFVAAISRDGTITYYADSVKGRFTISRDNSKNTLYLQMS |
| | SLRAEDTAVYYCAARRVGAVPERESAYEHWGQGTQVTVSSGGGGSGG |
| | GGSGGGGSEVQLVESGGGLVQPGGSLRLSCAASGRTDSAYRMGWFRQ |
| | APGKEREFVSAINWSDGRTVYLDSVKGRFTISRDNAKNTLYLQMNSL |
| | RAEDTAVYYCAADPDSRLYYTVPQNYDYWGQGTQVTVSS |
| 2270-0231, pgEG-SX-40- | QVQLVESGGGLVQPGGSLRLSCTASGIRFSFYTLGWYRQAPGKGREL |
| 403-F3Y-hPL14*-MSA4-1-- | VAEIYSSDNAKYADSVKGRFTISRDNAKNSLYLQMNSLRAEDTAVYY |
| pgDD40-SZ-3-4F1 | CNVRATDYWGPGTQVTVSSGGGGSGGGSEVQLVESGGGLVQPGGSLR |
| EGFR-PD-L1-HSA-CD40 | LSCAASGSTSGIYDMGWYRQAPGKLREVVSVITSGGTTYYADSVKGR |
| (SEQ ID NO: 111) | FTISRDNSKNTLYLQMNSLRAEDTAVYYCNIRTRLIIWGQGTQVTVS |
| | SGGGGSGGGSEVQLVESGGGLVQPGGSLRLSCAASGRTDSAYRMGWF |
| | RQAPGKEREFVSAINWSDGRTVYLDSVKGRFTISRDNAKNTLYLQMN |
| | SLRAEDTAVYYCAADPDSRLYYTVPQNYDYWGQGTQVTVSSGGGGSG |
| | GGSQVQLVESGGGLVQPGGSLRLSCSASGRAFNTYTMAWFRQAPGKE |
| | RDFVAAISRDGTITYYADSVKGRFTISRDNSKNTLYLQMSSLRAEDT |
| | AVYYCAARRVGAVPERESAYEHWGQGTQVTVSS |

Also disclosed herein are mutations of proline (P) to serine(S) at certain positions to reduce dimer and aggregate formation.

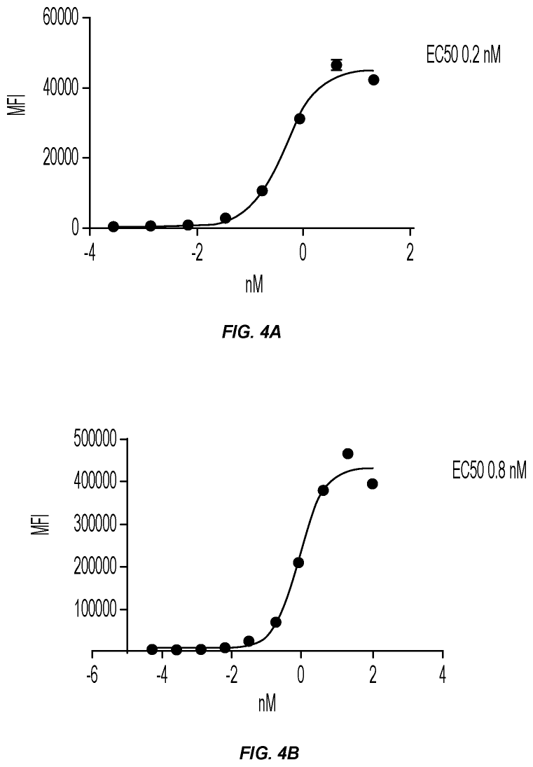

Results of an ELISA assay of binding of SM2248 to rh-CD47 and rh-CD40 is presented in FIG. 3A-B.

### Flow Cytometry Assay of SM2248

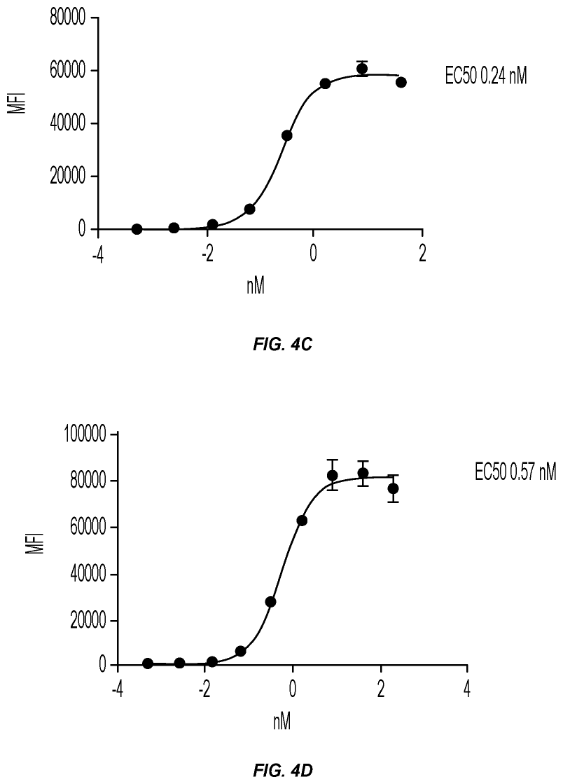

Cells (viabilities >95%) were washed by ice cold PBS and resuspended in staining buffer (PBS-2% FBS) at the cell density 1×10⁶/mL. The resuspended cells were applied into 96-well plates with 90 μL per well. A series of dilution of tested compounds prepared in the staining buffer were added to the 96 well plate with 10 μL each well and incubated for 45 min at room temperature. The 96-well plates were washed 2 to 3 times with staining buffer, and then incubated with secondary conjugated detection antibody for 25 min in the dark. Data were acquired by Flow cytometer and analyzed by Software Flowjo. Human CD47 overexpressing CHO (CHO-hCD47), human CD40 overexpressing CHO (CHO-hCD40), and two tumor cell lines, Raji and A431, were used for the assay. The assay shown SM2248 binding to CD47 or CD40 on the cell surface with subnanomolar affinity (FIGS. 4A-D).

### CD40 Reporter Assay of SM2248

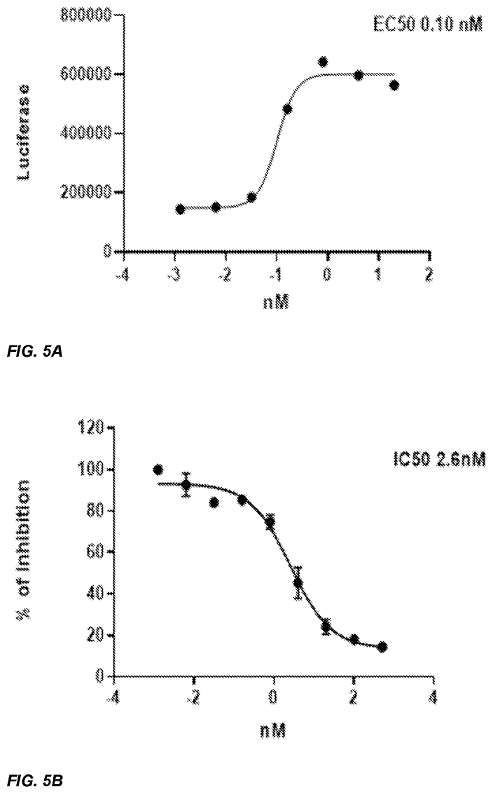

CHO-CD47 cells in logarithmic growth phase (viability greater than 95%) were digested with 0.25% trypsin and treated with RPMI1640 complete medium immediately. After centrifuged at 150×g for 5 min, cell pellets were resuspended in RPMI1640 complete medium at the density of 2×10⁶ cells/ml and added to the 96 well plate with 40 μL per well. The 96-well plate was then incubated overnight in 37° C. CO₂ incubator. A series of dilution of SM2248 in the DMEM medium were added to the 96 well plate with 10 μL each well. The 96-well plate was then incubated for 60 min in 37° C. CO₂ incubator. 293T-NFKB-CD40 reporter cells in logarithmic growth phase (viability greater than 95%) were digested with 0.25% trypsin and treated with RPMI 1640 complete medium immediately. After centrifuged at 150×g for 5 min, cell pellets were resuspended in RPMI1640 complete medium at the density of 4×10⁵ cells/ml and added to the 96 well SM2248 treated plate with 50 μL per well. The 96-well plate was then incubated for 6 hours in 37° C.-CO₂ incubator. After incubation, the cells from each well were transferred into a 96-well black plate. Substrate from One-Lite Luciferase Assay System was prepared following the manufacturer's instructions. Aliquots of 100 μL substrate were added to each well. The assay plate was measured in GLOMAX 96 microplate luminometer using program “CellTiterGlo”. SM2248 is a CD40 agonist bispecific antibody and activated CD40 in a dose-dependent manner (FIG. 5A).

### Flow Cytometry Blocking Assay of SM2248

Jurkat cells in logarithmic growth phase (viability greater than 95%), washed twice with ice cold PBS, were resuspended in the staining buffer (PBS+2% FBS) at the cell density 1×10⁶/mL and then applied into 96-well plate with 90 μL per well. A series of dilution of SM2248 in the staining buffer and SIRPa-his were added to Jurkat cells with 10 μL each well. The final concentration of SIRPa-his in each well is 1 μg/mL. The 96-well plate was then incubated for 30 min at room temperature. After incubation the assay plate was washed with 250 μL of the staining buffer three times by certification at 500×g for 3 min. The cell pellets in each well were resuspended in 100 μL of 1:100 diluted PE anti-His detection antibody and kept in the dark for 25 min at room temperature. The 96-well plate was washed with staining buffer three times and then resuspended in 150 μL cold PBS buffer. The plate was read in Agilent, NovoCyte and data was analyzed by Flowjo software. The assay indicated that SM2248 potently blocked SIRP binding to CD47 on Jurkat cell surface with IC_{50 2.6} nM (FIG. 5B).

These results indicate that SM2248 is a CD47 target dependent agonist and activated CD40 in a dose dependent manner.

The BLI Binding affinity (KD) assay of SM2248 is summarized in Table 29. SM2248 bound to human and cynomolgus-monkey (Cyno) CD40 and CD47 with similar binding affinity but less binding affinity to mouse and rat proteins.

TABLE 29
BLI Binding Affinity (KD) Assay of SM2248

| Antigen | Koff (1/s) | Kon (1/Ms) | KD (M) |
|---|---|---|---|
| rh-CD40 | 5.86E−05 | 8.28E+05 | 7.07E−11 |
| Cyno-CD40* | 3.36E−05 | 6.36E+05 | 5.28E−11 |
| Rat-CD40 | /¹ | / | / |
| Mouse-CD40 | / | / | / |
| Rh-CD47 | 7.11E−05 | 8.88E+05 | 8.00E−11 |
| Cyno-CD47 | 1.48E−03 | 1.95E+06 | 7.57E−10 |
| Rat-CD47 | / | / | / |
| Mouse-CD47 | / | / | / |

¹No binding was observed.

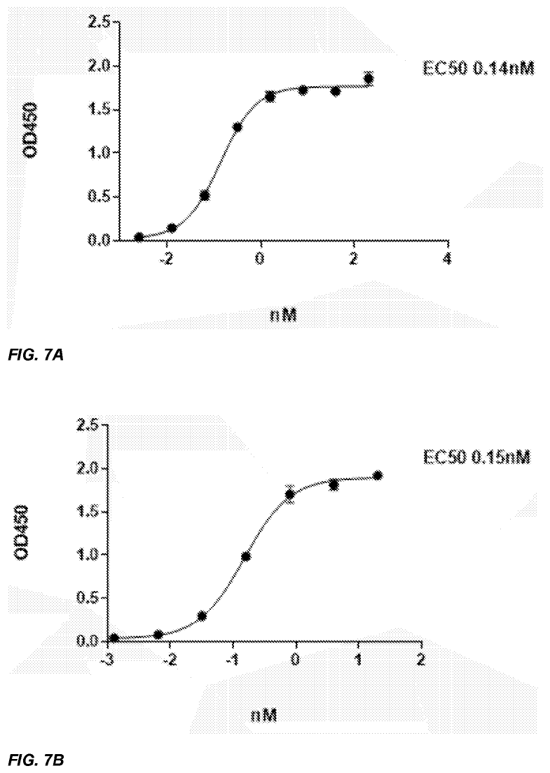

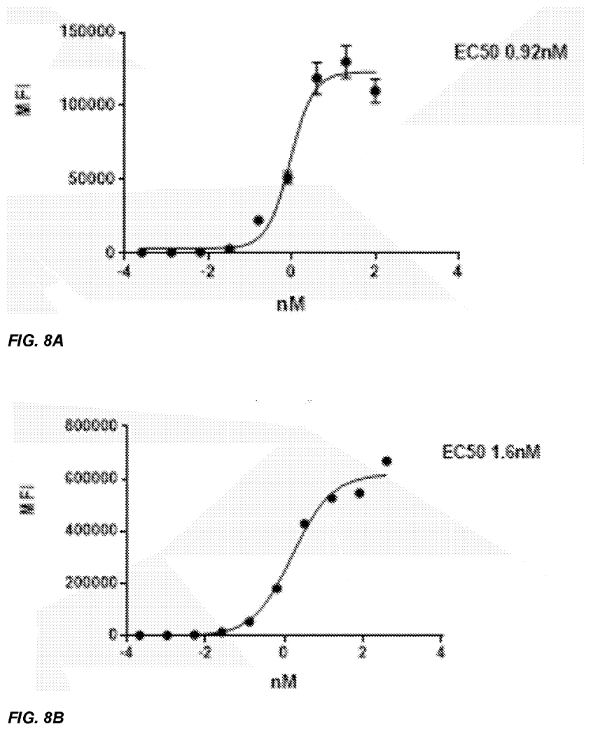

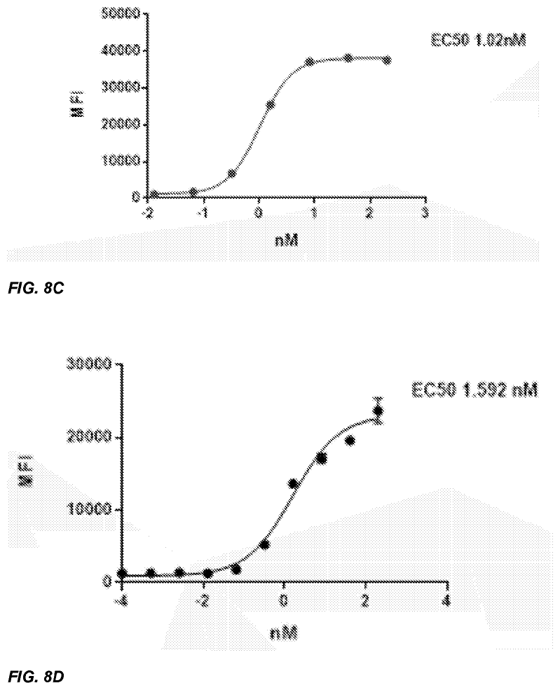

Results of an ELISA binding assay for SM2235 against recombinant human EGFR (rh-EGFR) and rh-CD16A are presented in FIG. 7 and indicate that SM2235 bound to recombinant human EGFR and human CD16A with subnanomolar affinity. Flow cytometry analysis of SM2235 binding is presented in FIGS. 8 and 9.

Results of a BLI binding assay of SM2235 is presented in Table 30. The BLI binding assay indicated that SM2235 bound to human and cyno-monkey EGFR or CD16A with similar KD, but not binding to rat and mouse. SM2235 does not cross-react with rh-ErbB2,rh-ErbB3 and rh-ErbB4.

TABLE 30
BLI binding affinity assay of SM2235

| Antigen | Koff (1/s) | Kon (1/Ms) | KD (M) |
|---|---|---|---|
| rh-EGFR | 1.28E−04 | 3.69E+05 | 3.46E−10 |
| Cyno-EGFR | 1.70E−04 | 8.50E+05 | 2.00E−10 |
| rh-ErbB2 | / | / | / |
| rh-ErbB3 | / | / | / |
| rh-ErbB4 | / | / | / |
| Mouse-EGFR | / | / | / |
| Rat-EGFR | / | / | / |
| rh-CD16A | 4.28E−05 | 1.11E+06 | 3.85E−11 |
| Cyno-CD16A | 4.28E−05 | 1.11E+06 | 3.86E−10 |
| Rat-CD16 | / | / | / |
| Mouse-CD16 | / | / | / |

### Blocking Assay

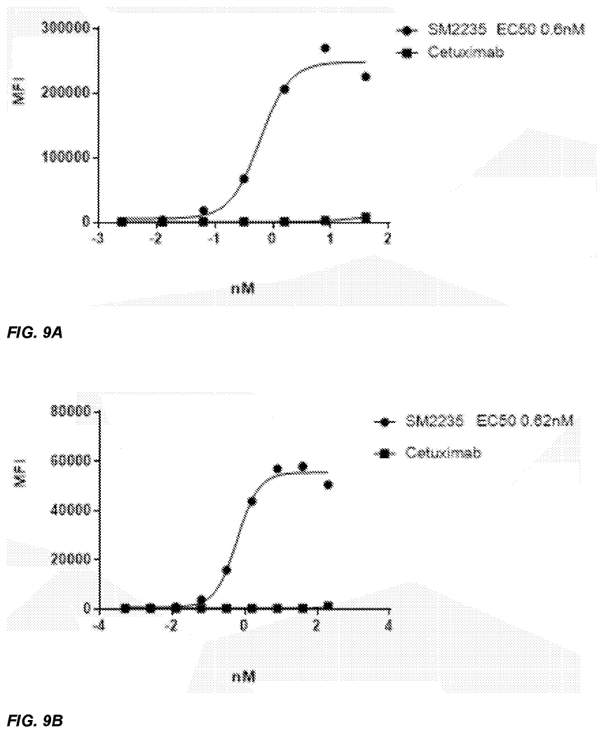

CHO-EGFR in logarithmic growth phase (viability greater than 95%), washed twice with ice cold PBS, were resuspended in the staining buffer (PBS+2% FBS) at the cell density 1x10⁶/mL and then applied into 96-well plate with 90 μL per well. A series of dilution of SM2235 in the staining buffer and EGF-his were added to Jurkat cells with 10 μL each well. The final concentration of EGF-his in each well is 1 μg/mL. The 96-well plate was then incubated for 30 min at room temperature. After incubation the assay plate was washed with 250 μL of the staining buffer three times by certification at 500×g for 3 min. The cell pellets in each well were resuspended in 100 μL of 1:100 diluted PE anti-His detection antibody and kept in the dark for 25 min at room temperature. The 96-well plate was washed with staining buffer three times and then resuspended in 150 μL cold PBS buffer. The plate was read in Agilent, NovoCyte and data was analyzed by Flowjo software. Results are presented in FIG. 10.

### NK Cytotoxicity Assay

### Method

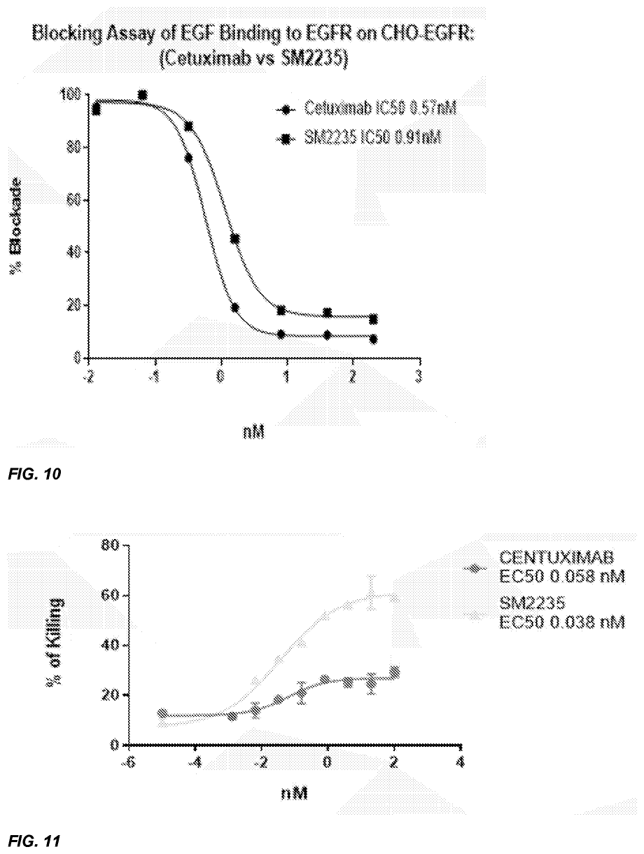

A431 cells were resuspended in complete medium KBM581 at the cell density of 1.25×10⁵/mL. Expanded human NK (eNK) cells were thawed and resuspended in complete medium KBM581 at 1×106 cells/mL. A431 (40 μL) and eNK cells (50 μL) were applied into each well of 96-well plate. The ratio of effector cells (eNK) to target cells (A431) was 10:1. SM2235 stocks were prepared as 5-fold dilution series in complete medium KBM581 from the top concentration of 1000 nM were added into cell plate with 10 μL per well. The concentrations of SM2235 in the wells were 100 nM, 20 nM, 40 nM, 8 nM, 1.6 nM, 0.32 nM, 0.064 nM, 0.0128 nM, and 0 nM. The control groups were prepared as: eNK cell alone, A431 alone, A431 lysis and complete medium KBM581. The measurement of specific cell death was done according to the instruction of CytoTox-Glo™ Cytotoxicity Assay Kit. Briefly, after incubation at 37° C.-5% CO₂ for 4 hours, the plate was gently mixed and samples from each well were transferred into a black 96-well flat bottom plate. AAF-Glo reagent was prepared according to the instruction and added to each well with 50 μL. A431 lysis group was treated with 50 μL of the lysis reagent. The plate was incubated at room temperature in the dark for 15 minutes. The built-in program “CellTiterGlo” in Promega microplate luminescence detector was used to measure the luciferase activity of each well. Results are presented in FIG. 11.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A variable heavy (VHH) domain having an antigen-binding specificity for OX40, wherein the VHH domain has the amino acid sequence of one of SEQ ID NOs: 2-24, 106, or 125-142.

2. A VHH domain having an antigen-binding specificity for CD40, wherein the VHH domain has the amino acid sequence of one of SEQ ID NOs: 26-40.

3. A VHH domain having an antigen-binding specificity for 4-1BB, wherein the VHH domain has the amino acid sequence of one of SEQ ID NOs: 42-53.

4. A VHH domain having an antigen-binding specificity for the epidermal growth factor receptor (EGFR), wherein the VHH domain has the amino acid sequence of one of SEQ ID NOs: 55-83.

5. A VHH domain having an antigen-binding specificity for human serum albumin (HSA), wherein the VHH domain has the amino acid sequence of one of SEQ ID NOs: 114-118.

6. A VHH domain having an antigen-binding specificity for IL-22, wherein the VHH domain has the amino acid sequence of one of SEQ ID NOs: 121-124.

7. An antibody comprising a VHH domain of any one of claims 1-6.

8. A multi-specific antibody comprising a antibody binding domain with first binding specificity and a second antibody binding domain with a second binding specificity that is different than the first binding specificity wherein the first, binding specificity is specific for OX40, CD40, 4-1BB, HSA, IL-22, or EGFR; wherein
(a) the OX40 binding specificity is represented by the amino acid sequence of one of SEQ ID NOs: 2-24, 106, or 125-142;
(b) the CD40 binding specificity is represented by the amino acid sequence of one of SEQ ID NOs: 26-40;
(c) the 4-1BB binding specificity is represented by the amino acid sequence of one of SEQ ID NOs: 42-53;
(d) the EGFR binding specificity is represented by the amino acid sequence of one of SEQ ID NOs: 55-83;
(e) the HSA binding specificity is represented by the amino acid sequence of one of SEQ ID NOs: 114-118; and
(f) the IL-22 binding specificity is represented by the amino acid sequence of one of SEQ ID NOs: 121-124.

9. The multi-specific antibody of claim 8, further comprising one to five additional antibody binding domains, wherein each additional antibody binding domain is individually specific for OX40, CD40, 4-1BB, HSA, IL-22, or EGFR.

10. The multi-specific antibody of claim 9, further comprising one to four additional antibody binding domains, wherein each additional antibody binding domain is specific for OX40, CD40, 4-1BB, HSA, IL-22, or EGFR.

11. The multi-specific antibody of any one of claims 8-10, wherein the antibody is a multi-specific single chain antibody (MVSCA).

12. The multi-specific antibody of any one of claims 8-11, wherein the linker is selected from a linker of Table 26.

13. The multi-specific antibody of claim 12, wherein linker L1 (SEQ ID NO:84), L2 (SEQ ID NO:85), or L4 (SEQ ID NO:87) are interposed between one or more pairs of non-identical antibody-binding domains.

14. The multi-specific antibody of any one of claims 8-12, comprising at least one pair of antibody binding domains with the same specificity.

15. The multi-specific antibody of claim 14, wherein the at least one pair of antibody-binding domains with the same specificity are adjacent to each other.

16. The multi-specific antibody of claim 15, wherein a linker having the amino acid sequence of linker L3 (SEQ ID NO:86) is interposed between the antibody-binding domains of the same specificity.

17. The multi-specific antibody of any one of claims 8-16, wherein all of the antibody binding domains are VHH domains.

18. The multi-specific antibody of any one of claims 8-17, wherein the multi-specific antibody has the amino acid sequence of one of SEQ ID NOs: 107-112.

19. A pharmaceutical composition comprising the VHH domain of any one of claims 1-6, the antibody of claim 7, or the multi-specific antibody of any one of clams 8-18.

20. A method of treating cancer comprising administering the pharmaceutical composition of claim 19 to a patient in need thereof.

21. A method of treating an autoimmune disease comprising administering the pharmaceutical composition of claim 19 to a patient in need thereof.


SEQUENCE LISTING (142 sequences)

SEQ ID NO: 1 - protein, 188 aa, Homo sapiens
LHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVA

SEQ ID NO: 2 - protein, 125 aa, synthetic construct
EVQLVESGGGLIQVGGSLTLSCVSSGSPFSSNAMGWYRQAPGKQREWLATISSVGDYTEYAPSVKGRFTISRDDARNTLYLQMNSLRSEDTAVYYCKRCQTWGFGMCPNTPEDLWGQGTQVTVSS

SEQ ID NO: 3 - protein, 114 aa, synthetic construct
EVQLVESGGGLVQPGGSLRLSCAASGFAFGDIPMMWVRQAPGKGREWVSSISADSATQSYADSVQDRFTISRDNAKNTLYLEMHILKPEDTAVYYCATTDRPPPGQGTQVTVSS

SEQ ID NO: 4 - protein, 127 aa, synthetic construct
QVQLVESGGGLVQAGGSLRLSCAASGGTFSTYAMGWFRQAPGKEPQFVARITWSGYTLYTEPVQGRFAISRDNAKNTVYLQMNSLKPEDTAEYFCAADSSEFRDIPNRDSRANYRYWGQGTQVTVSS

SEQ ID NO: 5 - protein, 127 aa, synthetic construct
EVQLVESGGGLVQPGGSLKLSCAASGFRWNYYSIFWFREAPGKEREGISRISGIDGRAYYADSVKGRFTIFRDSSKSTVYLQMNSLKPEDTARYYCATRPPQYGSTCPITPRLYAYRGQGTQVTVSS

SEQ ID NO: 6 - protein, 123 aa, synthetic construct
EVQLVESGGRLVQAGDSLKLSCTASGRTFSDYVMGWFRQVPGEGRLMVAVITPAGDTTEYADAVKGRFSISKDSAKSTVNLQMNNLKVDDTAVYYCAAGPSWIGMQFGTAEYWGQGTQVTVSS

SEQ ID NO: 7 - protein, 125 aa, synthetic construct
QVQLVESGGGLVQAGGSLTLSCVSSGSPFSSNAMGWYRQAPGKQRKWLATVSSVGDYTEYAPSVKGRFTISRDDARNTLYLQMNNLRSEDTAVYYCKRCQTWGFGMCPNTPEDLWGQGTQVTVSS

SEQ ID NO: 8 - protein, 125 aa, synthetic construct
EVQLVESGGGLVQPGGSLRLSCAASGIPFGSTAMGWYRQAPGLERELVACISSVGDHTNYADSVKGRFTVSRDNSKNTVYLQMNSLKPEDTAVYYCARGQSWMFGMCPVTAQDSWGQGTQVTVSS

SEQ ID NO: 9 - protein, 120 aa, synthetic construct
EVQLVESGGGLVQAGESLRLSCSASGSIRLNAVGWFRQAPVNQRLMVATITHNNSTNYADSVQGRFTIARDDAKNTVTLQMNSLKPEDTAVYMCKVYQLPVPISSHDDYWGQGTQVTVSS

SEQ ID NO: 10 - protein, 125 aa, synthetic construct
QVQLVESGGGLVQPGGSLRLSCAASGSTLSFHAMGWFRQVPGKQRELVAAISTVGDYTNYASSVEGRFTISRDNAVKVVYLQMNSLKPDDTAVYYCQHCRSWLFGTCPYTPQDYWGQGTQVTVSS

SEQ ID NO: 11 - protein, 125 aa, synthetic construct
EVQLVESGGDLVQPGGSLTLSCVGRGSPFGTHAMGWYRQIGAKVRELVAAISSGGDYTNYADSVKGRFTISRGVTNDTLYLQMNRLKPEDTAVYYCVRCQFWAFGTCPNTPQDSWSQGTQVTVSS

SEQ ID NO: 12 - protein, 120 aa, synthetic construct
QVQLVESGGGLVQAGESLNLSCSASGSIRNNAVAWFRQGPGNQRLRVAAITNTGTTDYADSVQGRFSIARDNAENTVTLQMNSLKPEDTAIYYCKIYTLPVPISSHDDTWGQGTQVTVSS

SEQ ID NO: 13 - protein, 126 aa, synthetic construct
EVQLVESGGGLVQAGGSLRLSCVATGRTRSSTYIMAWFRQAPGKERDFVAAISFIGGSPVYTDSVKGRFTISRVIAENTVHLQMNDLTPEDTALYYCAATDSGPLGPSGPSKYEYWGQGTQVTVSS

SEQ ID NO: 14 - protein, 133 aa, synthetic construct
QVQLVESGGGLVQPGGSLLLSCAASGFRWDYYSIAWFREAPGKEREGISCISGTDDRTYYADSVKDRFTISRDDARKTVYLEMKNLKPEDTAVYYCATGQVVFRPSADDSDDLFTGDCEYTKWGQGTQVTVSS

SEQ ID NO: 15 - protein, 126 aa, synthetic construct; identical to SEQ ID NO: 13

SEQ ID NO: 16 - protein, 125 aa, synthetic construct
QVQLVESGGGLVQAGGSLRLSCVSSGSPFSSNAMGWYRQAPGKQREWLATISSVGDYTEYAPSVKGRFTISRDDARNTLTLRMNSLRSEDTAVYYCKRCQTWGFGMCPNTPEDLWGQGTQVTVSS

SEQ ID NO: 17 - protein, 118 aa, synthetic construct
EVQLVESGGGLVQPGGSVTLSCATSKDTFSRYHMGWFRQAAGKEREFVAAIANTGTTTDYADFVKGRFTISRDSAKNTVYLQMDSLKVEDTAMYYCVVGSQFMSYDYWGQGTQVTVSS

SEQ ID NO: 18 - protein, 127 aa, synthetic construct
QVQLVESGGGLVQPGGSLRLSCSPSEGAFKMLTTGWFRQVLGKEREFVSAISWGGGATYYADSVKGRFTVSRDNAKNTVYLQMNSLKPEDTAVYYCAVDTGNGVATNIRSQSRYDYWGQGTQVTVSS

SEQ ID NO: 19 - protein, 120 aa, synthetic construct
QVQLVESGGGLAQPGSSLRLSCTASGTVFSILSATWFRQAPGKAHEARELVAFISSDGVSMHADSVKGRFTISRDNAASTVYLQMDNLQPEDTAVYFCKLSNSMMSLEYWGRGTQVTVSS

SEQ ID NO: 20 - protein, 126 aa, synthetic construct
EVQLVESGGGLAQPGGSLRLSCAASGFTFSNYAMGWYRQAPGKEREFVAAIDWSTNALLYGDSVKGRFTISRDNAKNTVFLQMSGLKPEDTALYYCAADRAYHTVVLGPPEGMDYWGKGTQVTVSS

SEQ ID NO: 21 - protein, 120 aa, synthetic construct
EVQLVESGGGLAQPGSSLRLSCTASGTVFSILSATWFRQAPGKAHEARELVAFISSDGVSMHADSVKGRFTISRDNAKSTVYLQMDNLQPEDTAAYYCKLSNSMMGMEYWGRGTQVTVSS

SEQ ID NO: 22 - protein, 127 aa, synthetic construct
EVQLVESGGGLVQTGDSLRLSCAALGGTPSTYAIGWFRQAAGKELQFVARITWNGDTWYADALPGRVAISRDNAKKTVYLQMNNLKPEDTAEYICAADVSEFRDIPNRDSRANYRYWGQGTQVTVSS

SEQ ID NO: 23 - protein, 123 aa, synthetic construct
EVQLVESGGRLVQAGDSLKLSCTASGRTFSDYVMGWFRQVPGEGRLMVAVITPAGDTTEYADAVKGRFSISKDSAKRTVNLQMNNLKVDDTAVYYCAAGPSWIGMQFGTAEYWGQGTQVTVSS

SEQ ID NO: 24 - protein, 136 aa, synthetic construct
EVQLVESGGGSVQSGGSLTLSCVASGSTLSTVAMAWFRQPPGKELQFVARIATNGYTYYAGGYTYYGIGDASQGRFTISRDDAKNTVYLQMNNLELEDTAVYYCAGDISEFRDIPNRDSRANYYYWGQGTQVTVSS

SEQ ID NO: 25 - protein, 173 aa, Homo sapiens
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLR

SEQ ID NO: 26 - protein, 120 aa, synthetic construct
QVQLVESGGALAQPGGSLRLSCEASTNVRSVNAMAWYRQGPGKPRELVANITSDDATYYADSVKGRFTLSREGARNTVYLQMNSLKPEDTAVYQCNVHYTSWGGIRRDYWGRGTQVTVSS

SEQ ID NO: 27 - protein, 120 aa, synthetic construct
EVQLVESGGALAQPGGSLRLSCEASATIRSVNAMAWYRQGPGKPREMVANITSGDTTYYADSVKGRFTLSREGARNTVYLQMNSLKPEDTAVYQCNIHYTSTGGIRRDYWGRGTQVTVSS

SEQ ID NO: 28 - protein, 124 aa, synthetic construct
QVQLVESGGGLVQAGDSLRLSCTASGRAFNTYTMAWFRQAPGKERDFVAAISRDGTITHYADSVKGRFTTTRDNAKNTMYLQMSSLKYDDTAVYYCAARRVGAVPERESAYEHWGQGTQVTVSS

SEQ ID NO: 29 - protein, 120 aa, synthetic construct
QVQLVESGGALAQPGGSLRLSCEASENVRSVNAMAWYRQGPGKPRELVANITSGDATYYADSVKGRFTLSREGARNTVYLQMNSLKPEDTAVYQCNVHYTSWGGIRRDYWGRGTQVTVSS

SEQ ID NO: 30 - protein, 120 aa, synthetic construct
QVQLVESGGALAQPGGSLRLSCEASATVRSVNAMAWYRQGPGKPRELVANITSGDTTYYADSVKGRFTLSREGARNTVYLQMNSLKPEDTAVYQCNIHYTSTGGIRRDYWGRGTQVTVSS

SEQ ID NO: 31 - protein, 120 aa, synthetic construct
EVQLVESGGGLVQPGGSLKLSCAASGFAFSRYTMSWVRQAPGKGLEWVSGIDSGGGRTSYINSVKGRFTISRDNAKNTLYLQMNSLKPEDTAVYHCAQGGDSGSYYRTSWGQGTQVTVSS

SEQ ID NO: 32 - protein, 123 aa, synthetic construct
QVQLVESGGGLVQAGGSLRLSCAVSGFMVSFNAMGWYRQAPGKQRELVGGISSGGATNYADSVRRRFTIFRDSAKNTVYLQMNSLKPEDTAVYYCNGGGSLSSVYNPHQYTYWGQGTQVTVSS

SEQ ID NO: 33 - protein, 123 aa, synthetic construct
EVQLVESGGGLVQAGGSLRLSCAASGFTFDDYAIGWFRQAPGKEREGVSCLSKNVGNTYYADSVKGRFTISSDNAKNTVYLQMNSLKPEDTAVYYCAKGSGYGCYDFPRYAVWGQGTQVTVSS

SEQ ID NO: 34 - protein, 124 aa, synthetic construct
EVQLVESGGGSVQAGGSLRLSCAASLSTFSNYRMGWFRRAPGKERELVARIEPHGVVDYGDSVKGRFTISRDNAKNTVTVYLHMNSLKPEDTAVYYCNALRLISGRYDPLFSSWGQGTQVTVSS

SEQ ID NO: 35 - protein, 122 aa, synthetic construct
EVQLVESGGGLVQAGGSLRLSCVASERTFTPVRMGWFRQAPGKEREIVAAITWSDGSTYYADAVKGRFTISRDNALNAVYLQIDSLKPEDTAVYVCAASTSPYSTHLQFRYWGQGTQVTVSS

SEQ ID NO: 36 - protein, 121 aa, synthetic construct
QVQLVESGGGLVQPGGSMRLSCSASGFSISVYWMSWVRRAPGKGLEWVSNLGADGVTAQYSDSVKGRFTIARDTAKNTLYLQMNSLKPEDTAVYYCNAMTGMNPAEEEAYWGQGTQVTVSS

SEQ ID NO: 37 - protein, 127 aa, synthetic construct
QVQLVESGGGLVQPGESLTLSCVTSGFVFSNHWMYWDRQAPGKGLEWVADINLGGEMTYYSNSVKGRFTISRDNAKNTVSLRMDNLKVEDTAVYFCAANQQTPPYGLGSREYDYDYWGQGTQVTVSS

SEQ ID NO: 38 - protein, 124 aa, synthetic construct
QVQLVESGGGLVQPGGSLRLSCSASGRAFNTYTMAWFRQAPGKERDFVAAISRDGTITYYADSVKGRFTISRDNSKNTLYLQMSSLRAEDTAVYYCAARRVGAVPERESAYEHWGQGTQVTVSS

SEQ ID NO: 39 - protein, 124 aa, synthetic construct
QVQLVESGGGLVQPGGSLRLSCSASGRAFNTYTMAWVRQAPGKGLEYVSAISRDGTITYYADSVKGRFTISRDNSKNTLYLQMSSLRAEDTAVYYCAARRVGAVPERESAYEHWGQGTQVTVSS

SEQ ID NO: 40 - protein, 124 aa, synthetic construct
EVQLVESGGGLVQPGGSLRLSCAASLSTFSNYRMGWFRQAPGKERELVARIEPHGVVYYADSVKGRFTISRDNAKNTVTVYLQMNSLRAEDTAVYYCNALRLISGRYDPLFSSWGQGTQVTVSS

SEQ ID NO: 41 - protein, 160 aa, Homo sapiens
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGH

SEQ ID NO: 42 - protein, 116 aa, synthetic construct
QVQLVESGGGLVQAGGSLRLSCAASENIFSFNAMAWYRQAPGKQRELVALITGDGTTKYADSVKGRFTIARLNAKNTVNLEMNSLKPEDTAVYTCAALRNSDRTSWGQGTQVTVSS

SEQ ID NO: 43 - protein, 116 aa, synthetic construct
QVQLVESGGGLVQAGGSLRLSCAASGSIFSYNAMAWYRQAPGKQRELVALITSDRTTKYADSVKGRFTISRDDTKNTVDLEMNSLKPEDTAVYTCAALLNSARTSWGQGTQVTVSS

SEQ ID NO: 44 - protein, 116 aa, synthetic construct
EVQLVESGGGLVQAGGSLRLSCAASGNIFSYNGMAWYRQAPGNQRELVAVVAIDGTAKYADTVKGRFTISRDNIKNTVNLEMNSLKPEDTAVYTCAALRNSGSTSWGQGTQVTVSS

SEQ ID NO: 45 - protein, 117 aa, synthetic construct
QVQLVESGGGLVQAGGSLRLSCAASGSIFSYNAVAWYRQAPGKQRELVAVITSGGETTKYADSVKGRFTISRDITKNAVDLQMVSLKPEDTAVYTCAALRNSGSTSWGQGTQVTVSS

SEQ ID NO: 46 - protein, 116 aa, synthetic construct
EVQLVESGGGLVQAGGSLRLSCAASGAIFSYNGMAWYRQAPGKQREFVAVITPDGTTKYGDSVKGRFTISRDITKNTVDLEMNSLKPEDTAVYTCAALRNSGSTSWGQGTQVTVSS

SEQ ID NO: 47 - protein, 119 aa, synthetic construct
EVQLVESGGGLVQPGGSLRLSCAASRRTFSSYTAGWFRQAPGKEREFVASIRWISGTTTYADSVNGRFTISRDIAKNTVYLQMNSLKPDDTAVYYCAVDLSSDNDYDYWGQGTQVTVSS

SEQ ID NO: 48 - protein, 123 aa, synthetic construct
QVQLGSLGGGSAQPGGSLRLSCTGSGNVYDFVVMGWYRQFPGRQRELVANITGRASPNYVDSVKGRFTISRDNAQNTVHLQMNNLKVEDSAIYYCYAAQQLTTIAGVMTLDYWGSGTQVTVSS

SEQ ID NO: 49 - protein, 118 aa, synthetic construct
QVQLVESGGGLVQTGGSLRLSCAASGRISTIDTMFWYRQTPGKERDWVAFIPKDGLPTYVDSVKGRFTVSRDNARNTVYLQMNGLKPEDTAVYYCAVDVQEDRMRTYWGQGTQVTVSS

SEQ ID NO: 50 - protein, 119 aa, synthetic construct
EVQLVESGGGLVQPGESLRLSCAASGDTVSGYVMLWWRQAPGKERELVAAISSVGYTYYTDSVKGRFTISRDNAKNTVYLQMNSLKPEDTAVYYCAADPASISTRSAYWGQGTQVTVSS

SEQ ID NO: 51 - protein, 118 aa, synthetic construct
QVQLVESGGGLVQPGGSLRLSCAASGRISTIDTMFWYRQTPGKERDWVAFIPKDGLPYYADSVKGRFTVSRDNSKNTLYLQMNSLRAEDTAVYYCAVDVQEDRMRTYWGQGTQVTVSS

SEQ ID NO: 52 - protein, 118 aa, synthetic construct
QVQLVESGGGLVQPGGSLRLSCAASGRISTIDTMFWYRQAPGKGLEWVAFIPKDGLPYYADSVKGRFTISRDNSKNTLYLQMNSLRAEDTAVYYCAVDVQEDRMRTYWGQGTQVTVSS

SEQ ID NO: 53 - protein, 119 aa, synthetic construct
EVQLVESGGGLVQPGGSLRLSCAASGDTVSGYVMLWWRQAPGKERELVSAISSVGYTYYADSVKGRFTISRDNAKNTLYLQMNSLRAEDTAVYYCAADPASISTRSAYWGQGTQVTVSS

SEQ ID NO: 54 - protein, 645 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCKLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPS

SEQ ID NO: 55 - protein, 112 aa, synthetic construct
QVQLVESGGGLVQPGGSLRLSCAASGIIFEAEAMGWVRQAPGKQRESVAFIGSGGNTNVGWSVKGRFTISRDNAKNTLYLQMNSLRPEDTAVYYCNTHPLRSWGQGTQVTVS

SEQ ID NO: 56 - protein, 120 aa, synthetic construct
QVQLVESGGGLVQPGGSLRLSCTASHNIFSDNAMAWSRQAPGAQRELVARIATGGNTYYPDSVKGRFTISRDNAKNTVYLQMNNLTPDDTAVYYCYAERWTGLEYSRTYWGQGTQVTVSS

SEQ ID NO: 57 - protein, 113 aa, synthetic construct
QVQLVESGGGLVQPGGSLRLSCVASGIIFEAEAMGWYRQAPGKQRESVAFIGSGGNTNVGWSVKGRFTISRDNAKNTVYLQMNSLKPEDTAVYYCNTHPLRSWGQGTQVTVSS

SEQ ID NO: 58 - protein, 115 aa, synthetic construct
QVQLVESGGGLVQAGGSLSLSCTASRNIFSINVMSWYRQAPGKQREFVASITREDFPYYTDSVKGRFTVTRDIANNKVHLQMNSLKLEDTAVYYCNAYYRSDNYWGQGTQVTVSS

SEQ ID NO: 59 - protein, 113 aa, synthetic construct
QVQLVESGGGLVQAGGSLRLSCVVYGSIFASTTMGWYRQAPGKQRELVATVYQSGTSVYADSVKGRFTASRDNAKKTVYLQMNSLKPEDTAVYYCHPRATDYWGRGTQVTVSS

SEQ ID NO: 60 - protein, 121 aa, synthetic construct
QVQLVESGGGTVQAGGSLRLSCVASGMREDIFDMGWYRQAPGLQRELVATITSGGSTDYADSVKGRFTISRDNTENTLNLQMNMLKPEDTAVYYCNALYFPPSGGRSSEFWGQGTLVTVSS

SEQ ID NO: 61 - protein, 116 aa, synthetic construct
QVQLVESGGGLVQAGGSLRLSCAASVSGSALSNMAWYRQRPGNQRELVAHITFDNFTNYADSVKGRFTISRDNVKNTVDLQMSSLKPEDTAVYYCNARHFFGDNYWGKGTLVTVSS

SEQ ID NO: 62 - protein, 114 aa, synthetic construct
QVQLVESGGGLVQAGGSLRLSCAGARSAFSIKPMTWYRQAPGKERELVAYFNSGGSTNYADSVKGRFTISRDNAKNTMYLQMNNLKPEDTAVYYCNAIPPLGSWGQGTQVTVSS

SEQ ID NO: 63 - protein, 123 aa, synthetic construct
QVQLVESGGGLVQAGGSLRVSCAVSGGALSAYAMAWFRQAPGKEREFVAGLNWGGDETYYADSAKGRFTISKDNAKNTVSLQMNSLEPEDTAVYYCGGRPGPLLSRATGYKYWGQGTQVTVSS

SEQ ID NO: 64 - protein, 115 aa, synthetic construct
QVQVAESGGGLVQAGESLRLSCTASGSISTINAVRWYRQTPGKQREFVAYISTDGGTDYADPVKGRFTISRDNAKNTWFLQMNSLKPEDTGVYMCNVVVTPYAYWGQGTQVTVSS

SEQ ID NO: 65 - protein, 113 aa, synthetic construct
QVQLVESGGGLVQAGGSLRLSCTGSGIRFSFYTLGWYRQAPGKQRELVAEIYSSDNAKYEDSVKGRFAISRDNAKNSVSLQMNSLKPEDTAVYYCNVRATDYWGPGTQVTVSS

SEQ ID NO: 66 - protein, 117 aa, synthetic construct
QVQLVESGGGLVKAGKSLRLSCAASVSSFDTYTMGWYRQAPGKQREQVAIITPGAGTHYADSVKGRFTISRDNAKNTVYLQMSSLKLEDTAVYYCKARHRITGYDYWGQGTQVTVSS

SEQ ID NO: 67 - protein, 119 aa, synthetic construct
QVQLVESGGGLVQPGGSLRLSCAGTGLIFTTYVMGWYRQAPGKQRELVAIVTNGGGTHYADFVKGRFTISRDNAKNTVSLQMNLLKPEDTAVYYCNARHLVRPGTNDYWGQGTLVTVSS

SEQ ID NO: 68 - protein, 120 aa, synthetic construct
QVQLVESGGGFVQAGGSLRLSCAASGRFLSIADMDWYRRVPGKQRELVASITRAGDTALEDSVKGRFTISRDNAKNTVHLQMNSVKPEDTAVYYCRADVTRSGTPYFEVWGQGTLVTVSS

SEQ ID NO: 69 - protein, 114 aa, synthetic construct
QVQLVESGGGLVQAGGSLRLSCAGARSIFSIKPMTWYRQAPGKERELVAWFTSGDSPNYADSVKGRFTISRDSAKNTVYLQMNNLKPEDTAVYYCNAVPPLGRWGQGTLVTVSS

SEQ ID NO: 70 - protein, 122 aa, synthetic construct
QAQVQLVESGGGLVQPGGSLRLSCAASHNIFSDNAMAWVRQAPGAQRELVARIATGGNTYYADSVKGRFTISRDNAKNTLYLQMNNLRPEDTAVYYCYAERWTGLEYSRTYWGQGTQVTVSS

SEQ ID NO: 71 - protein, 120 aa, synthetic construct
QVQLVESGGGLVQPGGSLRLSCAASHNIFSDNAMAWVRQAPGAQRELVARIATGGNTYYADSVKGRFTISRDNAKNTLYLQMNNLRPEDTAVYYCYAERWTGLEYSRTYWGQGTQVTVSS

SEQ ID NO: 72 - protein, 113 aa, synthetic construct
QVQLVESGGGLVKPGGSLRLSCAASGIRFSFYTMNWVRQAPGKGLEWVSSIYSSDNAKYADSVKGRFTISRDNAKNSLYLQMNSLRAEDTAVYYCNVRATDYWGPGTQVTVSS

SEQ ID NO: 73 - protein, 113 aa, synthetic construct
QVQLVESGGGLVQPGGSLRLSCTASGIRFSFYTLGWVRQAPGKQRELVAEIYSSDNAKYADSVKGRFTISRDNAKNSVSLQMNSLRAEDTAVYYCNVRATDYWGPGTQVTVSS

SEQ ID NO: 74 - protein, 113 aa, synthetic construct
QVQLVESGGGLVQPGGSLRLSCTASGIRFSFYTLGWVRQAPGKQRELVAEIYSSDNAKYADSVKGRFTISRDNAKNSLYLQMNSLRAEDTAVYYCNVRATDYWGPGTQVTVSS

SEQ ID NO: 75 - protein, 113 aa, synthetic construct
QVQLVESGGGLVQPGGSLRLSCTASGIRFSFYTMNWVRQAPGKERELVAEIYSSDNAKYADSVKGRFTISRDNAKNSLYLQMNSLRAEDTAVYYCNVRATDYWGPGTQVTVSS

SEQ ID NO: 76 - protein, 113 aa, Synthetic construct
QVQLVESGGGLVQPGGSLRLSCTASGIRFSFYTLGWVRQAPGKQRELVSSIYSSDNAKYADSVKGRFTISRDNAKNSLYLQMNSLRAEDTAVYYCNVRATDYWGPGTQVTVSS

SEQ ID NO: 77 - protein, 114 aa, synthetic construct
QVQLVESGGGLVQPGGSLRLSCAASRSIFSIKPMSWIRQAPGKERELVAWFTSGDSPNYADSVKGRFTISRDNAKNTLYLQMNSLRAEDTAVYYCNAVPPLGRWGQGTLVTVSS

SEQ ID NO: 78 - protein, 114 aa, synthetic construct
QVQLVESGGGLVQPGGSLRLSCAGSRSIFSIKPMSWVRQAPGKERELVAWFTSGDSPNYADSVKGRFTISRDNAKNTLYLQMNSLRAEDTAVYYCNAVPPLGRWGQGTLVTVSS

SEQ ID NO: 79 - protein, 114 aa, synthetic construct
QVQLVESGGGLVQPGGSLRLSCAGARSIFSIKPMTWYRQAPGKERELVAWFTSGDSPNYADSVKGRFTISRDSAKNTLYLQMNSLRAEDTAVYYCNAVPPLGRWGQGTLVTVSS

SEQ ID NO: 80 - protein, 114 aa, synthetic construct
QVQLVESGGGLVQPGGSLRLSCAGARSIFSIKPMSWVRQAPGKERELVAWFTSGDSPNYADSVKGRFTISRDSAKNTLYLQMNSLRAEDTAVYYCNAVPPLGRWGQGTLVTVSS

SEQ ID NO: 81 - protein, 114 aa, synthetic construct
QVQLVESGGGLVQPGGSLRLSCAGARSIFSIKPMTWIRQAPGKERELVAWFTSGDSPNYADSVKGRFTISRDSAKNTLYLQMNSLRAEDTAVYYCNAVPPLGRWGQGTLVTVSS

SEQ ID NO: 82 - protein, 113 aa, synthetic construct
QVQLVESGGGLVQPGGSLRLSCTASGIRFSFYTLGWYRQAPGKGRELVAEIYSSDNAKYADSVKGRFTISRDNAKNSLYLQMNSLRAEDTAVYYCNVRATDYWGPGTQVTVSS

SEQ ID NO: 83 - protein, 113 aa, synthetic construct
QVQLVESGGGLVQPGGSLRLSCAASGIRFSFYTLGWYRQAPGKGRELVSEIYSSDNAKYADSVKGRFTISRDNAKNSLYLQMNSLRAEDTAVYYCNVRATDYWGPGTQVTVSS

SEQ ID NO: 84 - protein, 9 aa, synthetic construct
GGGGSGGGS

SEQ ID NO: 85 - protein, 9 aa, synthetic construct
  REPEAT 1..5: note = REPEAT = 1-35
  REPEAT 6..9: note = REPEAT = 0-35
GGGGSGGGS

SEQ ID NO: 86 - skipped

SEQ ID NO: 87 - protein, 5 aa, synthetic construct
  REPEAT 1..5: note = REPEAT = 1-35
GGGGS

SEQ ID NO: 88 - protein, 18 aa, synthetic construct
GGRGPLGLAGSRSAFGGS

SEQ ID NO: 89 - protein, 9 aa, synthetic construct
GSPLGLAGS

SEQ ID NO: 90 - protein, 16 aa, synthetic construct
GGSGPLGLAGSRSAFG

SEQ ID NO: 91 - protein, 18 aa, synthetic construct
GPLGLAGSRSAGGSQVQL

SEQ ID NO: 92 - protein, 15 aa, synthetic construct
GSGPLGLAARSAGGS

SEQ ID NO: 93 - protein, 16 aa, synthetic construct
GSGPLGLAARSAFGGS

SEQ ID NO: 94 - protein, 21 aa, synthetic construct
GGSGRSAPLGLARQARQVGGS

SEQ ID NO: 95 - protein, 19 aa, synthetic construct
GGSGRSAPLGLGRQARGGS

SEQ ID NO: 96 - protein, 21 aa, synthetic construct
GGSPLGLARQARGSGRSAGGS

SEQ ID NO: 97 - protein, 21 aa, synthetic construct
GGSGRSAPLGLARQARVVGGS

SEQ ID NO: 98 - protein, 10 aa, synthetic construct
GSRQARVVGS

SEQ ID NO: 99 - protein, 10 aa, synthetic construct
GSRQRRVVGS

SEQ ID NO: 100 - protein, 8 aa, synthetic construct
GSRQARGS

SEQ ID NO: 101 - protein, 8 aa, synthetic construct
GSRQRRGS

SEQ ID NO: 102 - protein, 9 aa, synthetic construct
GSRQARGGS

SEQ ID NO: 103 - protein, 9 aa, synthetic construct
GSRQRRGGS

SEQ ID NO: 104 - protein, 6 aa, synthetic construct
EPKSCD

SEQ ID NO: 105 - protein, 6 aa, synthetic construct
ASTKGP

SEQ ID NO: 106 - protein, 120 aa, synthetic construct
QVQLVESGGGLVQAGESLRLSCSASGNIRLNAVGWFRQGPGNQRLMVATITHNNSTNYADSVQGRFTIARDDAKNTVTLQMNSLKPEDTAVYYCKVYELPVPISSHDDDWGQGTQVTVSS

SEQ ID NO: 107 - protein, 494 aa, synthetic construct
EVQLVESGGGLVQPGGSLRLSCAASGGGRTFSNYALGWFRQAPGKEREFVAAISRSGGNINYADSVKGRFTISRDNFKNTLYLQMSSLRPEDTAVYYCAAHYLLLPSYISTSTNMYNYWGQGTQVTVKPGGGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYSSDIAVEWESNGQPENNYKTTPPVLDSDGSFFLYSKLTVDKSRWQQGNVFSCSVMHEALHNHYTQKSLSLSPGSGGGGSGGGGSQVQLVESGGGLVQPGGSLRLSCSASGRAFNTYTMAWFRQAPGKERDFVAAISRDGTITYYADSVKGRFTISRDNSKNTLYLQMSSLRAEDTAVYYCAARRVGAVPERESAYEHWGQGTQVTVSS

SEQ ID NO: 108 - protein, 494 aa, synthetic construct
QVQLVESGGGLVQPGGSLRLSCSASGRAFNTYTMAWFRQAPGKERDFVAAISRDGTITYYADSVKGRFTISRDNSKNTLYLQMSSLRAEDTAVYYCAARRVGAVPERESAYEHWGQGTQVTVKPGGGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYSSDIAVEWESNGQPENNYKTTPPVLDSDGSFFLYSKLTVDKSRWQQGNVFSCSVMHEALHNHYTQKSLSLSPGSGGGGSGGGGSEVQLVESGGGLVQPGGSLRLSCAASGGGRTFSNYALGWFRQAPGKEREFVAAISRSGGNINYADSVKGRFTISRDNFKNTLYLQMSSLRPEDTAVYYCAAHYLLLPSYISTSTNMYNYWGQGTQVTVSS

SEQ ID NO: 109 - protein, 494 aa, synthetic construct; identical to SEQ ID NO: 107

SEQ ID NO: 110 - protein, 509 aa, synthetic construct
QVQLVESGGGLVQPGGSLRLSCTASGIRFSFYTLGWYRQAPGKGRELVAEIYSSDNAKYADSVKGRFTISRDNAKNSLYLQMNSLRAEDTAVYYCNVRATDYWGPGTQVTVSSGGGGSGGGSEVQLVESGGGLVQPGGSLRLSCAASGSTSGIYDMGWYRQAPGKLREVVSVITSGGTTYYADSVKGRFTISRDNSKNTLYLQMNSLRAEDTAVYYCNIRTRLIIWGQGTQVTVSSGGGGSGGGSQVQLVESGGGLVQPGGSLRLSCSASGRAFNTYTMAWFRQAPGKERDFVAAISRDGTITYYADSVKGRFTISRDNSKNTLYLQMSSLRAEDTAVYYCAARRVGAVPERESAYEHWGQGTQVTVSSGGGGSGGGGSGGGGSEVQLVESGGGLVQPGGSLRLSCAASGRTDSAYRMGWFRQAPGKEREFVSAINWSDGRTVYLDSVKGRFTISRDNAKNTLYLQMNSLRAEDTAVYYCAADPDSRLYYTVPQNYDYWGQGTQVTVSS

SEQ ID NO: 111 - protein, 503 aa, synthetic construct
QVQLVESGGGLVQPGGSLRLSCTASGIRFSFYTLGWYRQAPGKGRELVAEIYSSDNAKYADSVKGRFTISRDNAKNSLYLQMNSLRAEDTAVYYCNVRATDYWGPGTQVTVSSGGGGSGGGSEVQLVESGGGLVQPGGSLRLSCAASGSTSGIYDMGWYRQAPGKLREVVSVITSGGTTYYADSVKGRFTISRDNSKNTLYLQMNSLRAEDTAVYYCNIRTRLIIWGQGTQVTVSSGGGGSGGGSEVQLVESGGGLVQPGGSLRLSCAASGRTDSAYRMGWFRQAPGKEREFVSAINWSDGRTVYLDSVKGRFTISRDNAKNTLYLQMNSLRAEDTAVYYCAADPDSRLYYTVPQNYDYWGQGTQVTVSSGGGGSGGGSQVQLVESGGGLVQPGGSLRLSCSASGRAFNTYTMAWFRQAPGKERDFVAAISRDGTITYYADSVKGRFTISRDNSKNTLYLQMSSLRAEDTAVYYCAARRVGAVPERESAYEHWGQGTQVTVSS

SEQ ID NO: 112 - protein, 476 aa, synthetic construct
QVQLVESGGGLVQPGGSLRLSCTASGIRFSFYTLGWYRQAPGKGRELVAEIYSSDNAKYADSVKGRFTISRDNAKNSLYLQMNSLRAEDTAVYYCNVRATDYWGPGTQVTVKPGGGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYSSDIAVEWESNGQPENNYKTTPPVLDSDGSFFLYSKLTVDKSRWQQGNVFSCSVMHEALHNHYTQKSLSLSPGGGGSGGGGSGGGGSQVQLVESGGGLVQPGGSLRLSCRASGFTFSNHAMSWVRQAPGKGLEWVSEISFNGHATRYADSVKGRFTISRDNSKNTLYLQMNSLRAEDTAVYYCRKGWNATPQIGERGRGTQVTVSS

SEQ ID NO: 113 - protein, 609 aa, Homo sapiens [sequence omitted]

SEQ ID NO: 114 - protein, 123 aa, synthetic construct
EVQLVESGGGLAQAGGSLRLSCADSGHSFSLYAMAWFRQTPEKEREFVAAISSSGGDTYYADSVKGRFTISRENAKNTAYLQMDSLKPGDTAVYFCAGNYYTSQLMRGMYEHWGQGTQVTVSS

SEQ ID NO: 115 - protein, 123 aa, synthetic construct
EVQLVESGGGLAQAGGFLRLSCADSGHSFSLYAMAWFRQTPEKEREFVAAISSSGGDTYYADSVKGRFTISRENAKNTVYLQMDSLKPGDTAVYFCAGNYYTSQLMRGMYEHWGQGTQVTVSS

SEQ ID NO: 116 - protein, 125 aa, synthetic construct
EVQLVEFGGGLVQAGGSLRLSCAASGRTDSAYRMGWFRQAPGKEREFVSAINWSDGRTVYLDSVKGRFTISRDNAKNMVYLQMNSLKPEDTAVYYCAADPDSRLYYTVPQNYDYWGQGTQVTVSS

SEQ ID NO: 117 - protein, 116 aa, synthetic construct
QVQLVESGGGLVQPGGSLRLSCAASGLPSRVMGWFRQAAGKEREFVASISVSGIDTRYGDSVKGRFTISRDNTKSSLYLQMNSLKPEDTAVYYCAATDGQGNYRHWGQGTQVTVSS

SEQ ID NO: 118 - protein, 128 aa, synthetic construct
EVQLVESGGGLVQPGGSLHLSCAASRLTTGVTFSDYGMAWFRQAPGAERELLANINWSGQYTNYRDSVKGRAAIFRDNAKNTLYLNINSLKAEDTAVYYCAASSRKYGPGIKSQYEWWGQGTQVTVSS

SEQ ID NO: 119 - protein, 125 aa, synthetic construct
EVQLVESGGGLVQPGGSLRLSCAASGRTDSAYRMGWFRQAPGKEREFVSAINWSDGRTVYLDSVKGRFTISRDNAKNTLYLQMNSLRAEDTAVYYCAADPDSRLYYTVPQNYDYWGQGTQVTVSS

SEQ ID NO: 120 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 121 - protein, 118 aa, synthetic construct
QVQLVESGGGLVQPGGSLRLSCEASGRTFSSVSMGWFRQAPGKERVIVAAADWSGTTYYTGSLKGRFTISRDNAKNMVYLQMNSLKPEDTAVYYCAASDPRRSAYKYWGQGTQVTVSS

SEQ ID NO: 122 - protein, 126 aa, synthetic construct
QVQLVESGGGLAQPGGSLRLSCAVSGFTLNYYVIAWFRQAPGKEREGVSCISSSDGSTYYTDSVKGRFTISRDNAKNTVYLQMDSLKPEDTAVYYCAADLRAYYTVPLWASEYDYWGQGTQVTVSS

SEQ ID NO: 123 - protein, 126 aa, synthetic construct
EVQLVESGGGLVQPGGSLRLSCAASGFTLDYYAIAWFRQAPGKEREGVSCISTSDGSTYYADSVKGRFTISRDNAKNTVYLQMNSLKPEDTAVYYCAADPSPFFVAPLVDYEYDYWGQGTQVTVSS

SEQ ID NO: 124 - protein, 123 aa, synthetic construct
EVQLVESGGGLVQPGGSLRLSCAASGFTLNYYVMSWFRQAPGKEREGVSAISSSDGSTYYADSVKGRFTISRDNSKNTLYLQMNSLRAEDTAVYYCAADLRAYYTVPLWASEYDYWGQGTVSS

SEQ ID NO: 125 - protein, 124 aa, synthetic construct
QVQLVESGGGLMQPGGSLRLSCAASGGPLSSYAMAWFRQAPGKEREFVTFISYNGGRIFYADSVKGRFTISRDNAKNTVSLQMNSLKPEDTAVYYCAGGLRSSNYVKPSQYNYWGRGTQVTVSS

SEQ ID NO: 126 - protein, 119 aa, synthetic construct
EVQLVESGGSLVQPGGSLRLSCAASGFNFNIYWMDWVRQAPGKGLEWISGISSSGGTTNYADSVKGRFTISRDNAKNTLYLQMNSLKSEDTAVYYCARSSGLWYGMDSWGKGTQVTVSS

SEQ ID NO: 127 - protein, 120 aa, synthetic construct
EVQLVESGGGLVQPGGSLRLSCVASGFTFSNYWMNWVRQAPGKGLEWVSTINTGGGSTTYTDSVKGRFTISRDNAKNTVYLQMNSLKPEDTALYYCNAPRWGTSREDDFWGQGTQVTVSS

SEQ ID NO: 128 - protein, 114 aa, synthetic construct
QVQLVESGGGLVQTGGSLRLSCEASGFDFSNSPMIWVRQAPGKGREWVSAISGDSRTQSYSDSVQDRFTISRDNTKSTLYLHMHSLKPEDTAVYYCSKTDDDPPGQGTQVTVSS

SEQ ID NO: 129 - protein, 118 aa, synthetic construct
EVQLVESGGGLVQPGGSVTLSCATSRDTFSRYHMGWFRQAVGKEREFVAAIARTGTTRDYADFVKGRFTISRDSAKNTVYLQMDSLKVEDTAMYYCVVGSQFMSYDYWGQGTQVTVSS

SEQ ID NO: 130 - protein, 125 aa, synthetic construct
EVQLVESGGGLVQPGGSLRLSCAASGIPFGSTAMGWYRQAPGLERELVACISSVGDHTNYASSVKGRFTISRDNSKDTVYLQMNSLKPEDTAVYYCARGQGWVFGMCPVAAQDSWGQGTQVTVSS

SEQ ID NO: 131 - protein, 120 aa, synthetic construct
QVQLVESGGGLVQAGESLRLSCSASGSIRLNAVGWWRQGPGDQRLMVATITHNGDTNYADSVQGRFTIARDDDKNTVTLQMNSLRPDDTAVYWCKVYQLQVPISSHDDSWGQGTQVTVSS

SEQ ID NO: 132 - protein, 126 aa, synthetic construct
QVQLVESGGGSVQAGGSLRLSCVASGSTAGVNVMGWYRQAPGNQRDMVASFSFGGIPNYAEPVKGRFTISRDHSKKNLDLQMNSLLPEDTGVYVCRARRPSGMFGGTWYDSPTDYWGQGIQVTVSS

SEQ ID NO: 133 - protein, 125 aa, synthetic construct
QVQLVESGGGLVQPGGSLRLSCAASGIPFGRTAMGWYRQAPGLERELVACISSVGDHTNYADSVKGRFTISRDNSKNTVSLQMNNLKPEDTAVYYCARGQSWMFGMCPVTAQDSWGQGTQVTVSS

SEQ ID NO: 134 - protein, 126 aa, synthetic construct
QVQLVESGGGSVQAGGSLRLSCEASGSSFVYSYMYWYRQAPGKERDLVASINSGGRTNYGDSVKGRFTISRDGAENIMYLQMNSLTPEDTAVYYCKVRRPSGQFGGVWYSDADEFWGQGTQVTVSS

SEQ ID NO: 135 - protein, 114 aa, synthetic construct
QVQLVESGGGLVQPGGSLRLSCSASGFAFSNLPMMWVREAPGKGREWVASISADSRTQSYADSVENRFWISRDNAKNALYLQMNRLRPEDTAVYYCTRTDGDPPGQGTQVTVSS

SEQ ID NO: 136 - protein, 124 aa, synthetic construct
QVQLVESGGGLVQVGGSLTVSCVSSGSPFSSNAMGWYRQAPGKQREWVATISSVGDYTEYAPSVKGRFTISRDDARNTLYLQMNSLRSEDTAVYYCKRCQTWGFGMCNTPEDLWGQGTQVTVSS

SEQ ID NO: 137 - protein, 127 aa, synthetic construct
PGQVQLVESGGGLVQPGGSLRLSCAASGIPFGSIAMGWYRQAPGLERALVACISSAGDHTNYASSVKGRFTISRDNSKDTVYLQMNSLKPEDTAVYYCARGQGWVFGMCPVTAQDSWGQGTQVTVSS

SEQ ID NO: 138 - protein, 125 aa, synthetic construct
QVQLVESGGGLVQAGGSLRLSCATSGVPFSSNAMGWYRQAPGKERELVAVISSGGDYTNYADSVKGRFTISRVMNNNTLYLQMNSLKPEDTAVYYCARCQSWRFGTCPVTAQDSWGQGTQVTVSS

SEQ ID NO: 139 - protein, 125 aa, synthetic construct
QVQLVESGGGLVQPGGSLRLSCAASGSTLSFHAMGWFRQAPGKERELVAAISTVGDYTNYASSVEGRFTISRDNAMKVVYLQMDSLKPDDTAVYYCQHCRSWLFGSCPYTPQDYWGQGTQVTVSS

SEQ ID NO: 140 - protein, 125 aa, synthetic construct; identical to SEQ ID NO: 10

SEQ ID NO: 141 - protein, 120 aa, synthetic construct
QVQLVESGGGVVEPGQSLRLSCSASENIRLNAVGWWRQGPGNQRLMVATITHNNSTNYGDSVQGRFTIARDDAKSTVTLQMNSLKPEDTAVYYCKIYQLPVPISSHDDYWGQGTQVTVSS

SEQ ID NO: 142 - protein, 126 aa, synthetic construct
QVQLVESGGGSVQAGGSLRLSCVASGSTAGVNVMGWYRQAPGNQRDMVASFSFGGIPNYAEPVKGRFAISRDNSRKNLDLQMNSLLPEDTGVYVCRARRPSGMFGGTWYDSPTDYWGQGIQVTVSS